(19) United States
(12) Patent Application Publication
(10) Pub. No.: US 2026/0055442 A1
(43) Pub. Date: Feb. 26, 2026

(54) IMAGE BASED AUTOFOCUS OF OPTICAL SYSTEMS

(71) Applicant: Element Biosciences, Inc., San Diego, CA (US)

(72) Inventors: Yanfei JIANG, San Diego, CA (US); Steve Xiangling CHEN, San Diego, CA (US); Jordan NEYSMITH, San Diego, CA (US); Michael PREVITE, San Diego, CA (US)

(21) Appl. No.: 19/285,769

(22) Filed: Jul. 30, 2025

Related U.S. Application Data
(63) Continuation of application No. PCT/US2024/015602, filed on Feb. 13, 2024.
(60) Provisional application No. 63/484,723, filed on Feb. 13, 2023.

(51) Int. Cl. C12Q 1/6825; C12Q 1/6844; H04N 23/56; H04N 23/67

(52) U.S. Cl. CPC C12Q 1/6825; C12Q 1/6844; H04N 23/56; H04N 23/67

(57) ABSTRACT

The present disclosure describes illumination methods and systems for illumination as well as methods and systems for autofocusing the systems. The systems can be used for, for example, microscopy and sequencing platforms. The methods and systems of the present disclosure can provide fast and accurate autofocusing, which can reduce error and improve system throughput.

### CROSS-REFERENCE

This application is a continuation of International Application No. PCT/US2024/015602, filed Feb. 13, 2024, which claims priority to, and the benefit of, U.S. Provisional Application No. 63/484,723, filed Feb. 13, 2023, the entire contents of each of which are incorporated herein by reference.

### BACKGROUND

In many fluorescence-based genomic testing assays, e.g., genotyping or nucleic acid sequencing, dye molecules that are attached to nucleic acid molecules tethered on a substrate are excited using an excitation light source, a fluorescent signal is generated in spatially-localized position(s) on the substrate, and the fluorescence is subsequently imaged through an optical system onto an image sensor. An analysis process is then used to analyze the images, find the positions of labeled molecules (or clonally amplified clusters of molecules) on the substrate, and quantify the fluorescence photon signal in terms of wavelength and spatial coordinates. This process may then be correlated with the degree to which a specific chemical reaction, e.g., a hybridization event or base addition event, occurred in the specified locations on the substrate. Imaging-based methods provide large scale parallelism and multiplexing capabilities, which help to drive down the cost and accessibility of such technologies.

### SUMMARY

Described herein are methods and systems for autofocusing optical systems, e.g., optical systems for imaging sequencing reactions, so that optical signals can be acquired in-focus and relied upon for generating accurate sequencing analysis results. The systems and methods described herein can utilize a single image to conveniently and accurately determine a z shift for autofocusing the optical system. The single image can be acquired using an image sensor of the optical system after tilting the sample stage relative to the image sensor, without the need for any dedicated hardware, e.g., an autofocus (AF) laser or an AF sensor, which are used for autofocusing purposes only. The image-based autofocusing methods and systems described herein advantageously save machinery costs and reduce complexity of the optical system compared to existing autofocusing methods using AF lasers and/or AF sensors. Additionally, the methods and systems herein require only a single image, which reduces time consumption and computational complexity when compared with existing autofocusing methods that rely on at least two images and/or machine learning algorithms.

The present disclosure provides for a method for focusing an optical system, comprising: receiving an image of a substrate of the optical system, wherein a portion and less than all of the image is in focus, and wherein the portion of the image in focus is offset from a center of the image; determining, using at least a distance from the portion of the image in focus and the center of the image, an amount of defocus in the image; and adjusting a parameter of the optical system to adjust for the defocus. In some embodiments, the image is an image of a flow cell, and wherein the substrate is a flow cell. In some embodiments, the adjusting of (c) is an automated adjusting. In some embodiments, the image is received from an autofocus element. In some embodiments, the determining is done in at most about 600 milliseconds (ms). In some embodiments, the determining is done within at most about 100 ms. In some embodiments, the method further comprises, prior to (a), imaging a substrate using a light source and a detector to generate the image. In some embodiments, the determining is performed using the image and no additional images. In some embodiments, the image comprises a length or width that is in a range from about 0.1 millimeters (mm) to about 5 centimeters (cm). In some embodiments, the image comprises a length or width that is in a range from about 0.5 mm to about 9 mm. In some embodiments, an error in the amount of defocus from a true amount of defocus is at most about 400 nanometers (nm). In some embodiments, an error in the amount of defocus from a true amount of defocus is at most about 100 nanometers (nm). In some embodiments, an error in the amount of defocus from a true amount of defocus is at most about 50 nanometers (nm). In some embodiments, a center of the in focus region is determined using an image processing algorithm. In some embodiments, the image processing algorithm comprises determining the center of the in focus region by separating the image into a predetermined number of regions and using a sum or average intensity of each region to identify the location of the in focus region. In some embodiments, image intensity or spatial frequency information of the location of the in focus region is used to locate the center of the in focus region. In some embodiments, information about a geometrical pattern in the image determines the image processing algorithm.

The present disclosure provides for a method of focusing an optical system, comprising: imaging, using a detector, a substrate tilted at a tilt angle, wherein an image of the substrate comprises an in focus portion and an out-of-focus portion; determining, using a processor, a defocus of the optical system based at least in part on the tilt angle and a distance of the in focus portion from a center of the image; adjusting the substrate to remove the tilt angle; and adjusting the substrate by the defocus, thereby focusing the optical system. In some embodiments, the determining of (b) further comprising using a vector of the in focus portion from the center of the image. In some embodiments, the method further comprises a motor coupled to the substrate configured to impart the tilt angle. In some embodiments, the detector is a portion of an autofocusing element. In some embodiments, the optical system further comprises an additional detector configured to image the substrate. In some embodiments, the method further comprises, prior to (a), tilting the substate to the tilt angle. In some embodiments, the method further comprises, subsequent to (d), de-tilting the substrate. In some embodiments, the tilting is tilting of a plane orthogonal to an optical axis of the optical system. In some embodiments, the tilt angle is from about 0.01 to about 89 degrees. In some embodiments, the tilt angle is from about 0.05 to about 15 degrees. In some embodiments, an angular resolution of the tilt angle is from about 0.001 degrees to about 0.2 degrees. In some embodiments, an angular resolution of the tilt angle is from about 0.01 degrees to about 0.1 degrees. In some embodiments, an angular resolution of the tilt angle is from about 0.01 degrees to about 0.08 degrees. In some embodiments, the determining is performed using the image and no additional images. In some embodiments, the substrate comprises a flow cell comprising: one or more surfaces; at least one hydrophilic polymer coating layer; a plurality of oligonucleotide molecules attached to the at least one hydrophilic polymer coating layer; and at least one discrete region of the one or more surfaces that comprises a plurality of clonally-amplified nucleic acid molecules immobilized to the plurality of attached oligonucleotide molecules, wherein the plurality of immobilized clonally amplified sample nucleic acid molecules are present at a distance less than λ/(2*NA), wherein λ is the center wavelength of an excitation energy source and NA is the numerical aperture of the optical system. In some embodiments, the substrate comprises a beaded flow cell. In some embodiments, the beaded flow cell comprises a surface comprising fluorescent beads chemically immobilized to the substrate. In some embodiments, the fluorescent beads are randomly distributed on the surface. In some embodiments, the fluorescent beads comprise at least about 4 types of beads configured to emit different colors in response to excitation from a laser. In some embodiments, an error in the distance from a focal plane to a true distance from the focal plane is at most about 400 nanometers (nm). In some embodiments, an error in the distance from the focal plane to a true distance from the focal plane is at most about 100 nanometers (nm). In some embodiments, an error in the distance from the focal plane to a true distance from the focal plane is at most about 50 nanometers (nm). In some embodiments, (d) occurs prior to the optical system imaging a nucleic acid molecule immobilized to the substrate in a first flow cycle. In some embodiments, the method further comprises repeating (a)-(d) to refocus the optical system for a second flow cycle.

The present disclosure provides a method of focusing an optical system, comprising: imaging, using a detector tilted at a tilt angle, a substrate, wherein an image of the substrate comprises an in focus portion and an out-of-focus portion; determining, using a processor, a defocus of the optical system based at least in part on the tilt angle and a distance of the in focus portion from a center of the image; and adjusting the substrate by the defocus, thereby focusing the optical system. In some embodiments, the method further comprises adjusting the substrate by the defocus, thereby placing the substrate into focus. In some embodiments, the method further comprises, prior to (a), tilting the detector to the tilt angle. In some embodiments, the method further comprises, subsequent to (c), de-tilting the detector. In some embodiments, the tilting is tilting of a plane orthogonal to an optical axis of the optical system. In some embodiments, the tilt angle is from about 0.01 to about 89 degrees. In some embodiments, the tilt angle is from about 0.05 to about 15 degrees. In some embodiments, the determining is performed using the image and no additional images. In some embodiments, an error in the amount of defocus from a true amount of defocus is at most about 400 nanometers (nm). In some embodiments, an error in the amount of defocus from a true amount of defocus is at most about 100 nanometers (nm). In some embodiments, an error in the amount of defocus from a true amount of defocus is at most about 50 nanometers (nm). In some embodiments, the method further comprises calibrating a pivot point of the optical system. In some embodiments, the calibrating of the pivot point comprises de-tilting the substrate, the detector, or an autofocus sensor.

The present disclosure provides for a method for autofocus of an optical system, comprising: tilting a sample stage of the optical system by a tilt angle, wherein a sample is immobilized on the sample stage; obtaining, by an image sensor of the optical system, an image of the sample on the tilted sample stage; determining, by a processor, a z shift based on: the tilt angle; and a x-y plane shift from a center of the image, wherein the x-y plane shift is determined based on an in-focus region of the image; and moving the sample stage relative to a focal plane of an objective lens of the optical system by the determined z shift thereby bringing the sample in-focus.

The present disclosure provides for a method for autofocus of an optical system, comprising: tilting an image sensor of the optical system by a tilt angle; obtaining, by the tilted image sensor of the optical system, an image of a sample, wherein the sample is immobilized on a sample stage; determining, by a processor, a z shift based on: the tilt angle; and a x-y plane shift from a center of the image, wherein the x-y plane shift is determined based on an in-focus region of the image; and moving the sample stage relative to a focal plane of an objective lens of the optical system by the determined z shift thereby bringing the sample in-focus.

The present disclosure provides for a method for autofocus of an optical system, comprising: tilting a sample stage of the optical system by a tilt angle, wherein a sample is immobilized on the sample stage; obtaining, by an autofocus (AF) sensor of the optical system, an image of the sample on the tilted sample stage, wherein the AF sensor is different from an image sensor of the optical system; determining, by a processor, a z shift based on the tilt angle and a x-y plane shift from a center of the image, wherein the x-y plane shift is determined based on an in-focus region of the image; and moving the sample stage relative to a focal plane of an objective lens of the optical system by the determined z shift thereby bringing the sample in-focus.

The present disclosure provides for a method for autofocus of an optical system, comprising: tilting an AF sensor of the optical system by a tilt angle, wherein the AF sensor is different from an image sensor of the optical system; obtaining, by the AF sensor of the optical system, an image of the sample, wherein the sample is immobilized on a sample stage; determining, by a processor, a z shift based on the tilt angle and a x-y plane shift from a center of the image, wherein the x-y plane shift is determined based on an in-focus region of the image; and moving the sample stage relative to a focal plane of an objective lens of the optical system by the determined z shift thereby bring the sample in-focus with the optical system. In some embodiments, the method further comprises: calibrating a pivot point of the optical system. In some embodiments, calibrating the pivot point of the optical system comprises: tilting the sample stage, the image sensor, or the AF sensor by the tilt angle or a second tilt angle; acquiring, by the AF sensor or the image sensor, a calibration image of the sample immobilized on the sample stage; determining, by the processor, a pivot point offset based on a region center of an in-focus region of the calibration image and an image center of the calibration image; and de-tilting the sample stage, the image sensor, or the AF sensor by the tilt angle or the second tilt angle. In some embodiments, the method further comprises: de-tilting the tilted sample stage by the tilt angle. In some embodiments, the method further comprises: de-tilting the tilted image sensor by the tilt angle. In some embodiments, the method further comprises: de-tilting the tilted AF sensor by the tilt angle. In some embodiments, tilting the sample stage of the optical system by the tilt angle is about a x or y axis. In some embodiments, tilting the sample stage of the optical system by the tilt angle is within a x-z plane or y-z plane. In some embodiments, tilting the AF sensor or image sensor of the optical system by the tilt angle is about x or y axis. In some embodiments, tilting the AF sensor or image sensor of the optical system by the tilt angle is within a x-z plane or y-z plane. In some embodiments, the tilt angle is in a range from 0.01 degrees to 89 degrees. In some embodiments, the tilt angle is in a range from 0.05 degrees to 15 degrees. In some embodiments, the tilt angle is clockwise about the x or y axis. In some embodiments, the tilt angle is counter-clockwise about the x or y axis. In some embodiments, the image of the sample obtained by the AF sensor or image sensor comprises a single image. In some embodiments, the AF sensor is only used for acquiring signals for autofocusing the optical system. In some embodiments, the image sensor is used for autofocusing the optical system and for imaging using the optical system after autofocusing. In some embodiments, the optical system lacks an AF illumination source that is only used for autofocusing but not for imaging. In some embodiments, the method for autofocus of the optical system is completed in 100 to 990 milliseconds. In some embodiments, the method for autofocus of the optical system is completed in less than 600 milliseconds. In some embodiments, the image comprises a field of view (FOV) that is identical to a size of the image sensor or the AF sensor along the x or y axis. In some embodiments, the image comprises a length or width that is in a range from 0.1 mm to 5 cm. In some embodiments, the image comprises a length or width that is in a range from 0.5 mm to 9 mm. In some embodiments, the image comprises a field of view (FOV) that is identical to a size of the image sensor or the AF sensor along the x axis when the tilt angle is about the x axis and along the y axis when the tilt angle is about the y axis. In some embodiments, the AF illumination source comprises a laser. In some embodiments, the image comprises fluorescent signal from the sample. In some embodiments, the sample comprises a flow cell comprising: one or more surfaces and one or more substrates; at least one hydrophilic polymer coating layer; a plurality of oligonucleotide molecules attached to at least one hydrophilic polymer coating layer; and at least one discrete region of the one or more surfaces that comprises a plurality of clonally-amplified, sample nucleic acid molecules immobilized to the plurality of attached oligonucleotide molecules, wherein the plurality of immobilized clonally amplified sample nucleic acid molecules are present at a distance less than λ/(2*NA), wherein λ is the center wavelength of an excitation energy source and NA is the numerical aperture of the optical system. In some embodiments, the sample comprises a beaded flow cell. In some embodiments, the beaded flow cell comprises a surface coated with fluorescent beads that are chemically immobilized to the surface. In some embodiments, the fluorescent beads are randomly distributed on the surface. In some embodiments, the fluorescent beads comprise one, two, three, four, five or six different types of beads that emit different colors in response to a laser excitement. In some embodiments, the fluorescent beads emit fluorescent light of one or more wavelengths in response to laser excitement. In some embodiments, the sample comprises a test target. In some embodiments, the test target comprises a coating of predetermined geometric shapes or patterns that are spatially repeated. In some embodiments, the predetermined geometric patterns or shapes are repeated in one or two dimensions. In some embodiments, the test target lacks a flow cell and a liquid. In some embodiments, the test target comprises one or more substrates with a predetermined refractive index. In some embodiments, the test target comprises a top substrate having a predetermined refractive index. In some embodiments, the test target comprises a bottom substrate. In some embodiments, at least a portion of the first or second substrates comprises the coating with the predetermined geometric patterns or shapes. In some embodiments, the thickness of the first substrate is configured to simulate the presence of a first hypothetical flow cell. In some embodiments, the thickness of the top substrate is configured to permit imaging of the bottom surface of the first channel of the hypothetical first flow cell. In some embodiments, the coating of the predetermined geometric shapes or patterns comprises optically opaque portions and transparent portions. In some embodiments, the optical system comprises 1, 2, 3, 4, 5, or 6 detection channels. In some embodiments, the optical system is configured to acquire flow cell images with a FOV of greater than 1.0 mm² after autofocusing of the optical system. In some embodiments, the optical system comprises: the objective lens; the image sensor; and a numerical aperture (NA) of less than 0.6; and the processor configured to process the flow cell images to correct for optical aberration and generate an optical resolution that is about identical in the flow cell images. In some embodiments, the optical system further comprises one or more illumination sources, wherein the one or more illumination sources lack an AF laser configured only for autofocusing of the optical system. In some embodiments, the method for autofocus of the optical system is configured for focusing the optical system for imaging the sample nucleic acid molecules immobilized to the flow cell in a first flow cycle and a second flow cycle in the sequencing run. In some embodiments, the method for autofocus of the optical system is configured for focusing the optical system for imaging the sample nucleic acid molecules immobilized to a first surface in a first flow cycle in a sequencing run, and for refocusing the optical system for imaging the sample nucleic acid molecules immobilized to the first surface or to a second surface in a second flow cycle in the sequencing run. In some embodiments, the method for autofocus of the optical system is configured for focusing the optical system for imaging the sample nucleic acid molecules immobilized to a first surface in a first flow cycle in a sequencing run, and for refocusing the optical system for imaging the sample nucleic acid molecules immobilized to a second surface in the first flow cycle or a second flow cycle in the sequencing run. In some embodiments, the method for autofocus of the optical system is configured for focusing at least along a z axis. In some embodiments, tilting the sample stage of the optical system by the tilt angle comprises: tilting the sample stage of the optical system by the tilt angle simultaneously as moving the sample stage within the x-y plane. In some embodiments, moving the sample stage within the x-y plane comprises moving the sample stage to a predetermined spatial location. In some embodiments, de-tilting the tilted sample stage by the tilt angle comprises: de-tilting the tilted sample stage by the tilt angle simultaneously as moving the sample stage relative to the focal plane of the objective lens by the determined z shift. In some embodiments, de-tilting the tilted image sensor by the tilt angle comprises: de-tilting the tilted image sensor by the tilt angle simultaneously as moving the sample stage by the determined z shift. In some embodiments, de-tilting the tilted AF sensor by the tilt angle comprises: de-tilting the tilted AF sensor by the tilt angle simultaneously as moving the sample stage by the determined z shift. In some embodiments, an error in autofocusing the optical system is in the range from −400 nm to +400 nm. In some embodiments, an error in autofocusing the optical system is in the range from −100 nm to +100 nm. In some embodiments, an error in autofocusing the optical system is in the range from −50 nm to +50 nm. In some embodiments, the sample stage is a motorized stage that automatically tilts by a predetermined angle provided by a user. In some embodiments, the image sensor or the AF sensor is immobilized on a motorized stage that automatically tilts by a predetermined angle provided by a user. In some embodiments, the objective lens is immobilized on a z-stage that is movable along the z-axis. In some embodiments, moving the sample stage relative to a focal plane of an objective lens of the optical system by the determined z shift comprises moving the objective lens thereby moving the focal plane of the objective lens by the determined z shift. In some embodiments, tilting the sample stage of the optical system by the tilt angle comprises: receiving, by a motor coupled to the sample stage, the tilt angle; and tilting, by the motor, the sample stage by the tilt angle.

The present disclosure provides for a method for autofocus of an optical system, comprising: acquiring, by the optical system, one or more flow cell images of a first tile or subtile of the sample in a flow cycle of a sequencing run; moving the sample stage to position a second tile or subtile next to the first tile or subtile sample relative to the optical system; repeating the method for autofocusing of the optical system; and acquiring, by the optical system, one or more flow cell images of the second tile or subtile of the sample in the flow cycle of the sequencing run.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The novel features of the inventive concepts are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a block diagram of a next generation sequencing (NGS) system utilizing an optical system disclosed herein for imaging sequencing reactions and for sequencing analysis, in accordance with some embodiments.

FIGS. 2A-2B illustrate a non-limiting example of an optical system comprising a dichroic beam splitter for transmitting an excitation light beam to a sample, and for receiving and redirecting by reflection the resultant fluorescence emission to four detection channels configured for detection of fluorescence emission at four different respective wavelengths or wavelength bands. FIG. 2A: top isometric view. FIG. 2B: bottom isometric view.

FIGS. 3A-3B illustrate the optical paths within the optical system of FIGS. 2A and 2B comprising a dichroic beam splitter for transmitting an excitation light beam to a sample, and for receiving and redirecting by reflection a resultant fluorescence emission to four detection channels for detection of fluorescence emission at four different respective wavelengths or wavelength bands. FIG. 3A: top view. FIG. 3B: side view.

FIG. 4 illustrates a block diagram of a computer system for autofocus of the optical system, in accordance with some embodiments.

FIG. 5 shows a flow chart of an example of an image-based autofocusing method of the optical system, in accordance with some embodiments.

FIG. 6A shows a schematic view of tilting the sample stage relative to the image sensor and determining the z shift for autofocus of the optical systems, in accordance with embodiments herein.

FIG. 6B shows a non-limiting example image that is used to determine the x-y plane shift for autofocus of the optical system, in accordance with embodiments herein.

FIGS. 7A-7B shows autofocusing results using the methods and systems herein by tilting the sample stage with different tilt angles in comparison with reference z shifts, in accordance with some embodiments.

FIG. 8 shows autofocusing results using the methods and systems herein with tilting the image sensor in comparison with reference z shifts, in accordance with some embodiments.

FIG. 9 shows a calibration image that is used to determine a pivot point offset before autofocusing of the optical system using the methods and systems herein, in accordance with some embodiments.

FIG. 10 shows a schematic view of the sample immobilized on the sample stage and their positions relative to the objective lens along the optical axis of the optical system, in accordance with some embodiments.

FIG. 11 shows a schematic illustration of an example of an embodiment of the low binding solid supports, in which the support comprises a glass substrate and alternating layers of hydrophilic coatings which are covalently or non-covalently adhered to the glass, and which further comprises chemically-reactive functional groups that serve as attachment sites for oligonucleotide primers.

FIG. 12 is a schematic of various example configurations of multivalent molecules. Left (Class I): schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’.

FIG. 13 is a schematic of an example of a multivalent molecule comprising a generic core attached to a plurality of nucleotide-arms.

FIG. 14 is a schematic of an example of a multivalent molecule comprising a dendrimer core attached to a plurality of nucleotide-arms.

FIG. 15 shows a schematic of an example of a multivalent molecule comprising a core attached to a plurality of nucleotide-arms, where the nucleotide arms comprise biotin, spacer, linker and a nucleotide unit.

FIG. 16 is a schematic of an example of a nucleotide-arm comprising a core attachment moiety, spacer, linker and nucleotide unit.

FIG. 17 shows the chemical structure of an example of a spacer (top), and the chemical structures of various examples of linkers, including an 11-atom Linker, 16-atom Linker, 23-atom Linker and an N3 Linker (bottom).

FIG. 18 shows the chemical structures of various examples of linkers, including Linkers 1-9.

FIG. 19 shows the chemical structures of various examples of linkers joined/attached to nucleotide units.

FIG. 20 shows the chemical structures of various examples of linkers joined/attached to nucleotide units.

FIG. 21 shows the chemical structures of various examples of linkers joined/attached to nucleotide units.

FIG. 22 shows the chemical structures of various examples of linkers joined/attached to nucleotide units.

FIG. 23 shows the chemical structure of an example of a biotinylated nucleotide-arm. In this example, the nucleotide unit is connected to the linker via a propargyl amine attachment at the 5 position of a pyrimidine base or the 7 position of a purine base.

FIG. 24 shows an example of an embodiment of the test target disclosed herein.

FIG. 25 shows a schematic drawing of an example of a flow cell having a first surface coated with fluorescent beads (top) and a second surface coated with fluorescent beads (bottom). The coating can be directly applied on the solid support of the flow cell. The flow cell with the coating(s) can be positioned on a sequencing system for autofocusing of the optical system by obtaining and analyzing images of the fluorescent beads.

### DETAILED DESCRIPTION

There is a need for accurate and reliable autofocus of multi-channel fluorescence imaging systems to ensure quality of fluorescent images and accuracy in sequencing analysis based thereupon. Disclosed herein are systems and methods that may provide one or more of the following advantages. The systems and methods herein advantageously remove the need for dedicated AF hardware such as an AF illumination source, AF sensor, and/or AF tube lens, so that the machinery cost and the complexity of the imaging system is reduced. Compared with image-based AF methods using multiple images and/or machine learning algorithms, the systems and methods herein only require a single image, which reduces time consumption and computational complexity in achieving autofocusing. Further, the systems and methods herein reduce the level of photo bleaching in existing autofocus methods using dedicated AF hardware, since the systems and methods herein avoid acquiring multiple images after illumination at multiple z locations. More importantly, the systems and methods described herein can achieve an error range of less than 100 nm in AF, which is comparable or improved over existing AF methods. The tilting and de-tilting of sample stage or sensor used in the methods disclosed herein can be simultaneously performed with other preparation operations for imaging, e.g., moving the x-y stage or the objective lens relative to each other to position a desired area of the flow cell for imaging, to save the total time needed to achieve autofocusing and imaging. The total time for autofocusing using the systems and methods herein may be completed in less than 500 milliseconds, making it feasible for repeated use in each flow cycle in various sequencing applications.

Although the methods and systems described herein are disclosed in the context of multi-channel fluorescence imaging systems for DNA sequencing applications, they may be utilized for autofocus of various optical systems in different applications that require z-axis autofocus to render in-focus images.

### Sequencing Systems

The optical systems disclosed herein can be utilized in various applications that utilizes in-focus images containing optical signals, for example, in next generation sequencing (NGS) sequencing applications or as part of a NGS sequencing system.

In some embodiments, the optical system disclosed herein can include some or all of the optical elements of a multi-channel fluorescent imaging module of a NGS sequencing system.

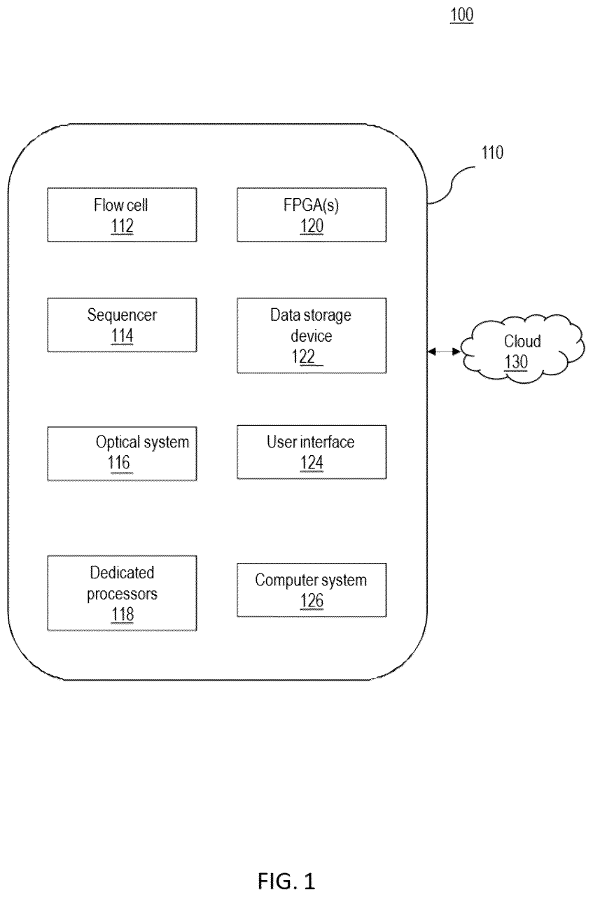

FIG. 1 illustrates a block diagram of a computer-implemented system 100 that is configured to perform DNA sequencing and sequencing analysis, according to one or more embodiments disclosed herein. The system 100 can have a sequencing system 110 that includes a flow cell 112 or a test target that simulates the presence of the flow cell, a sequencer 114, an optical system 116, data storage 122, and a user interface 124. The sequencing system 110 may be connected to a cloud 130. The sequencing system 110 may include one or more of dedicated processors 118, Field-Programmable Gate Array(s) (FPGAs) 120, and a computer system 126.

In some embodiments, the flow cell or test target that simulates the presence of the flow cell may be used in autofocusing of the sequencing systems. In some embodiments, the image that can be used in autofocusing of the sequencing systems may be generated by collecting optical signals emitted from the flow cell or test target that simulates the presence of the flow cell.

In some embodiments, the flow cell may have traditional 2D DNA samples immobilized thereon. In some embodiments, the flow cell may have volumetric 3D samples immobilized thereon. The 3D samples can include in situ cells and/or tissues.

In some embodiments, the sample herein may include unbalanced or balanced nucleotide acids in one or more flow cycles.

In some embodiments, the flow cell 112 is configured to capture DNA fragments and form DNA sequences for base-calling on the flow cell. The flow cell or test targe herein 112 may include the support as disclosed herein. The support may be a solid support. The support may include a surface coating thereon as disclosed herein. The surface coating may be a polymer coating as disclosed herein. A flow cell 112 may include multiple tiles or imaging areas thereon, and each tile may be separated into a grid of subtiles. Each subtile may include a plurality of clusters or polonies thereon.

In some embodiments, the flow cell may comprise more than one substrate. The flow cell may comprise interior surfaces that are separated by a fluid channel through which an analyte or reagent can flow. In some embodiments, the flow cell may comprise at least two, three, four, five, six, or even more interior surfaces that are separated by corresponding fluid channels through which an analyte or reagent can flow. In such embodiments, autofocusing and imaging can occur at each individual interior surface for various sequencing applications. Having multiple interior surfaces with sequencing reactions that can be imaged can advantageously increase the throughput of sequencing than using traditional flow cell with only one or two interior surfaces.

In some embodiments, the flow cell may comprise one or more surfaces and one or more substrates. The flow cell may comprise at least one hydrophilic polymer coating layer and a plurality of oligonucleotide molecules attached to at least one hydrophilic polymer coating layer. In some embodiments, the flow cell may include at least one discrete region of the one or more surfaces that comprises a plurality of clonally-amplified, sample nucleic acid molecules immobilized to the plurality of attached oligonucleotide molecules. The sample nucleic acid molecules, when being imaged, shows up as bright spots or “polonies” of signals.

In some embodiments, the flow cell may be a beaded flow cell that includes patterned or randomly distributed fluorescent or luminescent beads. In some embodiments, the flow cell includes a beaded flow cell with randomly distributed microbeads with fluorescent label to simulate fluorescent light emission from DNA samples upon illumination by the illumination source disclosed herein. In some embodiments, the fluorescent beads can be microbeads that are commercially available. In some embodiments, the microbeads are customized. In some embodiments, the beaded flow cell comprises a surface coated with fluorescent beads that are chemically immobilized to the surface. The fluorescent beads may comprise one, two, three, four, five or six different types of beads that emits different colors and/or light at different frequencies in response to an optical excitement, e.g., a laser light. The fluorescent beads may emit fluorescent light of one or more wavelengths in response to laser excitement.

In some embodiments, a flow cell device disclosed herein can comprise a support disclosed herein. The support can be solid. At least part of the support can be transparent. The support can comprise one or more substrates. At least part of the one or more substrate can be transparent. FIG. 25 shows an example embodiment of the flow cell device 900. The flow cell device 900 includes a support 901 and other flow cell compounds such as coatings. The support can comprise a top substrate 910 and a bottom substrate 910. Each substrate 910 can have a predetermined thickness, and different substrates can have different thicknesses. The substrate can define one or more channels 920 of the device 900. The channels can allow fluid flow therethrough, e.g., liquid or air. The flow cell device can include one or more inlet 920 and one or more outlets 930 in the one or more substrate 910.

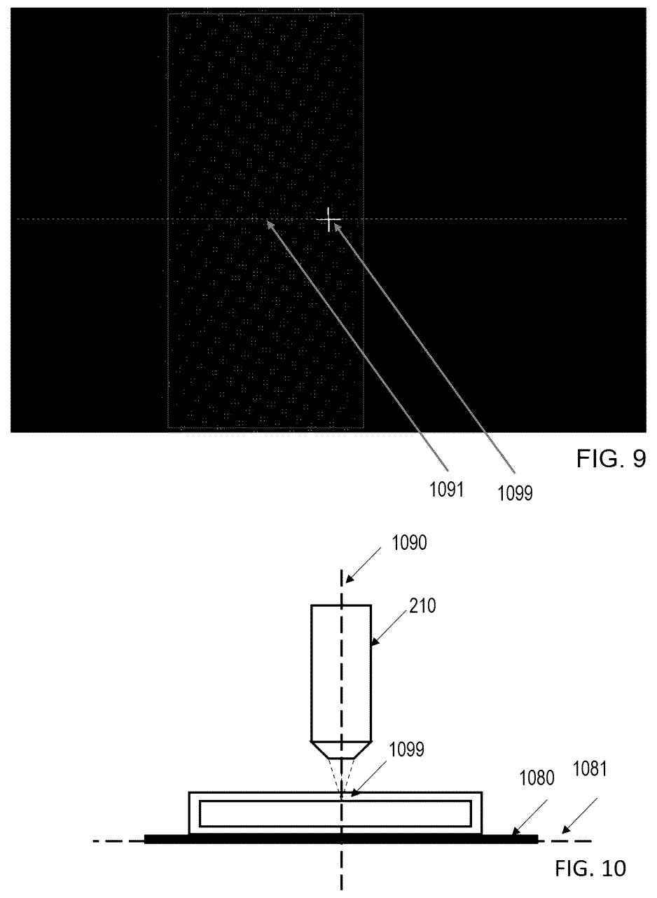

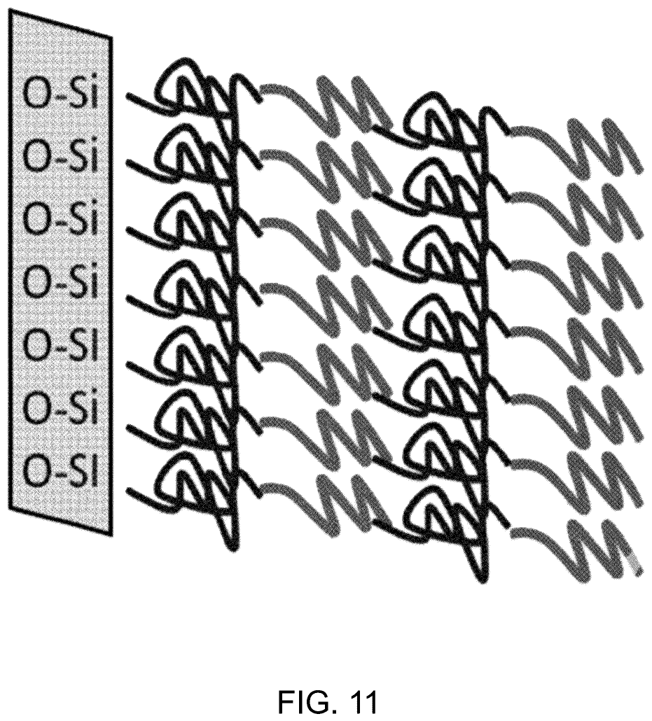

FIGS. 9 and 11 show an example device 900 with two substrates forming two channels, and each channel having an inlet and an outlet. However, the number of substrates, channels, inlets and outlets in other embodiments can be different. In some embodiments, the number of substrates, channels, inlets and outlets can be any integer number that is greater than 0. FIG. 25 shows an example flow cell 900 with two planar substrates without curvature on the surface(s) of the substrate. However, the substrate does not have to be planar.

In some embodiments, the support and the one or more substrates can comprise glass or plastic. In some embodiments, one or more substrates are all-glass or all-plastic.

In some embodiments, the one or more channels 920 can run from the inlet 930 to the outlet 940 so that fluid can flow from the inlet 930 via the one or more channels 920 to the outlet 940. As an example, sequencing reagents can be introduced to the flow cell device via the inlet, flow through the channels, and then exit from the outlet. The channel(s) 920 can comprise a top interior surface 921 and a bottom interior surface 922. One or more of the surfaces can be coated with fluorescent beads.

The fluorescent beads can be chemically immobilized to the surface. The fluorescent beads can be covalently immobilized to the surface. The fluorescent beads can be immobilized or fixedly attached to the surface 921, 922 by forming a coating 950, 951 thereon, so that the fluorescent beads remain fixed or immobilized relative to the surface 921, 922. The coating 950, 951 can be applied directly to and in contact with the interior surface 921, 922. Alternatively, the coating 950, 951 can be applied indirectly to or not in direct contact with the interior surface 921, 922. In some embodiments, the coating 950, 951 can be applied with some compounds in between the surface 921, 922 and the coating 950, 951. For example, the coating 950, 951 can be applied on top of another coating that is directly applied to and in contact with the surface 921, 922. In some embodiments, the surface is passivated with another coating (not shown). The another coating can immobilize surface capture primers, nucleic acid template molecules, or both for capturing polynucleotides on the surface 921, 922. In some embodiments, the surface 921, 922 comprises polynucleotides captured thereon.

In some embodiments, the coating 951, 952 that attaches the fluorescent beads can be mixed with one or more other coatings so that the mixed coating can be applied directly to and in contact with the interior surface 921, 922. The mixed coating can immobilize fluorescent beads on the surface. Further, the mixed coating may also immobilize surface capture primers, nucleic acid template molecules, or both for capturing polynucleotides on the surface 921, 922. In some embodiments, the fixed coating may capture polynucleotides on the surface 921, 922, and administration of sequencing reagents can facilitate sequencing of the polynucleotides as disclosed herein using various sequencing methods, for example, sequencing-via-avidite.

In some embodiments, the flow cell device 900 can be used on a sequencing system 1410 for DNA sequencing. The flow cell device 900 may receive various sequencing reagents before a sequencing cycle via the inlet 930 and allow the reagent(s) to flow through the one or more channels 920 and exit via the outlet 940. In some embodiments, the fluorescent beads remain immobilized relative to the surface 921, 922 during or after administration of sequencing reagents to the flow cell device 900.

In some embodiments, the flow cell or beaded flow cell may include a sample immobilized thereon such as nucleic acid molecules tethered on a substrate of the flow cell.

In some embodiments, the test target comprise substrates with a gap or another substrate therebetween to simulate the fluidic channel with liquid.

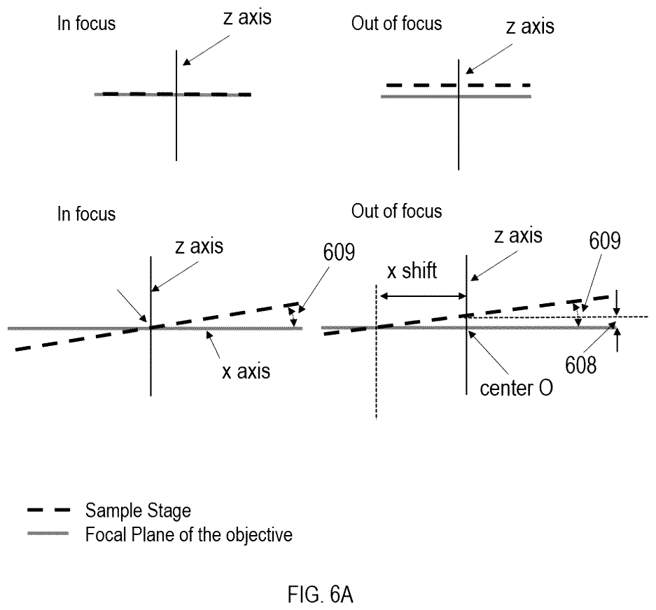

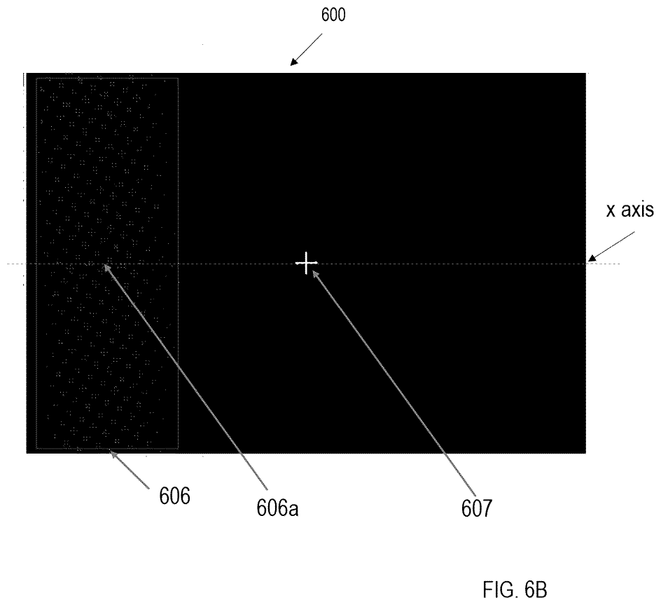

In some embodiments, the test target comprises a coating of predetermined geometric shapes or patterns. In some embodiments, the predetermined geometric patterns or shapes are spatially repeated in one or two dimensions. For example, the test target may comprise a grid of intersecting lines. As another example, the test target may comprise micro-dots that are separated by identical distances in 2D. FIG. 6B and FIG. 9 show repeated geometric patterns of an example test target. In some embodiments, the test target lacks a flow cell and a liquid. In some embodiments, the test target comprises one or more substrates with a predetermined refractive index. In some embodiments, the test target comprises a top substrate having a predetermined refractive index of [n-top substrate(1)]. In some embodiments, the test target comprises a bottom substrate. At least a portion of the first or second substrates may comprise the coating of the predetermined geometric patterns or shapes. In some embodiments, the thickness of the first substrate is configured to simulate the presence of a first hypothetical flow cell, wherein the first hypothetical flow cell includes a first channel having a top surface and bottom surface, and the first channel containing a designated first fluid, wherein the first channel has a first designated thickness of [T-channel(1)] and the first designated fluid has a refractive index of [n-fluid(1)]. In some embodiments, the thickness of the top substrate is configured to permit imaging of the bottom surface of the first channel of the hypothetical first flow cell. In some embodiments, the coating of the predetermined geometric shapes or patterns comprises optically opaque portions and transparent portions. In some embodiments, the height or thickness of the top substrate [T-top substrate(1)] depends on the refractive index of the top substrate [n-top substrate(1)], the first designated height of the first channel [T-channel(1)] and the refractive index of the first designated fluid [n-fluid(1)]. In some embodiments, the height or thickness of the top substrate [T-top substrate(1)] can be calculated as:

$$( T - top substrate ( 1 ) ) = C * [ ( ( T - channel ( 1 ) ) * [ ( ( n - fluid ( 1 ) ) / ( n - top substrate ( 1 ) ) ] ]$$

where C is a constant that can be predetermined.

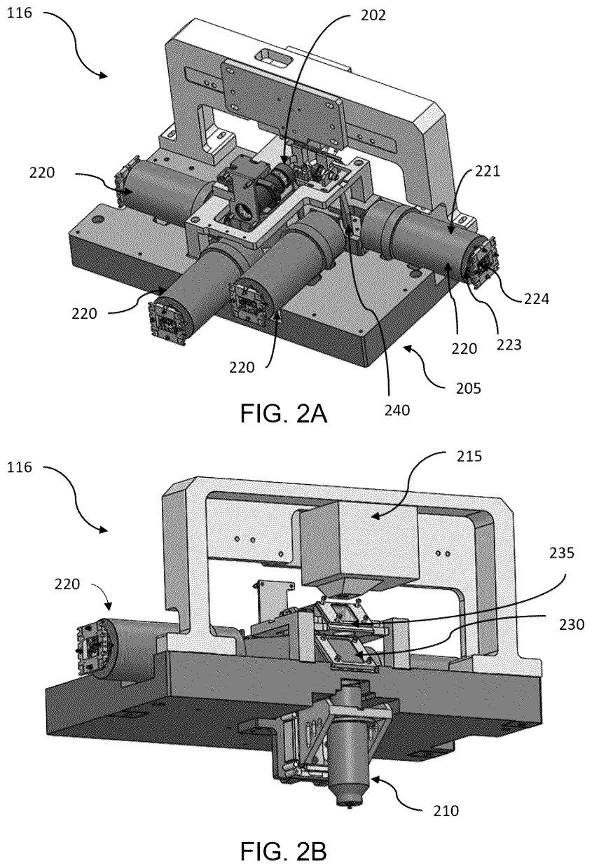

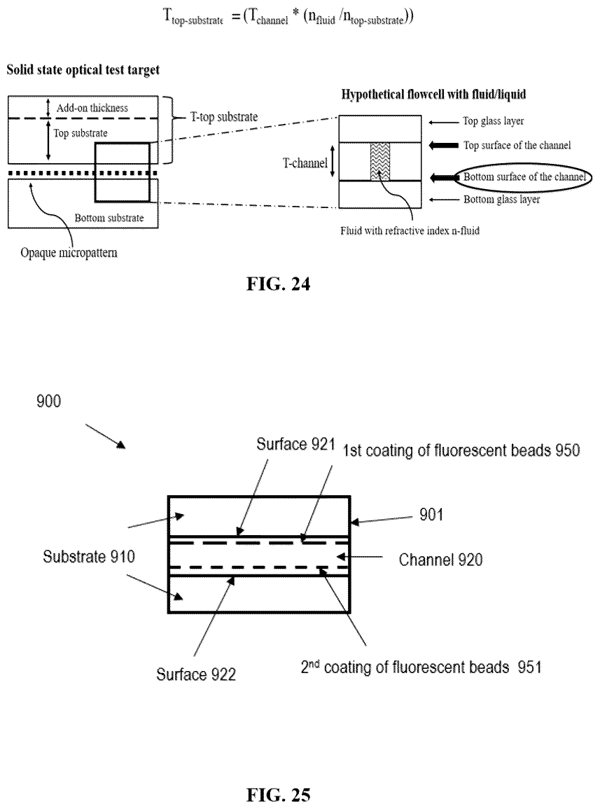

FIG. 24 is a schematic of an example embodiment of the test target herein. The left schematic shows an example solid state optical test target having a first substrate (top) and second substrate (bottom) with an opaque layer between the first and second substrate. The opaque layer can be coated on the bottom surface of the first substrate or the top surface of the second substrate. The opaque layer forms a micropattern. The first substrate is transparent which permits light transmission from its bottom surface and a view of the micropattern. The solid state optical test target lacks a flow cell and liquid. The thickness of the first substrate is adjusted to simulate the presence of a hypothetic flow cell which contains a fluid/liquid, where the hypothetical flow cell may be located between the first and second substrates. For example, the first substrate is thicker, having an add-on thickness. The right schematic shows a hypothetical flow cell which includes a channel having a thickness [T-channel] and the channel contains a fluid/liquid having a refractive index [n-fluid]. The solid state optical test target shown in FIG. 2 can be positioned on an optical imaging system and used to evaluate the performance of the optical imaging system by obtaining image information about the bottom surface of the hypothetical flow cell channel.

The sequencer 114 may be configured to flow a nucleotide mixture onto the flow cell 112, cleave blockers from the nucleotides in between flowing operations, and perform other operations for the formation of the DNA sequences on the flow cell 112. The nucleotides may have fluorescent elements attached that emit light or energy in a wavelength that indicates the type of nucleotide. Each type of fluorescent element may correspond to a particular nucleotide base (e.g., A, G, C, T). The fluorescent elements may emit light in visible wavelengths. In some aspects, the sequencer 114 and the flow cell 112 may be configured to perform various sequencing methods, for example, sequencing-by-avidity. For example, each nucleotide base may be assigned a color. Different types of nucleotides may have different colors. Adenine (A) may be red, cytosine (C) may be blue, guanine (G) may be green, and thymine (T) may be yellow, for example. The color or wavelength of the fluorescent element for each nucleotide may be selected so that the nucleotides are distinguishable from one another based on the wavelengths of light emitted by the fluorescent elements.

A test target may be used to simulate the presence of a hypothetical flow cell. It may include similar fluorescent signals as a flow cell, e.g., of a similar wavelength, originating from areas that are of comparable size to polonies or clusters on a flow cell, and/or of comparable intensity to the signals from actual samples immobilized on flow cells.

The optical system 116 may be focused using the autofocus methods herein. The optical system 116 may be configured to capture images of the flow cell or test target after autofocusing. In some embodiments, the image sensor of the optical system 116 or the optical system can include a camera configured to capture digital images, such as an active-pixel sensor (CMOS) or a CCD camera. The image sensor may be configured to capture images at the wavelengths of the fluorescent elements bound to the nucleotides. The images may be called flow cell images. The images may then be used for base calling.

In some embodiments, the images of the flow cell or test target may be captured in one or more color channels, where each image in the channel is taken at a wavelength or in a wavelength spectrum that matches or includes mostly one type of the fluorescent elements. In some other embodiments, the images may be captured as images that capture all of the wavelengths of the fluorescent elements.

The resolution of the optical system 116 controls the level of detail in the flow cell images, including pixel size. In existing systems, this resolution is very important, as it controls the accuracy with which a spot-finding algorithm identifies the polony centers. One way to increase the accuracy of spot finding is to improve the resolution of the optical system 116 (e.g., by incorporating a higher-resolution camera), or to improve the processing performed on images taken by the optical system 116. Detecting polony centers in pixels other than those detected by a spot-finding algorithm may be performed. These processing-based methods may allow for improved accuracy in detection of polony centers without increasing the resolution of the optical system 116. The resolution of the optical system may even be less than existing systems with comparable performance, which may reduce the cost of the sequencing system 110. In some aspects, the resolution of the optical system may be the same as existing systems but achieve superior performance as compared to those existing systems due to the image processing.

The image quality of the flow cell images controls the base calling quality. One way to increase the accuracy of base calling is to improve the optical system 116, or improve the processing performed on images taken by optical system 116 to result in a better image quality. The methods described herein enables AF that can be conveniently and efficiently performed whenever needed, e.g., before or during a sequencing run. In some embodiments, the methods described herein may be advantageously performed before imaging a flow cell, and such AF may be repeated as needed during the sequencing run.

The optical system 116 may be configured to perform autofocusing before imaging, e.g., in each flow cycle of a sequencing run. The operations or actions disclosed herein may be performed by the dedicated processors 118, the FPGA(s) 120, the computing system 126, or a combination thereof. One or more operations or actions in methods 500 disclosed herein may be performed by the dedicated processors 118, the FPGA(s) 120, the computing system 126, or a combination thereof. In some embodiments, which operations or actions are to be performed by the dedicated processors 118, the FPGA(s) 120, the computing system 126, or combinations thereof may be determined based on one or more of: a computation time for the specific operation(s), the complexity of the computation in the specific operation(s), the need for data transmission between the hardware devices, or combinations thereof.

The computing system 126 may include one or more general purpose computers or hardware processors that provide interfaces to run a variety of program in an operating system, such as Windows™ or Linux™. Such an operating system typically provides great flexibility to a user.

In some aspects, the dedicated processors 118 may be configured to perform operations in the methods herein. They may not be general-purpose processors, but instead custom processors with specific hardware or instructions for performing those operations. Dedicated processors directly run specific software without an operating system. The lack of an operating system reduces overhead, at the cost of the flexibility in what the processor may perform. A dedicated processor may make use of a custom programming language, which may be designed to operate more efficiently than the software run on general-purpose computers. This may increase the speed at which the operations are performed and allow for real time processing.

In some aspects, the FPGA(s) 120 may be configured to perform operations disclosed herein. An FPGA is programmed as hardware that will only perform certain specific tasks. A special programming language may be used to transform software operations into hardware componentry. Once an FPGA is programmed, the hardware directly processes digital data that is provided to it without running software. Instead, the FPGA uses logic gates and registers to process the digital data. Because there is no overhead required for an operating system, an FPGA generally processes data faster than a general-purpose computer. Similarly to dedicated processors, this is at the cost of flexibility. The lack of software overhead may also allow an FPGA to operate faster than a dedicated processor, although this will depend on the exact processing to be performed and the specific FPGA and dedicated processor.

In some aspects, the data storage 122 is used to store information used in the optical alignment methods. This information may include the images themselves or information derived from the images (e.g., pixel intensities, colors, etc.) captured by the optical system 116.

The user interface 124 may be used by a user to operate the sequencing system or access data stored in the data storage 122 or the computer system 126.

The computer system 126 may control the general operation of the sequencing system and may be coupled to the user interface 124. It may also perform operations disclosed herein for optical alignment. The computer system 126 may store information regarding the operation of the sequencing system 110, such as configuration information, instructions for operating the sequencing system 110, or user information. The computer system 126 may be configured to pass information between the sequencing system 110 and the cloud 130.

As discussed above, the sequencing system 110 may have dedicated processors 118, FPGA(s) 120, or the computer system 126. The sequencing system may use one, two, or all of these elements to accomplish necessary processing described above. In some aspects, when these elements are present together, the processing tasks are split between them.

The cloud 130 may be a network, remote storage, or some other remote computing system separate from the sequencing system 110. The connection to cloud 130 may allow access to data stored externally to the sequencing system 110 or allow for updating of software in the sequencing system 110.

### Optical Systems

In some embodiments, the AF methods and systems described herein may be utilized for autofocus of various optical systems. The various optical systems may be used in different applications that require z-axis autofocus to render in-focus images. In some embodiments, the AF methods and systems described herein may be, but are not limited to, utilized for autofocus of the optical systems described herein.

In some embodiments, the AF methods and systems may be utilized for autofocusing of optical systems or optical assemblies whose details are disclosed in PCT Patent Application No. PCT/US2024/012802, and are incorporated herein by reference in its entirety.

The sequencing systems (e.g., 100 in FIG. 1) disclosed herein may include an optical system 116. In some embodiments, the optical system 116 is a multi-channel imaging module. In some embodiments, the multi-channel imaging module can comprise: one or more illumination sources; an objective lens shared by multiple detection channels; a sample immobilized on a flow cell or a test target; a sample stage configured to hold the test target or the flow cell thereon; a numerical aperture within a predetermined range, a processor configured to determine z shift for autofocusing; or combinations thereof. Each detection channel can comprise: a corresponding tube lens and a corresponding image sensor. The sample stage and/or the image sensor may be motorized or mounted to a motorized stage so that they are tiltable by a tilt angle provided by a user.

FIGS. 2A and 2B illustrate a non-limiting example of the optical system 116 disclosed herein. The optical system 116 can include an objective lens 210, one or more illumination sources 215, and one or more detection channels 220.

The optical system 116 may also include one or more dichroic filters 230, 235, 240, which may comprise a dichroic reflector or beam splitter.

As shown in FIGS. 2A-2B, the optical system 116 may comprise hardware that is used for autofocusing only but not for imaging purposes. Such hardware can include but is not limited to one or more of: one or more AF illumination sources, an AF sensor 202, an AF tube lens, and a dichroic filter or beam splitter. The one or more AF illumination sources may include an AF laser, for example, one which projects a spot the size of which is monitored to determine when the optical system is in-focus. FIGS. 2A and 2B also show a dichroic filter 235, which may comprise for example a dichroic beam splitter or beam combiner, which may be used to direct the autofocus laser through the objective and to the sample support structure.

In some embodiments, the AF sensor may be tiltable by the tilt angle disclosed herein. The AF sensor may be connected to a motor or a hexapod so that the AF sensor may be tilted automatically in response to receiving an instruction by a user or a computer system.

In some embodiments, the optical system 116 may comprise hardware that is configured both for autofocusing and imaging purposes. In some embodiments, the optical system 116 may not comprise any hardware that is only used for autofocusing. In other words, hardware in the optical system 116 can be used either for both autofocusing and imaging purposes, or only for imaging purposes. Such hardware only for autofocusing include one or more of: an AF illumination source, an AF sensor 202, an AF tube lens, and a dichroic filter or beam splitter. In some embodiments, the optical system 116 lacks an AF illumination source and an AF sensor. In some embodiments, the optical system that lacks dedicated hardware for AF purpose may look identical to that in FIGS. 2A-2B, except that the dedicated AF laser and AF sensor 202 are removed. In some embodiments, the dichroic filter 235 may also be removed because it works in directing illumination to the AF sensor.

Some or all components of the optical system 116 may be coupled to a baseplate 205, either fixedly or movably. The objective lens 210 may be fixedly coupled to a z-stage that is movable relative to the base plate 205. The z-stage can move along the optical axis 1090 or z-axis of the optical system. The z-stage can be a motorized stage, wherein its movement can be automatic after receiving an instruction or input either from a user or a computer system disclosed herein.

The optical axis of the optical system is shown in FIG. 10. As disclosed herein, the optical axis is used equivalent as the z-axis here. The optical axis may be a straight line that passes through the geometrical center of the objective lens and the geometrical center of the field of view being imaged in the sample. In some embodiments, the optical axis may be a straight line that passes through the geometrical center of each image sensor of the optical system. In some embodiments, a center of an image acquired using the optical system is along the optical axis.

In some embodiments, the optical system 116 can include a sample stage for holding a test target or a sample support, e.g., a flow cell, with a sample immobilized thereon. The sample stage may be positioned next to the objective lens along the optical axis 1190 of the optical system. In some embodiments, the sample stage may be motorized or mounted to a motorized stage so that it is tiltable by a tilt angle. In some embodiments, the sample stage may be movable (translatable and/or tiltable) in three dimensions (3D). In some embodiments, the sample stage may be movable in 3D relative to the objective lens. FIG. 10 shows an example sample stage 1080 that is motorized and has a flow cell immobilized thereon. The flow cell may include multiple tiles or subtiles. The sample stage may be moved so that the geometrical center 1099 of a specific tile or subtile is at the optical axis 1090 when the corresponding tile or subtile is being imaged. The sample stage may move within the x-y plane 1081, e.g., the image plane.

As disclosed herein, the tilt angle for tilting any optical element of the optical system 116, e.g., the sample stage, the image sensor, etc., may be about any axis in 3D. In some embodiments, the tilt angle is about the x or y axis. In some embodiments, the tilt angle is within a x-z plane or y-z plane. In some embodiments, the tilt angle may be determined based on the sample size, the size of the image sensor, and/or combinations thereof. In some embodiments, the tilt angle is large enough so that the entire in-focus region is within the FOV of the image 600. In some embodiments, the tilt angle is smaller than a threshold so that the in-focus region includes at least a certain number of pixels in its smallest dimension, e.g., along the x axis as shown in FIG. 6B.

In some embodiments, the tilt angle is in a range from 0.01 degrees to 89.9 degrees. In some embodiments, the tilt angle is in a range from 0.05 degrees to 15 degrees. In some embodiments, the tilt angle is in a range from 0.05 degrees to 5 degrees. In some embodiments, the tilt angle is clockwise about the x or y axis. In some embodiments, the tilt angle is counter-clockwise about the x or y axis. In some embodiments, the tilt angle is clockwise in the z-x or y-z plane. In some embodiments, the tilt angle is counter-clockwise in the z-x or y-z plane.

The optical system may comprise one or more illumination sources 215. In some embodiments, the illumination source 215 lacks any AF illumination source that is only used for autofocusing purposes. The AF illumination source may include one or more AF lasers that are configured only for autofocusing purposes. In some embodiments, the illumination source 215 herein is used for both autofocusing and for imaging after autofocusing. In some embodiments, the illumination source 215 comprises a laser.

The illumination source 215 may include any suitable light source configured to produce light of a predetermined excitation wavelength(s). The light source may be a broadband source that emits light within one or more excitation wavelength ranges (or bands). The light source may be a narrowband source that emits light within one or more narrower wavelength ranges. In some embodiments, the light source may produce a single isolated wavelength (or line) corresponding to the desired excitation wavelength, or multiple isolated wavelengths (or lines). In some embodiments, the lines may have some very narrow bandwidth. Example light sources that may be suitable for use in the illumination source 215 include, but are not limited to, an incandescent filament, xenon arc lamp, mercury-vapor lamp, a light-emitting diode, a laser source such as a laser diode or a solid-state laser, or other types of light sources. As discussed below, in some designs, the light source may comprise a polarized light source such as a linearly polarized light source. In some embodiments, the orientation of the light source is such that s-polarized light is incident on one or more surfaces of one or more optical components such as the dichroic reflective surface of one or more dichroic filters.

The illumination source 215 may further include one or more additional optical components such as lenses, filters, optical fibers, or any other suitable transmissive or reflective optics as appropriate to output an excitation light beam having suitable characteristics toward the dichroic filter 230. For example, beam shaping optics may be included, for example, to receive light from a light emitter in the light source and produce a beam and/or provide a desired beam characteristic. Such optics may, for example, comprise a collimating lens configured to reduce the divergence of light and/or increase collimation and/or to collimate the light.

In some embodiments, multiple light sources are included in the optical system 116. In some such embodiments, different light sources may produce light having different spectral characteristics, for example, exciting different fluorescence dyes. In some embodiments, light produced by the different light sources may be directed to coincide and form an aggregate excitation light beam. This composite excitation light beam may be composed of excitation light beams from each of the light sources. The composite excitation light beam will have more optical power than the individual beams that overlap to form the composite beam. For example, in embodiments that include two light sources that produce two excitation light beams, the composite excitation light beam formed from the two individual excitation light beams may have optical power that is the sum of the optical power of the individual beams. Similarly, in some embodiments, three, four, five or more light sources may be included, and these light sources may each output excitation light beams that together form a composite beam that has an optical power that is the sum of the optical power of the individual beams.

In some embodiments, the light source 215 outputs a sufficiently large amount of light to produce sufficiently strong fluorescence emission. Stronger fluorescence emission can increase the signal-to-noise ratio (SNR) and the contrast-to-noise ratio (CNR) of images acquired by the fluorescence imaging module. In some embodiments, the output of the light source and/or an excitation light beam derived therefrom (including a composite excitation light beam) may range in power from about 0.5 Watts to about 5.0 Watts, or more.

Referring again to FIGS. 2A and 2B, the dichroic filter 230 can be disposed with respect to the light source to receive light therefrom. The dichroic filter may comprise a dichroic mirror, dichroic reflector, dichroic beam splitter, or dichroic beam combiner configured to transmit light in a first spectral region (or wavelength range) and reflect light having a second spectral region (or wavelength range). The first spectral region may include one or more spectral bands, e.g., one or more spectral bands in the ultraviolet and blue wavelength ranges. Similarly, a second spectral region may include one or more spectral bands, e.g., one or more spectral bands extending from the green to red and infrared wavelengths. Other spectral regions or wavelength ranges are also possible.

In some embodiments, the dichroic filter 230 may be configured to transmit light from the light source to a sample support structure such as a microscope slide, a capillary, a flow cell, a test target, a microfluidic chip, or another substrate or support structure. The sample support structure supports and positions the sample, e.g., a composition comprising a fluorescently-labeled nucleic acid molecule or complement thereof, with respect to the optical system 116. In some embodiments, e.g., during optical alignment herein, the sample can be the test target comprising geometric shapes and/or patterns that simulate the presence of fluorescently-labeled nucleic acids. Accordingly, a first optical path extends from the light source to the sample via the dichroic filter 230. In various embodiments, the sample support structure includes at least one surface on which the sample is disposed or to which the sample binds. In some embodiments, the sample may be disposed within or bound to different localized regions or sites on the at least one surface of the sample support structure.

In some embodiments, the support structure may include two surfaces located at different distances from objective lens 210 (e.g., at different positions or depths along the optical axis of objective lens 210) on which the sample is disposed. As discussed below, for example, a flow cell may comprise a fluid channel formed at least in part by first and second (e.g., upper and lower) interior surfaces, and the sample may be disposed at localized sites on the first interior surface, the second interior surface, or both interior surfaces. The first and second surface may be separated by the region corresponding to the fluid channel through which a solution flows, and thus be at different distances or depth with respect to objective lens 210 of the optical system 116.

In some embodiments, the optical system 116 includes at least one objective lens 210. In some embodiments, the optical system 116 includes a single objective lens 210. The objective lens 210 may be shared by some or all of the detection channels. The objective lens may be mounted fixedly or movably to the baseplate 205. In some embodiments, the objective lens 210 is mounted fixedly to the baseplate 205. Movement of the baseplate 205 moves the objective lens correspondingly, for example, to focus the sample to the focal plane of the objective lens. In some embodiments, the objective lens 210 is mounted movably to the baseplate 205. Movement of the objective lens relative to the sample stage can be enabled by moving the objective lens 210 itself.

The objective lens 210 may be included in the first optical path between the dichroic filter 230 and the sample or the test target. This objective lens may be configured, for example, to have a focal length, working distance, and/or be positioned to focus light from the light source(s) onto the sample, e.g., onto a surface of the microscope slide, capillary, flow cell, microfluidic chip, or other substrate or support structure. Similarly, the objective lens 210 may be configured to have suitable focal length, working distance, and/or be positioned to collect light reflected, scattered, or emitted from the sample (e.g., fluorescence emission) and to form an image of the sample (e.g., a fluorescence image).

In some embodiments, the objective lens 210 may comprise a microscope objective such as an off-the-shelf objective. In some embodiments, the objective lens 210 may comprise a custom objective. An example of a custom objective lens and/or custom objective-tube lens combination is described below and in U.S. Pat. No. 11,060,138, which is incorporated herein by reference in its entirety. The objective lens 210 may be designed to reduce or minimize optical aberration at two locations such as two planes corresponding to two surfaces of a flow cell or other sample support structure. The objective lens 210 may be designed to reduce the optical aberration at the selected locations or planes, e.g., the first and second surfaces of a dual surface flow cell, relative to other locations or planes in the optical path. For example, the objective lens 210 may be designed to reduce the optical aberration at two depths or planes located at different distances from the objective lens as compared to the optical aberrations associated with other depths or planes at other distances from the objective. For example, in some embodiments, optical aberration may be less for imaging the first and second surfaces of a flow cell than that exhibited elsewhere in a region spanning from 1 to 10 mm from the front surface of the objective lens. Additionally, a custom objective lens 210 may in some embodiments be configured to compensate for optical aberration induced by transmission of fluorescence emission light through one or more portions of the sample support structure, such as a layer that includes one or more of the flow cell surfaces on which a sample is disposed, or a layer comprising a solution filling the fluid channel of a flow cell. These layers may comprise, for example, glass, quartz, plastic, or another transparent material having a refractive index, and which may introduce optical aberration.

In some embodiments, the objective lens 210 may have a numerical aperture (NA) of 0.6 or more. Such a numerical aperture may provide for reduced depth of focus and/or depth of field, improved background discrimination, and increased imaging resolution. In some embodiments, the objective lens 210 may have a numerical aperture (NA) of 0.6 or less. Such a numerical aperture may provide for increased depth of focus and/or depth of field. Such increased depth of focus and/or depth of field may increase the ability to image planes separated by a distance such as that that separates the first and second surfaces of a dual surface flow cell.

In some embodiments, the flow cell herein may include one or more surfaces and one or more substrates; at least one hydrophilic polymer coating layer; a plurality of oligonucleotide molecules attached to at least one hydrophilic polymer coating layer; and at least one discrete region of the one or more surfaces that comprises a plurality of clonally-amplified, sample nucleic acid molecules immobilized to the plurality of attached oligonucleotide molecules, wherein the plurality of immobilized clonally amplified sample nucleic acid molecules are present at a distance less than λ(2*NA), wherein λ is the center wavelength of an excitation energy source and NA is the numerical aperture of an imaging system.

In some embodiments, the objective lens 210 and/or the optical system 116 may be configured to provide a depth of field and/or depth of focus sufficiently large enough to image both the first and second interior surfaces of the flow cell or the bottom surface of the first substrate and the top surface of the bottom substrate of the test target. The depth of focus may enable imaging either sequentially by re-focusing the imaging module between imaging the first and second surfaces, or simultaneously by ensuring a sufficiently large depth of field and/or depth of focus, with comparable optical resolution. In some embodiments, the depth of field and/or depth of focus may be at least as large or larger than the distance separating the first and second surfaces of the flow cell to be imaged, such as the first and second interior surfaces of the flow cell. In some embodiments, the first and second surfaces, e.g., the first and second interior surfaces of a dual surface flow cell or a test target, may be separated, for example, by a distance ranging from about 10 μm to about 700 μm, or more. In some embodiments, the depth of field and/or depth of focus may thus range from about 10 μm to about 700 μm, or more.

In some designs, compensation optics (e.g., an “optical compensator” or “compensator”) may be moved into or out of an optical path in the imaging module, for example, an optical path by which light collected by the objective lens 210 is delivered to an image sensor, to enable the imaging module to image the first and second surfaces of the dual surface flow cell. The optical system 116 may be configured, for example, to image the first surface when the compensation optics are included in the optical path between the objective lens and an image sensor or photodetector array configured to capture an image of the first surface. In such a design, the imaging module may be configured to image the second surface when the compensation optics is removed from or not included in the optical path between the objective lens 210 and the image sensor or photodetector array configured to capture an image of the second surface. The need for an optical compensator may be more pronounced when using an objective lens 210 with a high numerical aperture (NA) value, e.g., for numerical aperture values of at least 0.6, least 0.65, at least 0.7, at least 0.75, at least 0.8, at least 0.85, at least 0.9, at least 0.95, at least 1.0, or higher. In some embodiments, the optical compensation optics (e.g., an optical compensator or compensator) comprise a refractive optical element such as a lens, a plate of optically-transparent material such as glass, or in the case of polarized light beams, a quarter-wave plate or half-wave plate, etc. Other configurations may be employed to enable the first and second surfaces to be imaged at different times. For example, one or more lenses or optical elements may be configured to be translated in and out of, or along, an optical path between the objective lens 210 and the image sensor.

In some embodiments, the optical system described herein allows imaging of the first and second surfaces without moving any compensation optics, e.g., a compensator, in, out, or along an optical path of the optical system herein. In some embodiments, the objective lens 210 is configured to provide sufficiently large depth of focus and/or depth of field to enable the first and second surfaces to be imaged with comparable optical resolution without such compensation optics moving into and out of an optical path in the imaging module, such as an optical path between the objective lens and the image sensor or photodetector array. Similarly, in some embodiments, the objective lens 210 is configured to provide sufficiently large depth of focus and/or depth of field to enable the first and second surfaces to be imaged with comparable optical resolution without optics being moved, such as one or more lenses or other optical components being translated along an optical path in the imaging module, such as an optical path between the objective lens and the image sensor or photodetector array. Examples of such objective lenses will be described in more detail below.

In some embodiments, the objective lens (or microscope objective) 210 may be configured to have reduced magnification. The objective lens 210 may be configured, for example, such that the fluorescence imaging module has a magnification of from less than 2× to less than 10× (as will be discussed in more detail below). Such reduced magnification may alter design constraints such that other design parameters can be achieved. For example, the objective lens 210 may also be configured such that the fluorescence imaging module has a large field-of-view (FOV) ranging, for example, from about 1.0 mm to about 5.0 mm (e.g., in diameter, width, length, or longest dimension) as will be discussed in more detail below.

In some embodiments, the objective lens 210 may be configured to provide the fluorescence imaging module with a field-of-view such that the FOV has diffraction-limited performance, e.g., less than 0.10, 0.12, or 0.15 waves of aberration over at least 60%, 70%, 80%, 90%, or 95% of the field.

In some embodiments, the objective lens 210 may be configured to provide the fluorescence imaging module with a field-of-view such that the FOV has diffraction-limited performance, e.g., a Strehl ratio of greater than 0.6, 0.7, or 0.8 over at least 60%, 70%, 80%, 90%, or 95% of the field.

Referring again to FIGS. 2A and 2B, the dichroic beam splitter or beam combiner 230 is disposed in the first optical path between the light source and the sample so as to illuminate the sample with one or more excitation beams. This dichroic beam splitter or combiner may also be in one or more second optical path(s) from the sample to the different optical channels used to detect the fluorescence emission. Accordingly, the dichroic filter 230 couples the first optical path of the excitation beam emitted by the illumination source 215 and second optical path of the emission light emitted by a sample specimen to the various optical channels where the light is directed to respective image sensors or photodetector arrays for capturing images of the sample.

In various embodiments, the dichroic filter 230, e.g., dichroic reflector or beam splitter or beam combiner, has a passband selected to transmit light from the illumination source 215 only within a specified wavelength band or possibly a plurality of wavelength bands that include the desired excitation wavelength or wavelengths. For example, the dichroic beam splitter 230 includes a reflective surface comprising a dichroic reflector that has a spectral transmissivity response that is, e.g., configured to transmit light having at least some of the wavelengths output by the light source that form part of the excitation beam. The spectral transmissivity response may be configured not to transmit (e.g., instead to reflect) light of one or more other wavelengths, for example, of one or more other fluorescence emission wavelengths. In some embodiments, the spectral transmissivity response may also be configured not to transmit (e.g., instead to reflect) light of one or more other wavelengths output by the light source.

Accordingly, the dichroic filter 230 may be utilized to select which wavelength or wavelengths of light output by the light source reach the sample. Conversely, the dichroic reflector in the dichroic beam splitter 230 has a spectral reflectivity response that reflects light having one or more wavelengths corresponding to the desired fluorescence emission from the sample and possibly reflects light having one or more wavelengths output from the light source that is not intended to reach the sample. Accordingly, in some embodiments, the dichroic reflector has a spectral transmissivity that includes one or more pass bands that transmit the light to be incident on the sample and one or more stop bands that reflect light outside the pass bands, for example, light at one or more emission wavelengths and possibly one or more wavelengths output by the light source that are not intended to reach the sample. Likewise, in some embodiments the dichroic reflector has a spectral reflectivity that includes one or more spectral regions configured to reflect one or more emission wavelengths and possibly one or more wavelengths output by the light source that are not intended to reach the sample and includes one or more regions that transmit light outside these reflection regions. The dichroic reflector included in the dichroic filter 230 may comprise a reflective filter such as an interference filter (e.g., a quarter-wave stack) configured to provide the appropriate spectral transmission and reflection distributions.

Although the optical system 116 shown in FIGS. 2A and 2B is configured such that the excitation beam is transmitted by the dichroic filter 230 to the objective lens 210, in some designs the illumination source 215 may be disposed with respect to the dichroic filter 230 and/or the dichroic filter 230 is configured (e.g., oriented) such that the excitation beam is reflected by the dichroic filter 230 to the objective lens 210. Similarly, in some such designs, the dichroic filter 230 is configured to transmit fluorescence emission from the sample and possibly transmit light having one or more wavelengths output from the light source that is not intended to reach the sample. A design where the fluorescence emission is transmitted instead of reflected may potentially reduce wavefront error in the detected emission and/or possibly have other advantages. In either case, in various embodiments the dichroic reflector 230 is disposed in the second optical path so as to receive fluorescence emission from the sample, at least some of which continues on to the detection channels 220.

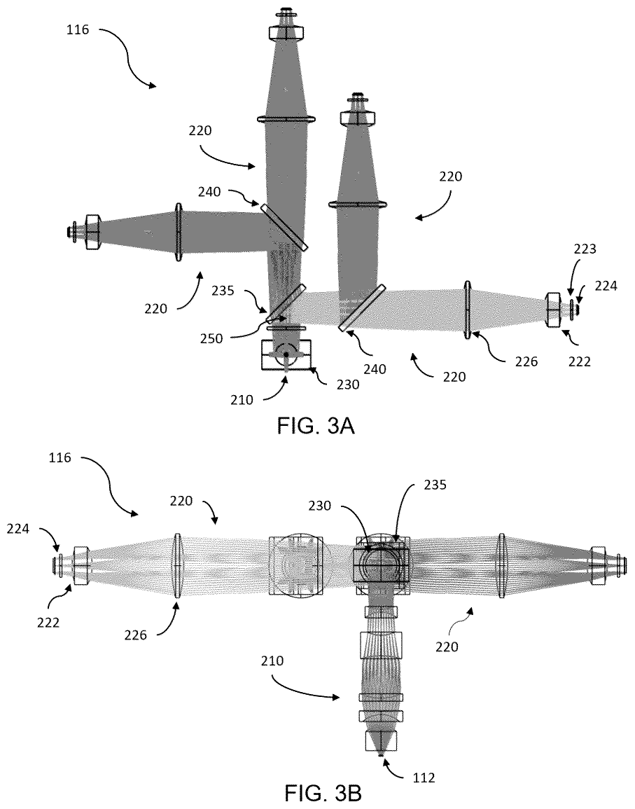

FIGS. 3A and 3B illustrate the optical paths within the optical system of FIGS. 2A and 2B. In the example shown in FIG. 2A and FIG. 3A, the detection channels 220 are disposed to receive fluorescence emission from a sample specimen that is transmitted by the objective lens 210 and reflected by the dichroic filter 230. In some embodiments, the detection channels 220 may be disposed to receive the portion of the emission light that is transmitted, rather than reflected, by the dichroic filter 230. In either case, the detection channels 220 may include optics or optical elements for receiving or reflecting at least a portion of the emission light.

In some embodiments, the detection channels 220 may include one or more lenses, such as tube lenses 221, and may include one or more image sensors or detectors 224 such as photodetector arrays (e.g., CCD or CMOS sensor arrays) for imaging or otherwise producing a signal based on the received light. The tube lenses may, for example, comprise one or more lens elements configured to form an image of the sample onto the sensor or photodetector array to capture an image thereof. Additional discussion of detection channels is included in U.S. Pat. No. 11,060,138, which is incorporated herein by reference in its entirety. In some embodiments, improved optical resolution may be achieved using an image sensor having relatively high sensitivity, small pixels, and high pixel count, in conjunction with a suitable sampling scheme, which may include oversampling or undersampling. In some embodiments, the detection channels 220 may include an emission filter 223 that can be positioned between the image sensor 224 and the tube lens 221. The emission filter 223 can be optional. The emission filter can be a bandpass filter that functions to remove certain wavelengths before the signals are captured by the image sensor. In some embodiments, the detection channel 220 may include one or more corresponding dichroic filters that may correspond to a single detection channel or may be shared by two or more detection channels. The dichroic filter 230, 235, 240 may comprise one or more of: a dichroic mirror, a dichroic reflector, a dichroic beam splitter, or a dichroic beam combiner. In some embodiments, the dichroic filter 230, 235, 240 may be configured to transmit light in a first spectral region (or wavelength range) and reflect light having a second spectral region (or wavelength range). The first spectral region may include one or more spectral bands, e.g., one or more spectral bands in the ultraviolet and blue wavelength ranges. The second spectral region may include one or more spectral bands, e.g., one or more spectral bands extending from the green to red and infrared wavelengths. In other embodiments, the first spectral region may include one or more spectral bands, e.g., one or more spectral bands extending from the green to red and infrared wavelengths. The second spectral region may include one or more spectral bands, e.g., one or more spectral bands in the ultraviolet and blue wavelength ranges. Other spectral regions or wavelength ranges are also possible.

The systems and methods herein may be used to optically autofocus one or more of the detection channels so that the focal planes of the objective lens substantially align with the sample to be imaged, and the sample can be in-focus in the acquired flow cell images. In embodiments where each detection channel may include its corresponding objective lens (not shown) and tube lens, the focal plane may be of the corresponding objective lens in that detection channel.

FIGS. 3A and 3B are ray tracing diagrams illustrating optical paths of the optical system 116 of FIGS. 2A and 2B. FIG. 3A corresponds to a top view of the optical system. FIG. 3B corresponds to a side view of the optical system. The optical system 116 illustrated in these figures includes four detection channels 220. However, it will be understood that the optical system may equally be implemented in systems including more or fewer than four detection channels 220. For example, the multi-channel systems disclosed herein may be implemented with as few as one detection channel 220, or as many as two detection channels 220, three detection channels 220, four detection channels 220, five detection channels 220, six detection channels 220, seven detection channels 220, eight detection channels 220, or more than eight detection channels 220, without departing from the scope of the present disclosure.

The non-limiting example of optical system 116 illustrated in FIGS. 3A and 3B includes four detection channels 220, a dichroic filter 230 that reflects a beam 250 of emission light, a second dichroic filter (e.g., a dichroic beam splitter) 235 that splits the beam 250 into a transmitted portion and a reflected portion, and two channel-specific dichroic filters (e.g., dichroic beam splitters) 240 that further split the transmitted and reflected portions of the beam 250 among individual detection channels 220. The dichroic reflecting surface in the dichroic beam splitters 235 and 240 for splitting the beam 250 among detection channels are shown disposed at 45 degrees relative to a central beam axis of the beam 250 or an optical axis of the imaging module. However, as discussed below, an angle smaller than 45 degrees may be employed and may offer advantages such as sharper transitions from pass band to stop band.

The different detection channels 220 may each include an image sensor 224 such as a photodetector array (e.g., a CCD or CMOS detector array). The different detection channels 220 may further include optics 226 such as lenses (e.g., one or more tube lenses each comprising one or more lens elements) disposed to focus the portion of the emission light entering the detection channel 220 at a focal plane coincident with a plane of the photodetector array 224. The optics 226 (e.g., a tube lens) combined with the objective lens 210 are configured to form an image of the sample onto the image sensor 224, e.g., photodetector array, to capture an image of the sample, for example, an image of a surface on the flow cell or other sample support structure after the sample has bound to that surface. Accordingly, such an image of the sample or the test target may comprise a plurality of fluorescent emitting spots or regions across a spatial extent of the sample support structure where the sample is emitting fluorescence light. The objective lens 210 together with the optics 226 (e.g., tube lens) may provide a field-of-view (FOV) that includes a portion of the sample or the entire sample. Similarly, the photodetector array 224 of the different detection channels 220 may be configured to capture images of a full field-of-view (FOV) provided by the objective lens and the tube lens, or a portion thereof. In some embodiments, the photodetector array 224 of some or all detection channels 220 can detect the emission light emitted by a sample disposed on the sample support structure, e.g., a surface of the flow cell or a portion thereof, and record electronic data representing an image thereof. In some embodiments, the photodetector array 224 of some or all detection channels 220 can detect features in the emission light emitted by a specimen without capturing and/or storing an image of the sample disposed on the flow cell surface and/or of the full field-of-view (FOV) provided by the objective lens and optics 226 and/or 222 (e.g., elements of a tube lens). In some embodiments, the FOV of the disclosed imaging modules (e.g., that provided by the combination of objective lens 210 and optics 226 and/or 222) may range, for example, between about 1 mm and 5 mm (e.g., in diameter, width, length, or longest dimension) as will be discussed below. The FOV may be selected, for example, to provide a balance between magnification and resolution of the imaging module and/or based on one or more characteristics of the image sensors and/or objective lenses. For example, a relatively smaller FOV may be provided in conjunction with a smaller and faster image sensor to achieve high throughput.

In some embodiments, one or more image sensors of the optical system may be used for both imaging and autofocusing. The one or more image sensors may be used to acquire one or more images for determining the z shift for autofocusing. In some embodiments, the image obtained by image sensor for AF purposes comprises a single image. The single image may comprises a field of view (FOV) that is identical to a size of the image sensor or the AF sensor along the x or y axis. FIG. 6B shows an example single image 600 with a size along x axis that is identical to the size of the image sensor along the x axis. In some embodiments, the image(s) comprise a length (along the x axis) or width (along the y axis) that is in a range from 0.1 mm to 5 cm. In some embodiments, the image(s) comprise a length (along the x axis) or width (along the y axis) that is in a range from 0.5 mm to 9 mm. In some embodiments, the image(s) comprise a length (along the x axis) or width (along the y axis) that is in a range from 0.8 mm to 4 mm. In some embodiments, the image comprises the FOV that is identical to a size of the image sensor along the x axis when the tilt angle is about the x axis. In some embodiments, the image comprises the FOV that is identical to a size of the image sensor along the y axis when the tilt angle is about the y axis.

FIG. 6A shows the sample stage and its tilting angle relative the focal plane of the objective or otherwise of the optical system. The tilting angle in this embodiment is along the x-axis. In some embodiments, the tilting angle is tilting about the y axis and within the x-z plane. In some embodiments, the image is acquired of the sample or test target while the sample stage remains tilted. In some embodiments, the image comprises a fluorescent or otherwise optical signal emitted from the sample or the test target disclosed herein. In this particular embodiment, the size of the image along x is identical to the image sensor size along the x axis. In this embodiment, the size of the image along y is also identical to the image sensor size along the y axis. In other embodiments, the size of the image along the y and/or x axes may be reduced to save image processing time and storage space.

Although tilting is shown as tilting the sample stage in FIG. 6A, any other tilting schemes may be used to achieve the same effect of having a predetermined tilting angle from the image sensor relative to the sample stage. For example, in some embodiments, the image sensor(s) is connected to a motor, e.g., a hexapod, so that the image sensor may be tilted by the tilt angle controlled automatically by the motor, while the sample may remain still. In some embodiments, to achieve the tilting effect in the image, one of the image sensor and the sample stage may remain still and the other one may be tilted. In some embodiments, to achieve the tilting effect in the image, one of the image sensor and the sample stage may both be tilted, but each with a smaller angle to achieve the total tilting effect of the sum of the two smaller angles.

In some embodiments, tilting and then de-tilting the image sensor(s) may not be preferred because such motion may affect optical alignment and other features of the optical system. In some embodiments, the image sensor(s) remain immobilized to the baseplate during autofocusing, but other optical elements, e.g., the sample stage, can be tilted to achieve similar image(s) that can be used for determining z shift as if the image sensor were tilted.

Referring again to FIGS. 3A and 3B, in some embodiments, the optics 226 in the detection channel (e.g., the tube lens 221) may be configured to reduce optical aberration in images acquired using the optics 226 in combination with the objective lens 210. In some embodiments the imaging module 200 may comprise multiple detection channels for imaging at different emission wavelengths, and the optics 226 (e.g., the tube lens) for different detection channels may have different designs to reduce aberration for the respective emission wavelengths at which that particular channel is configured to image. In some embodiments, the optics 226 (e.g., the tube lens) may be configured to reduce aberrations when imaging a specific surface (e.g., a plane, object plane, etc.) on the sample support structure comprising fluorescing sample sites disposed thereon as compared to other locations (e.g., other planes in object space). In some embodiments, the optics 226 (e.g., the tube lens) may be configured to reduce aberrations when imaging the first and second surfaces (e.g., first and second planes, first and second object planes, etc.) on a dual surface sample support structure (e.g., a dual surface flow cell or test target) having fluorescing sample sites disposed thereon as compared to other locations (e.g., other planes in object space). For example, the optics 226 in the detection channel (e.g., the tube lens) may be designed to reduce the aberration at two depths or planes located at different distances from the objective lens as compared to the aberrations associated with other depths or planes at other distances from the objective. For example, optical aberration may be less for imaging the first and second surfaces than elsewhere in a region from about 1 to about 10 mm from the objective lens. Additionally, custom optics 226 in the detection channel (e.g., a tube lens) may in some embodiments be configured to compensate for aberration induced by transmission of emission light through one or more portions of the sample support structure such as a layer that includes one of the surfaces on which the sample is disposed as well as possibly a solution adjacent to and in contact with the surface on which the sample is disposed. The layer comprising one of the surfaces on which the sample is disposed may comprise, e.g., glass, quartz, plastic, or another transparent material having a refractive index, and which introduces optical aberration. Custom optics 226 in the detection channel (e.g., the tube lens), for example, may in some embodiments be configured to compensate for optical aberration induced by a sample support structure, e.g., a coverslip or flow cell wall, or other sample support structure components, as well as possibly a solution adjacent to and in contact with the surface on which the sample is disposed.

In some embodiments, the optics 226 in the detection channel 220 (e.g., a tube lens) are configured to have reduced magnification. The optics 226 in the detection channel (e.g., a tube lens) may be configured, for example, such that the fluorescence imaging module has a magnification of less than, for example, 10×, as will be discussed further below. Such reduced magnification may alter design constraints such that other design parameters can be achieved. For example, the optics 226 (e.g., a tube lens) may also be configured such that the fluorescence imaging module has a large field-of-view (FOV), for example, of at least 1.0 mm or larger (e.g., in diameter, width, length, or longest dimension), as will be discussed further below.

In some embodiments, the optics 226 (e.g., a tube lens) may be configured to provide the fluorescence imaging module with a field-of-view as indicated above such that the FOV has less than 0.15 waves of aberration over at least 60%, 70%, 80%, 90%, or 95% of the field, as will be discussed further below.

Referring again to FIGS. 3A and 3B, in various embodiments, a sample immobilized on the flow cell or a test target is located at or near a focal position 212 of the objective lens 210. In embodiments where the optical system does not have an objective, the focal position 212 can be of the whole optical system. Details of the optical system without any objective lens are disclosed in PCT application No. PCT/US2024/012802, which is hereby incorporated by reference in its entirety. As described with reference to FIGS. 2A and 2B, a light source such as a laser source provides an excitation beam to the sample to induce fluorescence. At least a portion of a fluorescence emission is collected by the objective lens 210 as emission light. The objective lens 210 may transmit the emission light toward the dichroic filter 230, which reflects some or all of the emission light as the beam 250 incident upon the second dichroic filter 235 and to the different detection channels, each comprising optics 226 that form an image of the sample (e.g., a plurality of fluorescing sample sites on a surface of a sample support structure) onto a corresponding image sensor 224 in the detection channels, e.g., a photodetector array.

As discussed above, in some embodiments, the sample support structure comprises a flow cell or testing target having two surfaces (e.g., two interior surfaces, a first surface and a second surface, etc.) containing sample sites that emit fluorescent emission. These two surfaces may be separated by a distance from each other in the longitudinal (Z) direction along the direction of the central axis of the excitation beam and/or the optical axis of the objective lens. This separation may correspond, for example, to a flow channel within the flow cell. Analytes or reagents may be flowed through the flow channel and contact the first and second interior surfaces of the flow cell, which may thereby be contacted with a binding composition such that fluorescence emission is radiated from a plurality of sites on the first and second interior surfaces. The imaging optics (e.g., objective lens 210) may be positioned at a suitable distance (e.g., a distance corresponding to the working distance) from the sample to form in-focus images of the sample on one or more image sensors or detector arrays 224. In various designs, the objective lens 210 (possibly in combination with the optics 226) may have a depth of field and/or depth of focus that is at least as large as the longitudinal separation between the first and second surfaces. The objective lens 210 and the optics 226 (of each detection channel) can thus simultaneously form images of both the first and the second flow cell surfaces on the photodetector array 224, and these images of the first and second surfaces are both in focus and have comparable optical resolution (or may be brought into focus with only minor refocusing of the objects to acquire images of the first and second surfaces that have comparable optical resolution). In various embodiments, compensation optics need not be moved into or out of an optical path of the imaging module (e.g., into or out of the first and/or second optical paths) to form in-focus images of the first and second surfaces that are of comparable optical resolution. Similarly, in various embodiments, one or more optical elements (e.g., lens elements) in the imaging module (e.g., the objective lens 210 or optics 226) need not be moved, for example, in the longitudinal direction along the first and/or second optical paths to form in-focus images of the first surface in comparison to the location of said one or more optical elements when used to form in-focus images of the second surface. In some embodiments, the imaging module includes an autofocus system configured to quickly and sequentially refocus the imaging module on the first and/or second surface such that the images have comparable optical resolution. In some embodiments, an objective lens 210 and/or optics 226 are configured such that both the first and second flow cell surfaces are in focus simultaneously with comparable optical resolution without moving an optical compensator into or out of the first and/or second optical path, and without moving one or more lens elements (e.g., objective lens 210 and/or optics 226 (such as a tube lens)) longitudinally along the first and/or second optics path. In some embodiments, images of the first and/or second surfaces, acquired either sequentially (e.g., with refocusing between surfaces) or simultaneously (e.g., without refocusing between surfaces) using the novel objective lens and/or tube lens designs disclosed herein, may be further processed using a suitable image processing algorithm to enhance the effective optical resolution of the images such that the images of the first and second surfaces have comparable optical resolution. In various embodiments, the sample plane is sufficiently in focus to resolve sample sites on the first and/or second flow cell surfaces, the sample sites being closely spaced in lateral directions (e.g., in the X and Y directions).

As discussed herein, the dichroic filters may comprise interference filters that selectively transmit and reflect light of different wavelengths based on the principle of thin-film interference, using layers of optical coatings having different refractive indices and particular thickness. Accordingly, the spectral response (e.g., transmission and/or reflection spectra) of the dichroic filters implemented within multi-channel fluorescence imaging modules may be at least partially dependent upon the angle of incidence, or range of angles of incidence, at which the light of the excitation and/or emission beams are incident upon the dichroic filters. Such effects may be especially significant with respect to the dichroic filters of the detection optical path (e.g., the dichroic filters 235 and 240 of FIGS. 3A and 3B).

### Systems and Methods for Autofocusing

In one aspect, the disclosure provides a method for focusing an optical system, the method comprising: (a) receiving an image of a substrate of the optical system, wherein a portion and less than all of the image is in focus, and wherein the portion of the image in focus is offset from a center of the image; (b) determining, using at least a distance from the portion of the image in focus and the center of the image, an amount of defocus in the image; and (c) adjusting a parameter of the optical system to adjust for the defocus. In some embodiments, the image may be an image of a flow cell. In some embodiments, the image may be acquired using an image sensor, wherein light collected by the objective lens 210 may be delivered to the image sensor. In some cases, the flow cell 112 may be configured to capture DNA fragments and form DNA sequences for base-calling. In some cases, the defocus can be a z-shift as described elsewhere herein. The defocus may be the distance from the imaging plane to a focal plane of the optical system.

In some cases, adjusting a parameter of the optical system to adjust for the defocus may comprise moving the sample stage relative to the focal plane of an objective lens of the optical system by the determined amount of defocus, thereby autofocusing the optical system. In some embodiments, the adjusting may be automated adjusting. For example, the sample stage may be motorized or otherwise connected to a motor such that the movement of the sample stage may be automatic in response to receiving an instruction provided by a user or a computer system as disclosed herein.

In some embodiments, the image may be received from an autofocus element. Such autofocus elements may include, but are not limited to, one or more of: an autofocus illumination sources, an autofocus sensor 202, an autofocus tube lens, and a dichroic filter or beam splitter.

In some embodiments, determining the amount of defocus in the image using the methods disclosed herein may be done in at most 600 ms. In some embodiments, determining the amount of defocus may be done in at most 500 ms. In some embodiments, determining the amount of defocus may be done in at most 400 ms. In some embodiments, determining the amount of defocus may be done in at most 300 ms. In some embodiments, determining the amount of defocus may be done in at most 200 ms. In some embodiments, determining the amount of defocus may be done in at most 100 ms.

In some embodiments, the method further comprises imaging a substrate, wherein imaging a substrate comprises using a light source and a detector to generate an image. Any suitable light source configured to produce light of a predetermined excitation wavelength or wavelengths may be used. The substrate may be as described elsewhere herein (e.g., a flow cell, a glass substrate, etc.).

In some embodiments, the determining of the amount of defocus may be performed using only a single image and not multiple images. Using only a single image reduces time consumption and computational complexity when compared with existing autofocusing methods that rely on at least two images and/or machine learning algorithms.

In some embodiments, the image may comprise a length or width that is in a range from about 0.1 millimeters to about 5 centimeters. In some embodiments, the image may comprise a length or a width that is in a range from about 0.5 millimeters to about 9 millimeters.

In some embodiments, the error in the amount of defocus from the true amount of defocus may be at most about 400 nanometers. In some embodiments, the error in the amount of defocus from the true amount of defocus may be at most about 350 nanometers. In some embodiments, the error in the amount of defocus from the true amount of defocus may be at most about 300 nanometers. In some embodiments, the error in the amount of defocus from the true amount of defocus may be at most about 250 nanometers. In some embodiments, the error in the amount of defocus from the true amount of defocus may be at most about 200 nanometers. In some embodiments, the error in the amount of defocus from the true amount of defocus may be at most about 150 nanometers. In some embodiments, the error in the amount of defocus from the true amount of defocus may be at most about 100 nanometers. In some embodiments, the error in the amount of defocus from the true amount of defocus may be at most about 50 nanometers.

In some embodiments, the center of the in focus region may be determined using an image processing algorithm. In some embodiments, the image processing algorithm may comprise determining the center of the in focus region by separating the image into a predetermined number of regions and using a sum or average intensity of each region to identify the location of the in focus region. In some embodiments, the sum or average image intensity of each region can be used to identify the approximate location of the in-focus region because the in-focus region of the image may have a higher intensity than the out-of-focus dark regions. In some embodiments, the image intensity (e.g., intensity projection) and/or the spatial frequency (e.g. a Fourier transform of the intensities) may be used to locate the center of the in-focus region 606a. In some embodiments, information about the geometrical patterns in the image may be used to the determine which image processing algorithm or algorithms will be used to find the center 606a.

In one aspect, the present disclosure provides a method of focusing an optical system, the method comprising: (a) imaging, using a detector, a substrate tilted at a tilt angle, wherein an image of said substrate comprises an in focus portion and an out-of-focus portion; (b) determining, using a processor, a defocus of the optical system based at least in part on said tilt angle and a distance of said in focus portion from a center of said image; (c) adjusting said substrate to remove said tilt angle; and (d) adjusting said substrate by said defocus, thereby focusing said optical system.

In some embodiments, determining the defocus of the optical system further comprises defining a vector of the in-focus portion to the center of the image. In some cases, the vector may be the x-y plane shift as defined elsewhere herein.

The method of focusing the optical system may further comprise a motor coupled to the substrate, wherein the motor is configured to impart the tilt angle. In some cases, the sample stage may be motorized or otherwise connected to a motor such that movement of the sample stage can be automatic in response to receiving an instruction either provided by a user or by a computer system disclosed herein.

In some embodiments, the image may be received from an autofocus element. Such autofocus elements may include, but are not limited to, one or more of: an autofocus illumination sources, an autofocus sensor 202, an autofocus tube lens, and a dichroic filter or beam splitter. In some embodiments, the optical system may include an image sensor 224 such as a photodetector array (e.g., a CCD or CMOS detector array).

In some embodiments, the method may further comprise tilting the substrate to the tilt angle. In some cases, the method may include an operation of tilting a sample stage of the optical system by the tilt angle, wherein the sample is immobilized on the sample stage. The tilting of the sample stage may be relative to the focal plane of objective. The tilting may alternatively be relative to the focal plane of the optical system when the optical system lacks any objective lens. In some alternative embodiments, the operation may include tilting an image sensor or a dedicated autofocusing sensor instead of the sample stage by the tilt angle to achieve equivalent effect on the image to be acquired. Such tilting is relative to the focal plane of the objective lens or the focal plane of the optical system. However, tilting the sample stage may be preferred since tilting the image sensor in each autofocusing process and de-tilting it back for imaging after autofocusing is completed may introduce inconsistency or errors in the optical alignment of the image sensor to the other optical elements of the optical system such as the corresponding tube lens. Tilting the autofocusing sensor has the advantage that it does not need to be de-tilted back. Tilting the autofocusing sensor may also have the advantage that the autofocusing sensor may remain tilted since it is dedicated for autofocusing usage only. However, tilting the autofocusing sensor may add additional cost and complexity to the optical system compared to those optical systems without autofocusing sensors.

In some embodiments, the method may include an operation of de-tilting the substrate. The method may include de-tilting the tilted sample stage, the tilted image sensor, or the tilted autofocusing sensor that is tilted in operation 510 back by the tilt angle (in an opposite direction) to return it to the spatial position before the tilting operation 510.

In some embodiments, the tilting of the substrate may be a tilting of a plane orthogonal to an optical axis of the optical system. In some embodiments, the vector as defined herein may be a distance from the image center that corresponds to an intersecting point of the optical axis and the image plane (e.g., x-y plane) to a center of the in-focus region. The center of the in-focus region can be on a straight line that is within the x-y plane. The center of the in-focus region can be on a straight line that that is orthogonal to the tilting axis of the tilt angle.

In some embodiments, the tilt angle may be from about 0.01 to about 89 degrees. In some embodiments, the tilt angle may be from about 0.05 to about 15 degrees. In some embodiments, the tilt angle may be from about 0.05 degrees to about 8 degrees.

In some embodiments, the angular resolution of the tilt angle may be from about 0.001 degrees to about 0.2 degrees. In some embodiments, the angular resolution of the tilt angle may be from about 0.01 degrees to about 0.1 degrees. In some embodiments, the angular resolution of the tilt angle may be from about 0.01 degrees to 0.08 degrees.

In some embodiments, the determining of the defocus in this method may be performed using only the single image and not multiple images. Using only a single image reduces time consumption and computational complexity when compared with existing autofocusing methods that rely on at least two images and/or machine learning algorithms.

In some embodiments, the substrate may comprise a flow cell, the flow cell comprising: one or more surfaces; at least one hydrophilic polymer coating layer; a plurality of oligonucleotide molecules attached to the at least one hydrophilic polymer coating layer; and at least one discrete region of the one or more surfaces that comprise a plurality of clonally-amplified nucleic acid molecules immobilized to the plurality of attached oligonucleotide molecules, wherein the plurality of immobilized clonally amplified sample nucleic acid molecules is present at a distance less than λ(2*NA), wherein λ is the center wavelength of an excitation energy source and NA is the numerical aperture of said optical system.

In some cases, the hydrophilic polymer coating may comprise hydrophilic polymers that are non-specifically adsorbed or covalently grafted to the support. Passivation may be performed utilizing poly(ethylene glycol) (PEG, also known as polyethylene oxide (PEO) or polyoxyethylene) or other hydrophilic polymers with different molecular weights and end groups that are linked to a support using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface.

In some embodiments, the substrate comprises a beaded flow cell. In some embodiments, the beaded flow cell comprises a surface comprising fluorescent beads chemically immobilized to the substrate. In some embodiments, the fluorescent beads are randomly distributed on the surface. In some embodiments, the fluorescent beads are patterned on the surface. In some embodiments, the fluorescent beads comprise at least about 4 types of beads configured to emit different colors in response to excitation from a laser. In some embodiments, the fluorescent beads can be microbeads that are commercially available. In some embodiments, the microbeads are customized. In some embodiments, the beaded flow cell comprises a surface coated with fluorescent beads that are chemically immobilized to the surface. The fluorescent beads may comprise one, two, three, four, five or six different types of beads that emits different colors and/or light at different frequencies in response to an optical excitement, e.g., a laser light. The fluorescent beads may emit fluorescent light of one or more wavelengths in response to laser excitement.

In some embodiments, an error in the distance from the focal plane to a true distance from the focal plane may be at most about 400 nanometers. In some embodiments, an error in the distance from the focal plane to a true distance from the focal plane may be at most about 350 nanometers. In some embodiments, an error in the distance from the focal plane to a true distance from the focal plane may be at most about 300 nanometers. In some embodiments, an error in the distance from the focal plane to a true distance from the focal plane may be at most about 250 nanometers. In some embodiments, an error in the distance from the focal plane to a true distance from the focal plane may be at most about 200 nanometers. In some embodiments, an error in the distance from the focal plane to a true distance from the focal plane may be at most about 150 nanometers. In some embodiments, an error in the distance from the focal plane to a true distance from the focal plane may be at most about 100 nanometers. In some embodiments, an error in the distance from the focal plane to a true distance from the focal plane may be at most about 50 nanometers.

In some embodiments, following the adjusting of the substrate by the defocus, thereby focusing the optical system, the optical system may be used for imaging a nucleic acid molecule immobilized to the substrate in a first flow cycle. In some embodiments, the operations of imaging the substrate tilted at the tilt angle, determining the defocus, adjusting the substrate to remove the tilt angle, and adjusting the substrate by the defocus to focus the optical system may be repeated for a second flow cycle.

In one aspect, the present disclosure provides a method of focusing an optical system, the method comprising: (a) imaging, using a detector tilted at a tilt angel, a substrate, wherein an image of the substrate comprises an in focus portion and an out-of-focus portion; (b) determining, using a processor, a defocus of the optical system based at least in part on the tilt angle and a distance of the in focus portion from a center of the image; and (c) adjusting the substrate by the defocus, thereby focusing the optical system.

In some embodiments, the method further comprises adjusting the substrate by the defocus, thereby placing the substrate into focus. In some embodiments, the method further comprises tilting the detector to the tilt angle. In some cases, the method of tilting the detector may include tilting an image sensor or a dedicated autofocusing sensor instead of the sample stage by the tilt angle. In some embodiments, the method further comprises, subsequent to adjusting the substrate by the defocus, de-tilting the detector. In some embodiments, the tilting is the tilting of a plane orthogonal to an optical axis of the optical system.

In some embodiments, the tilt angle may be from about 0.01 to about 89 degrees. In some embodiments, the tilt angle may be from about 0.05 to about 15 degrees. In some embodiments, the tilt angle may be from about 0.05 degrees to about 8 degrees.

In some embodiments, the determining of the defocus in this method may be performed using only the single image and not multiple images. Using only a single image reduces time consumption and computational complexity when compared with existing autofocusing methods that rely on at least two images and/or machine learning algorithms.

In some embodiments, an error in said amount of defocus from a true amount of defocus is at most about 400 nanometers. In some embodiments, an error in said amount of defocus from a true amount of defocus is at most about 350 nanometers. In some embodiments, an error in said amount of defocus from a true amount of defocus is at most about 300 nanometers. In some embodiments, an error in said amount of defocus from a true amount of defocus is at most about 250 nanometers. In some embodiments, an error in said amount of defocus from a true amount of defocus is at most about 200 nanometers. In some embodiments, an error in said amount of defocus from a true amount of defocus is at most about 150 nanometers. In some embodiments, an error in said amount of defocus from a true amount of defocus is at most about 100 nanometers. In some embodiments, an error in said amount of defocus from a true amount of defocus is at most about 50 nanometers.

In some embodiments, the method further comprises calibrating a pivot point of the optical system. In some embodiments, calibrating the pivot point comprises de-tilting the substrate, the detector, or an autofocus sensor. In some cases, calibrating the pivot point can include an operation of determining a pivot point offset based on a regional center of an in-focus region of a calibration image and an image center of the calibration image. The pivot point offset can be from the optical axis 1099. Referring to FIG. 9, the pivot point offset is the distance between the center of the image 1099 that corresponds to the optical axis intersection point with the x-y plane or the image plane and the regional center 1091 of the in-focus region. Without pivot point offset, the center of the image 1099 may overlap with the center of the in-focus region 1091. With the pivot point offset, the center of the in-focus region may shift away from the center of the image after tilting. The regional center can be determined using various image processing algorithms that can be used in operation 530. After the pivot point offset is determined, the calibration operation can include de-tilting the sample stage, the image sensor, or the autofocusing sensor back into the position before calibration operation starts.

In one aspect, the present disclosure provides an optical system, the optical system comprising: a substrate; an autofocus module configured to take an image of the substrate, wherein the image comprises an in focus portion and an out-of-focus portion, and wherein the substrate or the autofocus module is tilted at a tilt angle; and a processor configured to determine a defocus of the substrate to a focal plane of the optical system using at least a distance from the in focus portion to a center of the image and the tilt angle.

In some embodiments, the processor may include one or more of: a processing unit, an integrated circuit, or their combinations. For example, the processing unit may include a central processing unit (CPU) and/or a graphic processing unit (GPU). The integrated circuit may include a chip such as a field-programmable gate array (FPGA).

In some embodiments, the substrate may be tilted at a tilt angle. In some embodiments, the processor may use the tilt angle of the substrate in determining the defocus. In some embodiments, the autofocus module may be tilted at a tilt angle. In some embodiments, the processor may use the tilt angle of the autofocus module in determining the defocus.

In some embodiments, the autofocus module comprises an illumination source and a detector. In some embodiments, the illumination source is configured to illuminate at least a portion of the substrate, and the detector is configured to image at least a portion of the portion of the substrate.

In some embodiments, the tilt angle may be from about 0.01 to about 89 degrees. In some embodiments, the tilt angle may be from about 0.05 to about 15 degrees. In some embodiments, the tilt angle may be from about 0.05 degrees to about 8 degrees.

In some embodiments, the determining of the defocus in this system may be performed using only the single image and not multiple images. Using only a single image reduces time consumption and computational complexity when compared with existing autofocusing methods that rely on at least two images and/or machine learning algorithms.

In some embodiments, an error in said amount of defocus from a true amount of defocus is at most about 400 nanometers. In some embodiments, an error in said amount of defocus from a true amount of defocus is at most about 350 nanometers. In some embodiments, an error in said amount of defocus from a true amount of defocus is at most about 300 nanometers. In some embodiments, an error in said amount of defocus from a true amount of defocus is at most about 250 nanometers. In some embodiments, an error in said amount of defocus from a true amount of defocus is at most about 200 nanometers. In some embodiments, an error in said amount of defocus from a true amount of defocus is at most about 150 nanometers. In some embodiments, an error in said amount of defocus from a true amount of defocus is at most about 100 nanometers. In some embodiments, an error in said amount of defocus from a true amount of defocus is at most about 50 nanometers.

In some embodiments, the autofocus module may comprise one or more of: an autofocus illumination source, an autofocus sensor, an autofocus tube lens, a dichroic filter, or a beam splitter.

In some embodiments, the optical system may comprise one or more image sensors. In some embodiments, the one or more image sensors may be used for both imaging the substrate and focusing the optical system. In some embodiments, the image is acquired by the autofocus module, wherein said autofocus module is only configured for autofocusing and not for imaging the substrate after autofocusing is completed.

In some embodiments, methods disclosed herein enable accurate and reliable autofocusing of the optical system 116 before imaging, e.g., imaging of sequencing reactions of a sample immobilized on a flow cell. Refocusing of the optical system can occur whenever it is needed. For example, refocusing of the optical system can occur before imaging of a different sample, a different surface of the same flow cell, a different tile or subtile of the flow cell, and/or the same spatial region of the sample but in a different flow cycle.

In some embodiments, the methods can be utilized for focusing the optical system before imaging the sample nucleic acid molecules immobilized to the flow cell in a first flow cycle and then refocusing before imaging the sample molecules again in a second flow cycle in the sequencing run. In some embodiments, the methods can be utilized for focusing the optical system before imaging the sample nucleic acid molecules immobilized on a first surface in a first flow cycle in a sequencing run, and for refocusing the optical system before imaging the sample nucleic acid molecules immobilized to the first surface or to a second surface in a second flow cycle in the sequencing run. In some embodiments, the methods can be utilized for focusing the optical system before imaging the sample nucleic acid molecules immobilized to a first surface in a first flow cycle in a sequencing run, and for refocusing the optical system for imaging the sample nucleic acid molecules immobilized to a second surface in the first flow cycle or a second flow cycle in the sequencing run.

In some embodiments, the methods can be utilized for focusing the optical system for imaging samples with various signal to noise ratios (SNR) or contrast to noise ratios (CNR). For example, the methods can be used to focus the optical system using samples (or to image samples) with a traditional CNR that can be achieved using conventional supports and hybridization, amplification, and/or NGS sequencing protocols. For example, the methods can be used to focus the optical system for imaging samples with any CNR that is higher than a traditional CNR which can be achieved using conventional supports and hybridization, amplification, and/or NGS sequencing protocols.

In some embodiments, the methods can be utilized for focusing the optical system for imaging low or unbalanced nucleotide diversity sequencing data. In some embodiments, the methods can be utilized for focusing the optical system with samples that are of unbalanced nucleotide diversity sequencing data. The nucleotide diversity of a population of immobilized nucleotide acid molecules can refer to the relative proportion of nucleotides A, G, C and T that are present in each sequencing cycle. A balanced diversity data can generally have approximately equal proportions of all four nucleotides represented in each cycle of a sequencing run. An unbalanced diversity data can generally include a high proportion of certain nucleotides and low proportion of other nucleotides, e.g., A, G, C, and T are of 5%, 30%, 25%, and 40%, respectively of the total nucleotides, in one or multiple cycles.

In some embodiments, the methods can be utilized for focusing the optical system for imaging samples that include nucleotide acid molecules that has been amplified using various methods into template modules, e.g., via rolling circle amplification. In some embodiments, the methods can be utilized for focusing the optical system for imaging samples with various signal densities. In some embodiments, the density of template nucleotide molecules immobilized to the support is about 10²-10¹⁵ per mm².

In some embodiments, the methods can be utilized for focusing the optical system using samples that include nucleotide acid molecules that has been amplified using various methods into template modules, e.g., via rolling circle amplification. In some embodiments, the methods can be utilized for focusing the optical system using samples with various signal densities. In some embodiments, the density of template nucleotide molecules immobilized to the support is about 10²-10¹⁵ per mm².

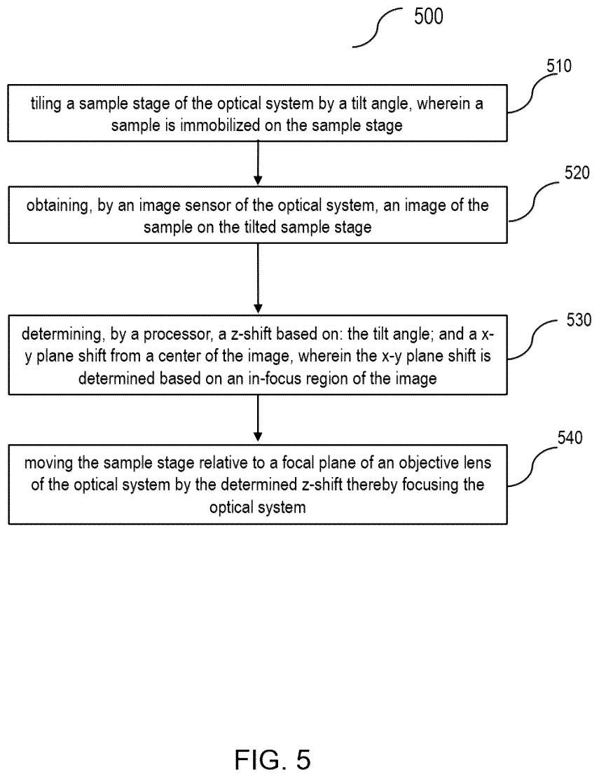

FIG. 5A shows a flow chart of a method for performing autofocusing using a single image acquired of a tilted sample, according to some embodiments. The methods 500 may include some or all of the operations disclosed herein. The operations may be performed in the order that is described herein, but is not limited to the order that has been described herein.

Some or all operations of the methods 500 may be performed by one or more processors disclosed herein. In some aspects, the processor may include one or more of: a processing unit, an integrated circuit, or their combinations. For example, the processing unit may include a central processing unit (CPU) and/or a graphic processing unit (GPU). The integrated circuit may include a chip such as a field-programmable gate array (FPGA).

In some aspects, some or all operations in method 500 may be performed by the FPGAs. In aspects, when some operations are performed by FPGAs, the data after an operation performed by the FPGA may be communicated by the FPGAs to the CPUs so that the CPUs may perform subsequent operation(s) in method 500 using such data. In some aspects, all the operations in method 500 may be performed by CPUs. Alternatively, the operations performed by CPUs may be performed by other processors such as the dedicated processors, or GPUs.

In some embodiments, the methods 500 includes an operation 510 of tilting a sample stage of the optical system by a tilt angle, wherein the sample is immobilized on the sample stage. The tilting of the sample stage may be relative to the focal plane of objective, or otherwise the focal plane of the optical system when the optical system lacks any objective lens. In some embodiments, the sample may be immobilized on a flow cell, which is immobilized on the sample stage. In some embodiments, the sample may be a test target that simulates the presence of a flow cell with sample immobilized thereon. As disclosed herein, the sample may also be a beaded flow cell with sample(s) immobilized thereon.

In some alternative embodiments, the operation 510 may include tilting an image sensor or a dedicated AF sensor instead of the sample stage by the tilt angle to achieve equivalent effect on the image to be acquired in operation 520. Such tilting is relative to the focal plane of the objective lens or the focal plane of the optical system. However, tilting the sample stage may be preferred since tilting the image sensor in each autofocusing process and de-tilting it back for imaging after autofocusing is completed may introduce inconsistency or errors in the optical alignment of the image sensor to the other optical elements of the optical system, e.g., the corresponding tube lens. Tilting the AF sensor has the advantage that it does not need to be de-tilted back, and may remain tilted since it is dedicated for AF usage only. However, it may add additional cost and complexity to the optical system than those without AF sensors.

The sample stage may be motorized or otherwise connected to a motor so that tilting of the sample stage may occur automatically with prespecified angular resolution. In some embodiments, the tilting may occur after receiving an instruction by a user or a computer system disclosed herein. In some embodiments, tilting the sample stage of the optical system by the tilt angle is about a x or y axis. In some embodiments, tilting the sample stage of the optical system by the tilt angle is within a x-z plane or y-z plane. In some embodiments, tilting the AF sensor or image sensor of the optical system by the tilt angle is about x or y axis. In some embodiments, tilting the AF sensor or image sensor of the optical system by the tilt angle is within a x-z plane or y-z plane. The title angle may be in a range from 0.01 degrees to 89 degrees. The tilt angle may be in a range from 0.05 degrees to 15 degrees. The tilt angle may be in a range from 0.05 degrees to 8 degrees. In some embodiments, the title angle is clockwise about the x or y axis. In some embodiments, the title angle is counter-clockwise about the x or y axis. In some embodiments, the angular resolution of the tilting angle is in a range from 0.001 degrees to 0.2 degrees. In some embodiments, the angular resolution of the tilting angle is in a range from 0.01 degrees to 0.1 degrees. In some embodiments, the angular resolution of the tilting angle is in a range from 0.01 degrees to 0.08 degrees.

In some embodiments, the operation 510 comprises an operation of receiving, by a motor coupled to the sample stage, the tilt angle. The tilt angle can be included in an instruction received by the motor. Such instruction can be provided by a user, e.g., by entering the tilt angle at an input device of the optical system, or entered automatically by a computer system of the sequencing system 100.

In some embodiments, the operation 510 comprises an operation of tilting, by the motor, the sample stage automatically by the tilt angle. FIG. 6A shows schematics diagram of tilting the sample stage.

In embodiments with sequencing nucleic acids using the sequencing system 100 herein, the operation 510 of tilting the sample stage of the optical system by the tilt angle may be performed simultaneously as moving the sample stage within the x-y plane. In such embodiments, moving the sample stage within the x-y plane comprises moving the sample stage to a predetermined spatial location, e.g., from a current tile/subtile to a next tile or subtile of the flow cell relative to the objective lens so as to position the next tile or subtile with the imaging area of the objective lens. For example, after the first tile has been imaged, the sample stage moves within the x-y plane to position a second tile for imaging. During the movement in the x-y plane, the sample stage may also be tilted by the tilt angle so that tilting the sample stage adds minimal if any additional time to the existing sequencing time.

In some embodiments, the methods 500 comprises an operation 520 of obtaining, by the image sensor of the optical system, an image of the sample on the tilted sample stage.

In some embodiments, the method may further comprise an operation of conducting, by the sequencing system, a cycle of sequencing reactions before operation 520. In some embodiments, conducting, by the sequencing system, a cycle of sequencing reactions comprises: contacting nucleotide acid molecules immobilized on a support structure, e.g., a flow cell, with a plurality of sequencing primers, a plurality of polymerases and a mixture of different types of avidites. An individual avidite in the mixture may comprise a core attached with multiple nucleotide arms and each arm of the individual avidite comprises the same type of nucleotide base. In some embodiments, conducting, by the sequencing system, the cycle of sequencing reactions comprises: capturing optical color signals emitted from the nucleotide reagents that are bound to the nucleotide acid molecules by the image sensor.

In alternative embodiments, the operation 520 may be performed by an AF sensor instead of an image sensor. In embodiments that the sample stage is tilted in operation 510, then the un-tilted image sensor or un-tilted AF sensor can be used in operation 520 to obtain the image. In embodiments that the image sensor or AF sensor is tilted in operation 510, then the tilted image sensor or tilted AF sensor can be used in operation 520 to obtain the image, respectively.

A partly “out of focus” image 600 of the sample on the tilted sample stage as shown in the bottom right of FIG. 6A is shown in FIG. 6B. The image center 607 corresponds to the center O at the optical axis and in the x-y plane. Tilting of the sample stage causes a portion of the image to be dark because the sample corresponding to the dark region is out-of-focus. Only a small region 606 close to an edge of the image remains in-focus. The distance from the center 606a of the in-focus region 606 to the image center 607 can be the x-y plane shift. When the tilting is about y axis, the x-y plane shift corresponds to the x-shift in FIG. 6A. The size of the in-focus region may depend on the tilt angle, the depth of field, the image resolution, and/or other parameters of the optical system.

In some embodiments, the image 600 includes only a single image. In some embodiments, the image sensor is used for autofocusing the optical system and also for imaging sample(s) using the optical system. The image sensor may be an image sensor of any of the detection channels of the multi-channel fluorescent imaging module.

In some embodiments, the image 600 comprises a size along the x and/or y axis that is identical to a size along a corresponding axis of the image sensor or the AF sensor (if acquired by the AF sensor). When the tilt angle is about the y axis, the image size along the y axis is the same to the size of the image sensor along the y axis, while the size along the x axis can be different than the size of the image sensor along the y axis, e.g., smaller. For example, the size of the image 600 along x-axis is the same size of the image sensor along the x-axis. In some embodiment, an image that has a smaller width (along the y axis) than the image 600 may work equivalently as the image 600 in autofocusing but may advantageously save some computational time in processing the smaller imaging and/or storage space in storing the image.

In some embodiments, the image comprises a length or width that is in a range from 0.1 mm to 5 cm. In some embodiments, the image comprises a length (along the x-axis) or width (along the y axis) that is in a range from 0.1 mm to 30 mm. In some embodiments, the image comprises a length (along the x-axis) or width (along the y axis) that is in a range from 0.5 mm to 10 mm. In some embodiments, the image comprises a length (along the x-axis) or width (along the y axis) that is in a range from 0.5 mm to 5 mm.

The image may comprise various numbers of pixels along the x or y axis. For example, image 600 comprise a matrix size of about 5400×3600. In some embodiments, the image may comprise 60 to 60,000 pixels along x and/or y axis. In some embodiments, the image may comprise 600 to 8,000 pixels along x and/or y axis. In some embodiments, the matrix size may be optimized to achieve accurate determination of the center of the in-focus region 606a without increasing unnecessary imaging time and/or image processing time.

In some embodiment, in operation 520, the image 600 may be acquired by an AF sensor that is dedicated only for autofocusing purpose but not used for imaging after autofocusing is completed.

In some embodiments, the methods 500 comprises an operation 530 of determining, by a processor, a z shift. The z shift can be determined based on: (1) the tilt angle; and (2) a x-y plane shift from a center of the image. The x-y plane shift is determined based on an in-focus region of the image.

Referring back to FIG. 6A, in this particular embodiment, the sample stage is tilted by a tilt angle 609 along the x-axis, counterclockwise, in the x-z plane defined by the x and z axis. When the sample stage is in the focal plane of the objective, e.g., in focus, tilting about the center O, e.g., the intersecting point of the optical axis or z axis with the x axis, the in-focus region remains at the center O (bottom left of FIG. 6A). When the sample stage is not in the focal plane of the objective, e.g., out of focus, tilting about the center O causes an x-y plane shift, which is a shift along the x-axis of an in-focus region away from the center O (bottom right of FIG. 6A). The z shift 608 can be determined as:

$$( z shift ) = ( x - y plane shift ) * tan ( tilt angle )$$

where the z shift is the spatial shift along the z axis, tan( ) is the tangent function, the tilt angle is the angle that is tilted from the original position about an axis in the x-y plane, and the x-y plane shift is the spatial shift from the center of the in-focus area of the image to the center of the whole image.

In some embodiments, the x-y plane shift can be any shift within the x-y plane. In some embodiments, the x-y plane shift can be any shift along the x axis or y axis.

In some embodiments, the x-y plane shift is a distance from the image center that corresponds to intersecting point of the optical axis and the image plane (e.g., the x-y plane) to a center of an in-focus region. The center of the in-focus region can be on a straight line that is within the x-y plane. The center of the in-focus region can be on a straight line that that is orthogonal to the tilting axis of the tilt angle. For example, as shown in FIG. 6B, the center 606a of the in-focus region 606 is on x axis, which is within the x-y plane. The tilt angle, as shown in FIG. 6A is also about the x-axis, and the center 606a is on x-axis which is orthogonal to the y-axis which is the tilting axis of the tilt angle.

The center 606a of the in-focus region can be determined using various image processing algorithms. For example, the image can be separated in a predetermined number of regions, e.g., 20-40 regions, and the sum or average image intensity of each region can be used to identify the approximate location of the in-focus region because the in-focus region include a higher intensity than the out-of-focus dark regions. Subsequently, image intensity (e.g., intensity projection) and/or spatial frequency (Fourier transform of the intensities) information of the approximate in-focus region can be used to locate the center 606a. In some embodiments, information about the geometrical patterns in the image may be a factor in determining the image processing algorithm(s) for finding the center 606a.

In some embodiments, tilting along the x axis or y axis may be simpler or more convenient to implement than tilting in other directions within the x-y plane. Determining an x-y plane shift along the x or y axis, as a result of such tilting along the x or y axis, can be computationally more convenient and efficient.

In some embodiments, the methods 500 can include an operation 540 of moving the sample stage relative to the focal plane of an objective lens of the optical system by the determined z shift thereby autofocusing the optical system. After determining the z shift in operation 530, the sample stage can be moved relative to the focal plane of the objective lens to bring the sample in focus. This relative movement may be achieved by keeping the sample stage in the same position relative to the baseplate (205 in FIGS. 2A-2B) and moving the objective lens relative to the baseplate, thereby moving the focal plane closer and substantially overlapping with the sample stage (e.g., within ±200 nm or ±100 nm). Alternatively, the sample stage can be moved with respect to the baseplate by the z shift, while the objective lens and its focal plane remain at the same spatial location with respect to the baseplate.

As disclosed herein, the sample stage may be motorized or otherwise connected to a motor so that movement of the sample stage can be automatic in response to receiving an instruction either provided by a user or by a computer system disclosed herein. Similarly, the objective lens may be coupled to a motorized stage, e.g., z-stage, so that its movement can be similarly controlled and be automatic in response to receiving an instruction by a user or a computer system.

In some embodiments, the methods 500 include an operation of de-tilting the tilted sample stage, the tilted image sensor, or the tilted AF sensor that is tilted in operation 510 back by the tilt angle (in an opposite direction) to return it to the spatial position before the tilting operation 510.

In embodiments with sequencing nucleic acids using the sequencing system 100 herein, the operation of de-tilting the tilted sample stage, the tilted AF sensor, or the tilted image sensor back to the position before the tilting operation of 510 may be performed at least partly simultaneously to the operation 540 of moving the sample stage relative to the focal plane of the objective lens by the determined z shift. The de-tilting and moving the sample stage in focus by operation 540 may be performed simultaneously to reduce time needed for focusing the sample, thus total time for sequencing. In some embodiments, the AF sensor can remain tilted and does not need to be de-tilted back.

In some embodiments, the methods 500 herein can be used for autofocusing along the z axis. In some embodiments, a z shift of the objective lens relative to the sample is determined in order to place the sample in the focal plane of the objective lens. In some embodiments, an error in autofocusing the optical system is in the range from −400 nm to +400 nm. In some embodiments, an error in autofocusing the optical system is in the range from −200 nm to +200 nm. In some embodiments, an error in autofocusing the optical system is in the range from −100 nm to +100 nm. In some embodiments, an error in autofocusing the optical system is in the range from −50 nm to +50 nm.

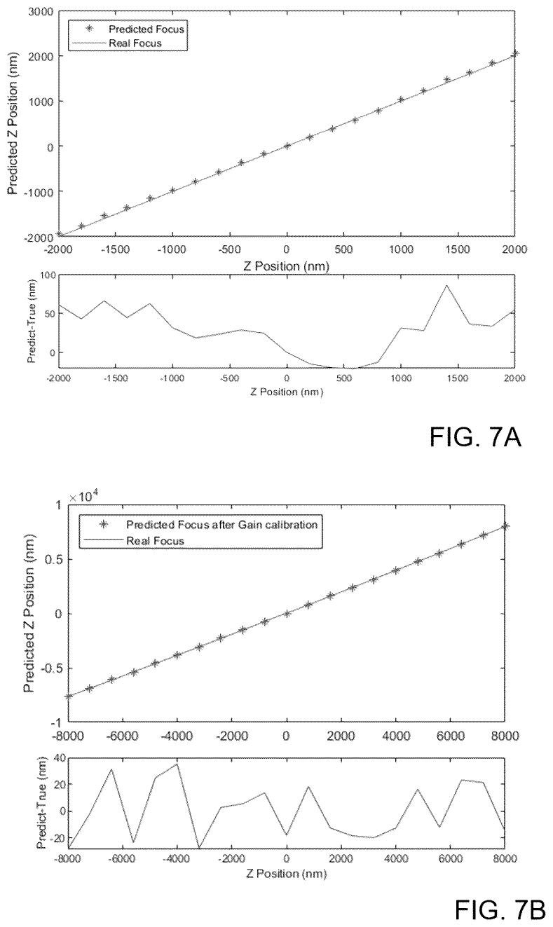

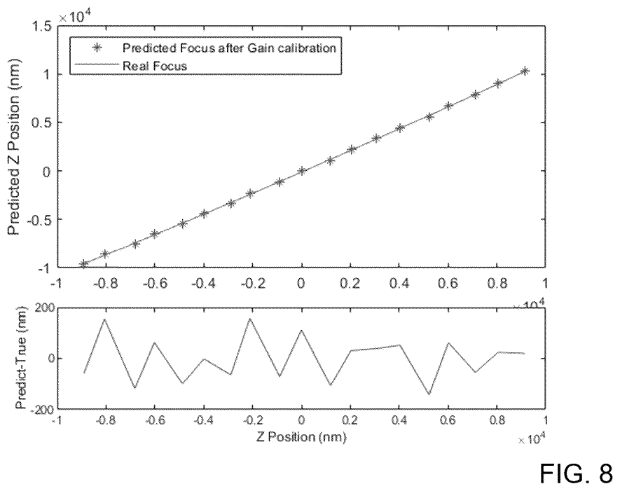

FIGS. 7A-7B and FIG. 8 show z shifts determined using the systems and methods herein in comparison with actual z shifts that are generated by directly moving the objective lens relative to the sample stage out-of-focus with the predetermined z shifts. FIG. 7A shows the difference between estimated z shifts by tilting the sample stage while keeping the image sensor fixed relative to the baseplate using the methods herein and the actual z shift. The differences are less than ±100 nm or even less than ±50 nm at all the different z locations. The tilt angle is 0.2 degrees in FIG. 7A and 0.8 degrees in FIG. 7B. FIG. 8 shows the difference between estimated z shifts by tilting the image sensor using the methods herein and the actual z shift. The tilt angle in FIG. 8 is 3 degrees. The estimated z shifts are isolated points in FIGS. 7A-7B and FIG. 8. Various types of fitting can be used to fit the isolated points. For example, first order and/or second order polynomial fittings may be used. The difference at the isolated estimated points and at other points therebetween are shown in the bottom panels of FIGS. 7A-7B and FIG. 8. The differences at points between the isolated points are estimated using the fitted line. As mentioned above, the fitted line can be generated using various fitting methods, and the difference may be obtained as the optimal fitting results. Alternatively, fitting may be limited to certain algorithms, e.g., polynomial fitting up to the third order, in determining the differences between the estimated z shift and the actual z shift.

In some embodiments, autofocus using the methods 500 disclosed herein of the optical system can be completed within 50 to 1200 milliseconds. In some embodiments, autofocus using the methods disclosed herein of the optical system can be completed within 50 to 990 milliseconds. In some embodiments, autofocus using the methods disclosed herein of the optical system can be completed in less than 400, 500, or 600 milliseconds.

### Pivot Point Calibration

In some embodiments, the methods 500 herein include an operation of calibrating a pivot point of the optical system. Such operation can be performed before any autofocusing operation of 510-540. The operation of calibration of the pivot point can be used to determine whether the pivot point is along the optical axis of the optical system or not. The pivot point after calibration is the vertex of the tilt angle during autofocusing (e.g., the center O in FIG. 6A). The operation of calibration the pivot point can be performed when needed. For example, it may be performed only once before a sequencing run starts, and it does not need to be repeated for different flow cycles. As another example, it may be performed during a sequencing run in specific flow cycle(s).

In some embodiments, the operation of calibrating the pivot point of the optical system comprises an operation of tilting the sample stage, the image sensor, or the AF sensor. In some embodiments, the sample stage, image sensor, or AF sensor is in-focus along z before the calibration operation. The tilt angle for calibration can be identical to the tilt angle that will be used in autofocusing operations 510. Alternatively, the tilt angle for calibration may be a second tilt angle different from the tilt angle in operation 510. In some embodiments, the operation of calibrating the pivot point of the optical system comprises an operation of acquiring a calibration image of the sample immobilized on the sample stage.

FIG. 9 shows an example calibration image of a test target as the sample immobilized on the sample stage. The calibration image can be acquired by either an image sensor or a dedicated AF sensor. Calibrating the pivot point can include an operation of determining a pivot point offset based on a regional center of an in-focus region of the calibration image and an image center of the calibration image. The pivot point offset can be from the optical axis 1099. Referring to FIG. 9, the pivot point offset is the distance between the center of the image 1099 that corresponds to the optical axis intersection point with the x-y plane or the image plane and the regional center 1091 of the in-focus region. Without pivot point offset, the center of the image 1099 may overlap with the center of the in-focus region 1091. With the pivot point offset, the center of the in-focus region may shift away from the center of the image after tilting. The regional center can be determined using various image processing algorithms that can be used in operation 530. After the pivot point offset is determined, the calibration operation can include de-tilting the sample stage, the image sensor, or the AF sensor back into the position before the calibration operation starts.

In some embodiments, the determined pivot point offset may be considered in operation 530 to determine the x-y plane shift. In some embodiments, the operation 530 may comprise an operation of subtracting the determined pivot point offset from the x-y plane shift to obtain the x-y plane shift' so that the z shift may be calculated as

(z shift)=[(x-y plane shift)−(pivot point offset)]*tan(tilt angle).

### Computer Systems

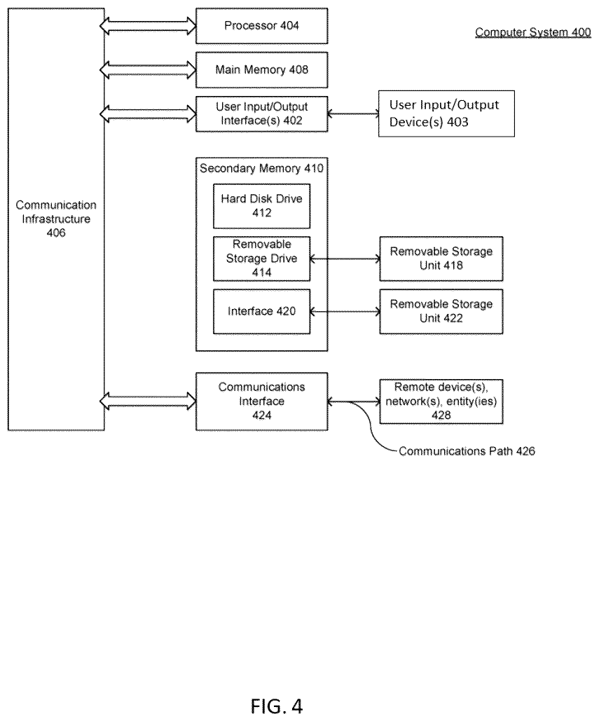

FIG. 4 illustrates a block diagram of a computer system for autofocusing, according to some embodiments. Various aspects of the methods described herein, such as methods 500, as well as combinations and sub-combinations thereof, may be implemented, for example, using one or more computer systems, such as computer system 400 shown in FIG. 4.

The computer system 126 in FIG. 1 may include one or more computer systems 400. The computer system 400 may include one or more hardware processors 404. The hardware processor 404 may include a central processing unit (CPU), graphic processing units (GPU), or their combination. Processor 404 may be connected to a bus or communication infrastructure 406.

Computer system 400 may also include user input/output device(s) 403, such as monitors, keyboards, pointing devices, etc., which may communicate with communication infrastructure 406 through user input/output interface(s) 402. The user input/output devices 403 may be coupled to the user interface 124 in FIG. 1.

One or more of processors 404 may be a graphics processing unit (GPU). In an aspect, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, vector processing, array processing, etc., as well as cryptography (including brute-force cracking), generating cryptographic hashes or hash sequences, solving partial hash-inversion problems, and/or producing results of other proof-of-work computations for some blockchain-based applications, for example. With capabilities of general-purpose computing on graphics processing units (GPGPU), the GPU may be particularly useful in at least the image recognition and machine learning aspects described herein.

Additionally, one or more of processors 404 may include a coprocessor or other implementation of logic for accelerating cryptographic calculations or other specialized mathematical functions, including hardware-accelerated cryptographic coprocessors. Such accelerated processors may further include instruction set(s) for acceleration using coprocessors and/or other logic to facilitate such acceleration.

Computer system 400 may also include a data storage device such as a main or primary memory 408, e.g., random access memory (RAM). Main memory 408 may include one or more levels of cache. Main memory 408 may have stored therein control logic (e.g., computer software) and/or data.

Computer system 400 may also include one or more secondary data storage devices or secondary memory 410. Secondary memory 410 may include, for example, a main storage drive 412 and/or a removable storage device or drive 414. Main storage drive 412 may be a hard disk drive or solid-state drive, for example. Removable storage drive 414 may be a floppy disk drive, a magnetic tape drive, a compact disk drive, an optical storage device, tape backup device, and/or any other storage device/drive.

Removable storage drive 414 may interact with a removable storage unit 418.

Removable storage unit 418 may include a computer usable or readable storage device having stored thereon computer software and/or data. The software may include control logic. The software may include instructions executable by the hardware processor(s) 404. Removable storage unit 418 may be a floppy disk, magnetic tape, compact disk, DVD, optical storage disk, and/any other computer data storage device. Removable storage drive 414 may read from and/or write to removable storage unit 418.

Secondary memory 410 may include other methods, devices, components, instrumentalities, or other approaches for allowing computer programs and/or other instructions and/or data to be accessed by computer system 400. Such methods, devices, components, instrumentalities, or other approaches may include, for example, a removable storage unit 422 and an interface 420. Examples of the removable storage unit 422 and the interface 420 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 400 may further include a communication or network interface 424. Communication interface 424 may enable computer system 400 to communicate and interact with any combination of external devices, external networks, external entities, etc. (individually and collectively referenced by reference number 428). For example, communication interface 424 may allow computer system 400 to communicate with external or remote devices 428 over communication path 426, which may be wired and/or wireless (or a combination thereof), and which may include any combination of LANs, WANs, the Internet, etc. Control logic and/or data may be transmitted to and from computer system 400 via communication path 426. In some aspects, communication path 426 is the connection to the cloud 130, as depicted in FIG. 1. The external devices, etc. referred to by reference number 428 may be devices, networks, entities, etc. in the cloud 130.

Computer system 400 may also be any of a personal digital assistant (PDA), desktop workstation, laptop or notebook computer, netbook, tablet, smart phone, smart watch or other wearable, appliance, part of the Internet of Things (IoT), and/or embedded system, to name a few non-limiting examples, or any combination thereof.

It should be appreciated that the framework described herein may be implemented as a method, process, apparatus, system, or article of manufacture such as a non-transitory computer-readable medium or device.

Computer system 400 may be a client or server, accessing or hosting any applications and/or data through any delivery paradigm, including but not limited to remote or distributed cloud computing solutions; local or on-premises software (e.g., “on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), database as a service (DBaaS), etc.); and/or a hybrid model including any combination of the foregoing examples or other services or delivery paradigms.

Any applicable data structures, file formats, and schemas may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

Any pertinent data, files, and/or databases may be stored, retrieved, accessed, and/or transmitted in human-readable formats such as numeric, textual, graphic, or multimedia formats, further including various types of markup language, among other possible formats. Alternatively or in combination with the above formats, the data, files, and/or databases may be stored, retrieved, accessed, and/or transmitted in binary, encoded, compressed, and/or encrypted formats, or any other machine-readable formats.

Interfacing or interconnection among various systems and layers may employ any number of mechanisms, such as any number of protocols, programmatic frameworks, floorplans, or application programming interfaces (API), including but not limited to Document Object Model (DOM), Discovery Service (DS), NSUserDefaults, Web Services Description Language (WSDL), Message Exchange Pattern (MEP), Web Distributed Data Exchange (WDDX), Web Hypertext Application Technology Working Group (WHATWG) HTML5 Web Messaging, Representational State Transfer (REST or RESTful web services), Extensible User Interface Protocol (XUP), Simple Object Access Protocol (SOAP), XML Schema Definition (XSD), XML Remote Procedure Call (XML-RPC), or any other mechanisms, open or proprietary, that may achieve similar functionality and results.

Such interfacing or interconnection may also make use of uniform resource identifiers (URI), which may further include uniform resource locators (URL) or uniform resource names (URN). Other forms of uniform and/or unique identifiers, locators, or names may be used, either exclusively or in combination with forms such as those set forth above.

Any of the above protocols or APIs may interface with or be implemented in any programming language, procedural, functional, or object-oriented, and may be compiled or interpreted. Non-limiting examples include C, C++, C#, Objective-C, Java, Scala, Clojure, Elixir, Swift, Go, Perl, PHP, Python, Ruby, JavaScript, WebAssembly, or virtually any other language, with any other libraries or schemas, in any kind of framework, runtime environment, virtual machine, interpreter, stack, engine, or similar mechanism, including but not limited to Node.js, V8, Knockout, jQuery, Dojo, Dijit, OpenUI5, AngularJS, Expressjs, Backbone.js, Ember.js, DHTMLX, Vue, React, Electron, and so on, among many other non-limiting examples.

In some aspects, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 400, main memory 408, secondary memory 410, and removable storage units 418 and 422, as well as tangible articles of manufacture embodying any combination of the foregoing. Such control logic, when executed by one or more data processing devices (such as computer system 400), may cause such data processing devices to operate as described herein.

Based on the teachings contained in this disclosure, it will be apparent to persons skilled in the relevant art(s) how to make and use aspects of this disclosure using data processing devices, computer systems and/or computer architectures other than that shown in FIG. 4. In particular, aspects may operate with software, hardware, and/or operating system implementations other than those described herein.

### Supports and Low Non-Specific Coatings

In some embodiments, NGS sequencing compositions and methods, e.g., pairwise sequencing, employ a support comprising a plurality of oligonucleotide surface primers immobilized thereon. In some embodiments, the support is passivated with a low non-specific binding coating. The surface coatings described herein exhibit very low non-specific binding to reagents typically used for nucleic acid capture, amplification and sequencing workflows, such as dyes, nucleotides, enzymes, and nucleic acid primers. The surface coatings exhibit low background fluorescence signals or high contrast-to-noise (CNR) ratios compared to conventional surface coatings.

The low non-specific binding coating comprises one layer or multiple layers (FIG. 11). In some embodiments, the plurality of surface primers is immobilized to the low non-specific binding coating. In some embodiments, at least one surface primer is embedded within the low non-specific binding coating. The low non-specific binding coating enables improved nucleic acid hybridization and amplification performance. In general, the supports comprise a substrate (or support structure), one or more layers of a covalently or non-covalently attached low-binding, and chemical modification layers, e.g., silane layers, polymer films, and one or more covalently or non-covalently attached surface primers that can be used for tethering single-stranded nucleic acid library molecules to the support. In some embodiments, the formulation of the coating, e.g., the chemical composition of one or more layers, the coupling chemistry used to cross-link the one or more layers to the support and/or to each other, and the total number of layers, may be varied such that non-specific binding of proteins, nucleic acid molecules, and other hybridization and amplification reaction components to the coating is minimized or reduced relative to a comparable monolayer. The formulation of the coating described herein may be varied such that non-specific hybridization on the coating is minimized or reduced relative to a comparable monolayer. The formulation of the coating may be varied such that non-specific amplification on the coating is minimized or reduced relative to a comparable monolayer. The formulation of the coating may be varied such that specific amplification rates and/or yields on the coating are maximized. Amplification levels suitable for detection are achieved in no more than 2, 3, 4, 5, 6, 7, 8, 9, 10, 15, 20, 25, 30, or more than 30 amplification cycles in some cases disclosed herein.

The support structure that comprises the one or more chemically-modified layers, e.g., layers of a low non-specific binding polymer, may be independent or integrated into another structure or assembly. For example, in some embodiments, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments, the support structure comprises the interior surface (such as the lumen surface) of a capillary etched into a planar chip.

The attachment chemistry used to graft a first chemically-modified layer to the surface of the support will generally be dependent on both the material from which the surface is fabricated and the chemical nature of the layer. In some embodiments, the first layer may be covalently attached to the surface. In some embodiments, the first layer may be non-covalently attached, e.g., adsorbed to the support through non-covalent interactions such as electrostatic interactions, hydrogen bonding, or van der Waals interactions between the support and the molecular components of the first layer. In either case, the support may be treated prior to attachment or deposition of the first layer. Any of a variety of surface preparation techniques known to those of skill in the art may be used to clean or treat the surface. For example, glass or silicon surfaces may be acid-washed using a Piranha solution (a mixture of sulfuric acid (H2SO4) and hydrogen peroxide (H2O2)), base treatment in KOH and NaOH, and/or cleaned using an oxygen plasma treatment method.

Silane chemistries constitute non-limiting approaches for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding coatings include, but are not limited to, (3-Aminopropyl) trimethoxysilane (APTMS), (3-Aminopropyl)triethoxysilane (APTES), any of a variety of PEG-silanes (e.g., comprising molecular weights of 1K, 2K, 5K, 10K, 20K, etc.), amino-PEG silane (e.g., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the support, where the choice of components used may be varied to alter one or more properties of the layers, e.g., the surface density of functional groups and/or tethered oligonucleotide primers, the hydrophilicity/hydrophobicity of the layers, or the three three-dimensional nature (e.g., “thickness”) of the layer. Examples of polymers that may be used to create one or more layers of low non-specific binding material in any of the disclosed coatings include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag-Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the support. Alternatively, the surface coating may be patterned, such that the chemical modification layers are confined to one or more discrete regions of the support. For example, the coating may be patterned using photolithographic techniques to create an ordered array or random pattern of chemically-modified regions on the support. Alternately or in combination, the coating may be patterned using, e.g., contact printing and/or ink-jet printing techniques. In some embodiments, an ordered array or random pattern of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In some embodiments, the low nonspecific binding coatings comprise hydrophilic polymers that are non-specifically adsorbed or covalently grafted to the support. Passivation may be performed utilizing poly(ethylene glycol) (PEG, also known as polyethylene oxide (PEO) or polyoxyethylene) or other hydrophilic polymers with different molecular weights and end groups that are linked to a support using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface. In some embodiments, two or more layers may be covalently coupled to each other or internally cross-linked to improve the stability of the resulting coating. In some embodiments, surface primers with different nucleotide sequences and/or base modifications (or other biomolecules, e.g., enzymes or antibodies) may be tethered to the resulting layer at various surface densities. In some embodiments, for example, both surface functional group density and surface primer concentration may be varied to attain a desired surface primer density range. Additionally, surface primer density can be controlled by diluting the surface primers with other molecules that carry the same functional group. For example, amine-labeled surface primers can be diluted with amine-labeled polyethylene glycol in a reaction with an NHS-ester coated surface to reduce the final primer density. Surface primers with different lengths of linker between the hybridization region and the surface attachment functional group can also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In some embodiments, the low nonspecific binding coatings comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl) acrylamide-co-acrylamide (PAZAM).

In order to scale primer surface density and add additional dimensionality to hydrophilic or amphoteric coatings, supports comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the support significantly. Traditional PEG coating approaches use monolayer primer deposition, which has been generally reported for single molecule applications, but does not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some embodiments, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some embodiments, high primer density materials may be constructed in solution and subsequently layered onto the surface in multiple operations.

Examples of materials from which the support structure may be fabricated include, but are not limited to, glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic support structures are contemplated.

The support structure may be rendered in any of a variety of geometries and dimensions known to those of skill in the art, and may comprise any of a variety of materials known to those of skill in the art. For example, the support structure may be locally planar (e.g., comprising a microscope slide or the surface of a microscope slide). Globally, the support structure may be cylindrical (e.g., comprising a capillary or the interior surface of a capillary), spherical (e.g., comprising the outer surface of a non-porous bead), or irregular (e.g., comprising the outer surface of an irregularly-shaped, non-porous bead or particle). In some embodiments, the surface of the support structure used for nucleic acid hybridization and amplification may be a solid, non-porous surface. In some embodiments, the surface of the support structure used for nucleic acid hybridization and amplification may be porous, such that the coatings described herein penetrate the porous surface, and nucleic acid hybridization and amplification reactions performed thereon may occur within the pores.

The support structure that comprises the one or more chemically-modified layers, e.g., layers of a low non-specific binding polymer, may be independent or integrated into another structure or assembly. For example, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments the support structure comprises the interior surface (such as the lumen surface) of a capillary etched into a planar chip.

As noted, the low non-specific binding supports of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, exposure of the surface to fluorescent dyes (e.g., cyanins such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging, may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations-provided that care has been taken to ensure that the fluorescence imaging is performed under conditions where fluorescence signal is linearly related (or related in a predictable manner) to the number of fluorophores on the support surface (e.g., under conditions where signal saturation and/or self-quenching of the fluorophore is not an issue) and suitable calibration standards are used. In some embodiments, other techniques known to those of skill in the art, for example, radioisotope labeling and counting methods, may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some embodiments, the label may comprise a fluorescent label. In some embodiments, the label may comprise a radioisotope. In some embodiments, the label may comprise any other detectable label known to one of skill in the art. In some embodiments, the degree of non-specific binding exhibited by a given support surface formulation may thus be assessed in terms of the number of non-specifically bound protein molecules (or nucleic acid molecules or other molecules) per unit area. In some embodiments, the low-binding supports of the present disclosure may exhibit non-specific protein binding (or non-specific binding of other specified molecules, (e.g., cyanins such as Cy3, or Cy5, etc., fluoresceins, coumarins, rhodamines, etc. or other dyes disclosed herein)) of less than 0.001 molecule per μm², less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². Those of skill in the art will realize that a given support surface of the present disclosure may exhibit non-specific binding falling anywhere within this range, for example, of less than 86 molecules per μm². For example, some modified surfaces disclosed herein exhibit nonspecific protein binding of less than 0.5 molecule/μm² following contact with a 1 μM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm². In independent nonspecific binding assays, 1 μM labeled Cy3 SA (ThermoFisher), 1 μM Cy5 SA dye (ThermoFisher), 10 μM Aminoallyl-dUTP-ATTO-647N (Jena Biosciences), 10 μM Aminoallyl-dUTP-ATTO-Rhol 1 (Jena Biosciences), 10 μM Aminoallyl-dUTP-ATTO-Rhol 1 (Jena Biosciences), 10 μM 7-Propargylamino-7-deaza-dGTP-Cy5 (Jena Biosciences, and 10 μM 7-Propargylamino-7-deaza-dGTP-Cy3 (Jena Biosciences) were incubated on the low binding coated supports at 37° C. for 15 minutes in a 384 well plate format. Each well was rinsed 2-3× with 50 ul deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (e.g., inverted fluorescence microscope) (Olympus Corp., Center Valley, Pa.) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, N.Y.), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm². In some embodiments, the coated support was immersed in a buffer (e.g., 25 mM ACES, pH 7.4) while the image was acquired.

In some embodiments, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some embodiments, the hydrophilic surfaces disclosed herein facilitate reduced wash times for bioassays, often due to reduced nonspecific binding of biomolecules to the low-binding surfaces. In some embodiments, adequate wash operations may be performed in less than 60, 50, 40, 30, 20, 15, 10, or less than 10 seconds. For example, adequate wash operations may be performed in less than 30 seconds.

Some low-binding surfaces of the present disclosure exhibit significant improvement in stability or durability to prolonged exposure to solvents and elevated temperatures, or to repeated cycles of solvent exposure or changes in temperature. For example, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer may be attached or tethered to the support surface. In some embodiments, the one or more types of adapters or primers may comprise spacer sequences, adapter sequences for hybridization to adapter-ligated target library nucleic acid sequences, forward amplification primers, reverse amplification primers, sequencing primers, and/or molecular barcoding sequences, or any combination thereof. In some embodiments, 1 primer or adapter sequence may be tethered to at least one layer of the surface. In some embodiments, at least 2, 3, 4, 5, 6, 7, 8, 9, 10, or more than 10 different primer or adapter sequences may be tethered to at least one layer of the surface.

In some embodiments, the tethered adapter and/or primer sequences may range in length from about 10 nucleotides to about 100 nucleotides. In some embodiments, the tethered adapter and/or primer sequences may be at least 10, at least 20, at least 30, at least 40, at least 50, at least 60, at least 70, at least 80, at least 90, or at least 100 nucleotides in length. In some embodiments, the tethered adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some embodiments the length of the tethered adapter and/or primer sequences may range from about 20 nucleotides to about 80 nucleotides. Those of skill in the art will recognize that the length of the tethered adapter and/or primer sequences may have any value within this range, e.g., about 24 nucleotides.

In some embodiments, the resultant surface density of primers (e.g., capture primers) on the low binding support surfaces of the present disclosure may range from about 100 primer molecules per μm² to about 100,000 primer molecules per μm². In some embodiments, the resultant surface density of primers on the low binding support surfaces of the present disclosure may range from about 1,000 primer molecules per μm² to about 1,000,000 primer molecules per μm². In some embodiments, the surface density of primers may be at least 1,000, at least 10,000, at least 100,000, or at least 1,000,000 molecules per μm². In some embodiments, the surface density of primers may be at most 1,000,000, at most 100,000, at most 10,000, or at most 1,000 molecules per μm². Any of the lower and upper values described in this paragraph may be combined to form a range included within the present disclosure, for example, in some embodiments the surface density of primers may range from about 10,000 molecules per μm² to about 100,000 molecules per μm². Those of skill in the art will recognize that the surface density of primer molecules may have any value within this range, e.g., about 455,000 molecules per μm². In some embodiments, the surface density of target library nucleic acid sequences initially hybridized to adapter or primer sequences on the support surface may be less than or equal to that indicated for the surface density of tethered primers. In some embodiments, the surface density of clonally-amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000/μm², while also comprising at least a second region having a substantially different local density.

### Contrast to Noise Ratio (CNR)

In some embodiments, the performance of nucleic acid hybridization and/or amplification reactions using the disclosed reaction formulations and low-binding supports may be assessed using fluorescence imaging techniques, where the contrast-to-noise ratio (CNR) of the images provides a key metric in assessing amplification specificity and non-specific binding on the support. CNR is commonly defined as: CNR=(Signal-Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times must be minimized), as shown in the example below. At high CNR the imaging time required to reach accurate discrimination (and thus accurate base-calling in the case of sequencing applications) can be drastically reduced even with moderate improvements in CNR. Improved CNR in imaging data on the imaging integration time provides a method for more accurately detecting features such as clonally-amplified nucleic acid colonies on the support surface.

In most ensemble-based sequencing approaches, the background term is typically measured as the signal associated with ‘interstitial’ regions. In addition to “interstitial” background (Binter), “intrastitial” background (Bintra) exists within the region occupied by an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Binter background signal arises from a variety of sources; a few examples include auto-fluorescence from consumable flow cells, non-specific adsorption of detection molecules that yield spurious fluorescence signals that may obscure the signal from the ROI, and the presence of non-specific DNA amplification products (e.g., those arising from primer dimers). In many next generation sequencing (NGS) applications, this background signal in the current field-of-view (FOV) is averaged over time and subtracted. The signal arising from individual DNA colonies (e.g., (Signal)-B(interstial) in the FOV) yields a discernable feature that can be classified. In some embodiments, the intrastitial background (B(intrastitial)) can contribute a confounding fluorescence signal that is not specific to the target of interest, but is present in the same ROI thus making it far more difficult to average and subtract.

Nucleic acid amplification on the low-binding coated supports described herein may decrease the B(interstitial) background signal by reducing non-specific binding, may lead to improvements in specific nucleic acid amplification, and may lead to a decrease in non-specific amplification that can impact the background signal arising from both the interstitial and intrastitial regions. In some embodiments, the disclosed low-binding coated supports, optionally used in combination with the disclosed hybridization and/or amplification reaction formulations, may lead to improvements in CNR by a factor of 2, 5, 10, 100, 250, 500 or 1000-fold over those achieved using conventional supports and hybridization, amplification, and/or sequencing protocols.

Although described here in the context of using fluorescence imaging as the read-out or detection mode, the same principles apply to the use of the disclosed low-binding coated supports and nucleic acid hybridization and amplification formulations for other detection modes as well, including both optical and non-optical detection modes.

### Methods for Sequencing

The present disclosure provides methods for autofocusing optical systems that can be used for sequencing immobilized or non-immobilized template nucleotide acid molecules. In some embodiments, the immobilized template molecules comprise a plurality of nucleic acid template molecules having one copy of a target sequence of interest. In some embodiments, nucleic acid template molecules having one copy of a target sequence of interest can be generated by conducting bridge amplification using linear library molecules. In some embodiments, the immobilized template molecules comprise a plurality of nucleic acid template molecules each having two or more tandem copies of a target sequence of interest (e.g., concatemers). In some embodiments, nucleic acid template molecules comprising concatemer molecules can be generated by conducting rolling circle amplification of circularized linear library molecules. In some embodiments, the non-immobilized template molecules comprise circular molecules. In some embodiments, methods for sequencing employ soluble (e.g., non-immobilized) sequencing polymerases or sequencing polymerases that are immobilized to a support.

In some embodiments, the sequencing reactions employ detectably labeled nucleotide analogs. In some embodiments, the sequencing reactions employ a two-stage sequencing reaction comprising binding detectably labeled multivalent molecules, and incorporating nucleotide analogs. In some embodiments, the sequencing reactions employ non-labeled nucleotide analogs. In some embodiments, the sequencing reactions employ phosphate chain labeled nucleotides.

### Multivalent Molecules

The present disclosure provides methods for autofocusing optical systems that can be used for sequencing template nucleic acid molecules. In some embodiments, the sample immobilized or otherwise positioned on the support may include at least one multivalent molecule. In some embodiments, the sample that is used for autofocusing the optical system may include at least one multivalent molecule. In some embodiments, the sequencing methods utilizing the optical system for imaging may employ at least one multivalent molecule. In some embodiments, the sequencing methods utilizing the optical system for imaging may include autofocusing of the optical system before imaging one or more surfaces in a flow cycle of the sequencing run.

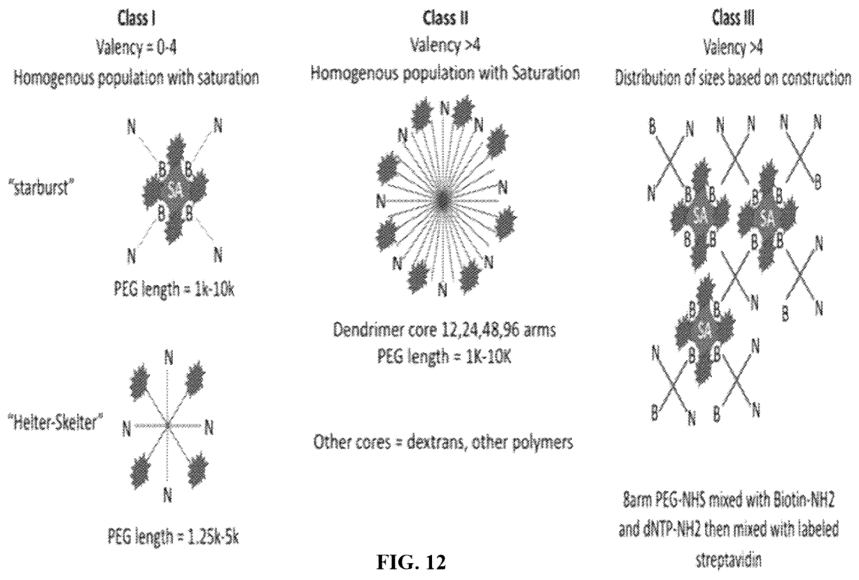

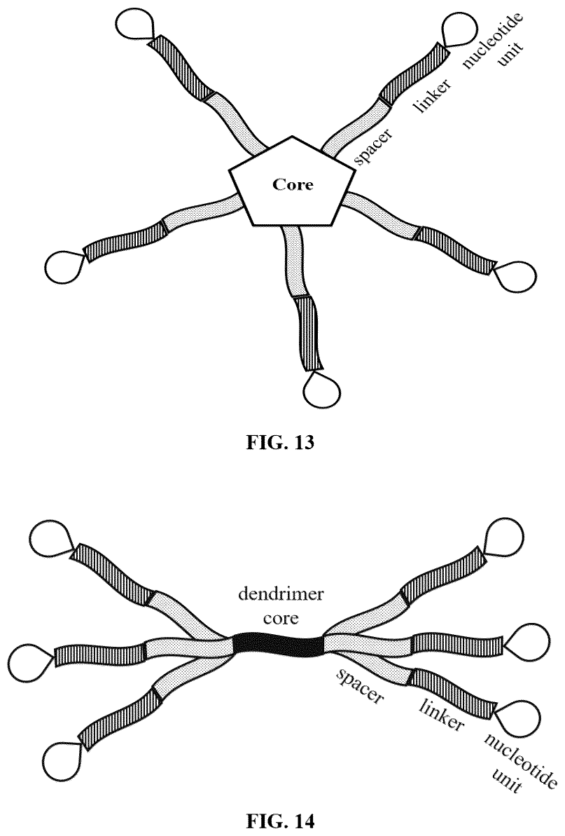

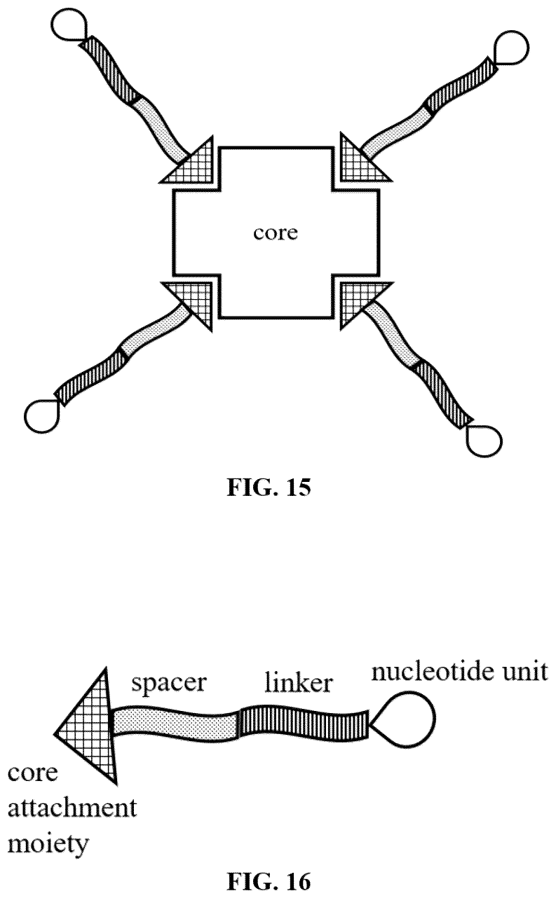

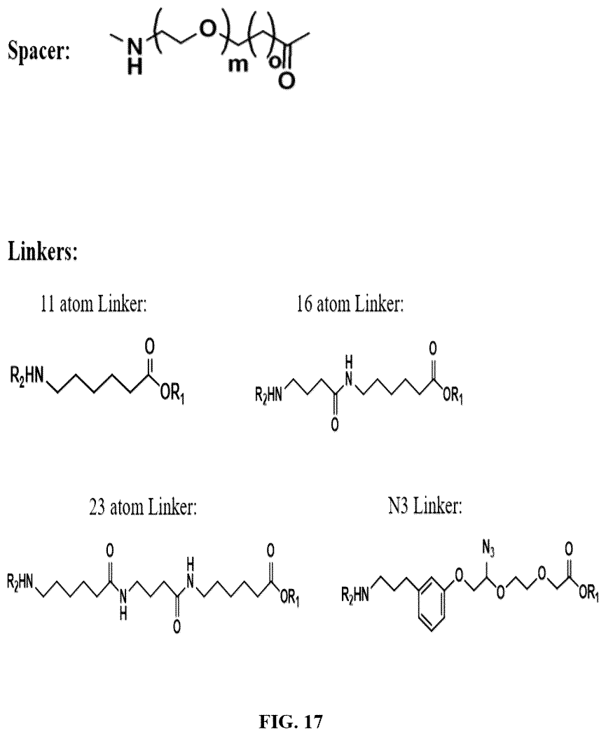

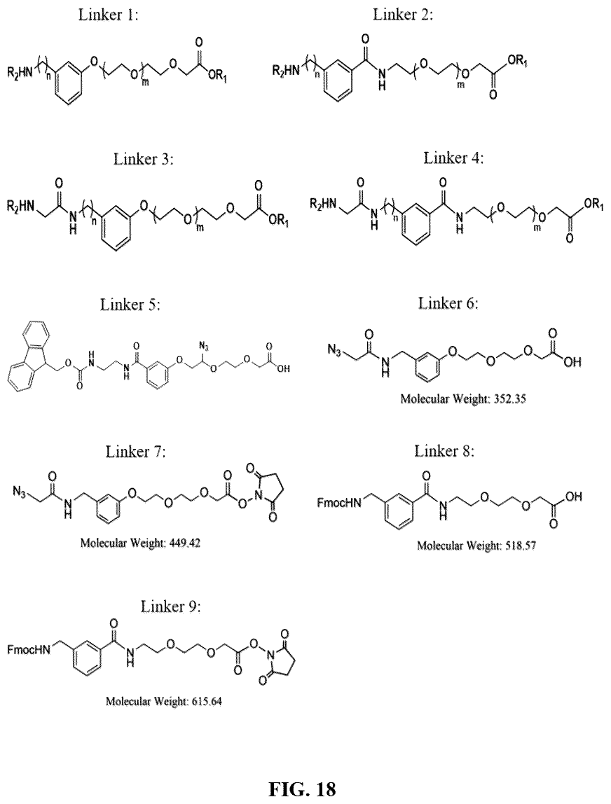

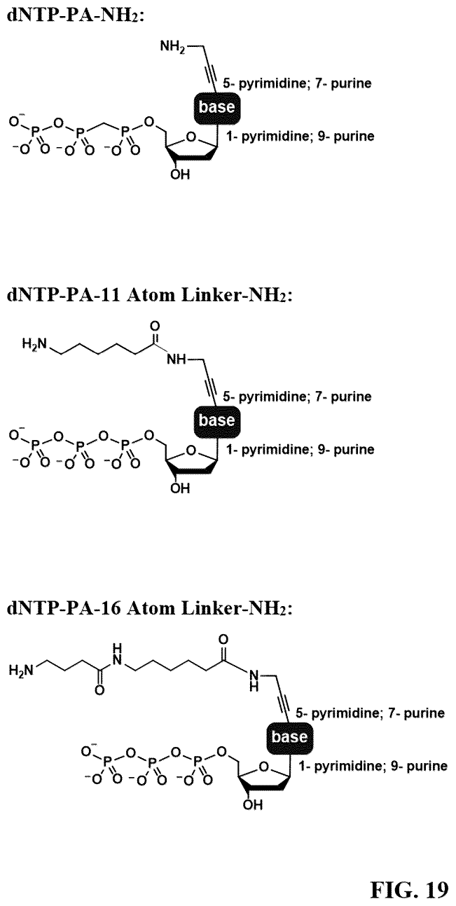

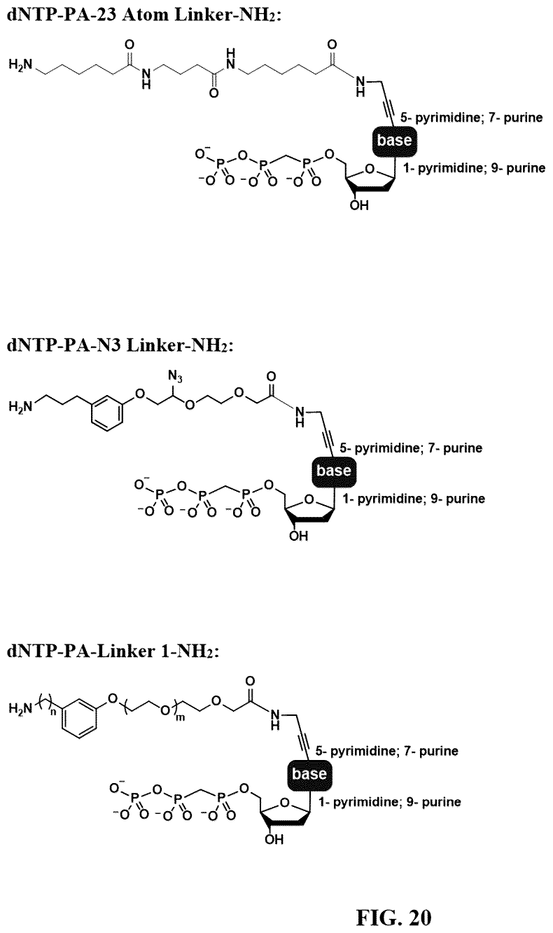

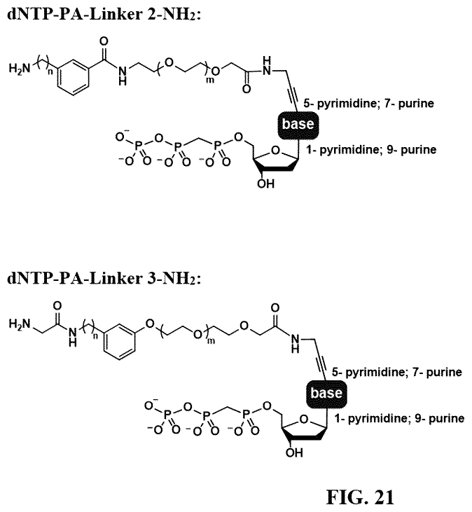

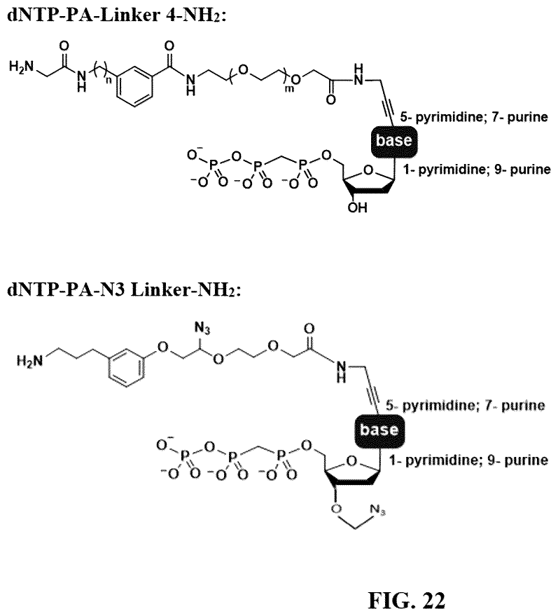

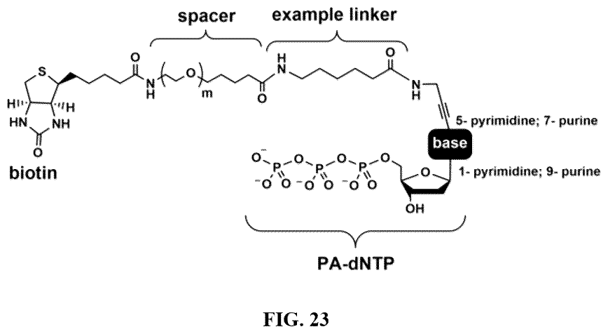

In some embodiments, the multivalent molecule comprises a plurality of nucleotide arms attached to a core and having any configuration including a starburst, helter skelter, or bottle brush configuration (e.g., FIG. 12). The multivalent molecule comprises: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, wherein the linker is attached to the nucleotide unit. In some embodiments, the nucleotide unit comprises a base, sugar and at least one phosphate group, and the linker is attached to the nucleotide unit through the base. In some embodiments, the linker comprises an aliphatic chain or an oligo ethylene glycol chain where both linker chains having 2-6 subunits. In some embodiments, the linker also includes an aromatic moiety. An example of a nucleotide arm is shown in FIG. 16. Examples of multivalent molecules are shown in FIGS. 12-15. An example of a spacer is shown in FIG. 17 (top) and examples of linkers are shown in FIG. 17 (bottom) and FIG. 18. Examples of nucleotides attached to a linker are shown in FIG. 19-22. An example of a biotinylated nucleotide arm is shown in FIG. 23.

In some embodiments, a multivalent molecule comprises a core attached to multiple nucleotide arms, and wherein the multiple nucleotide arms have the same type of nucleotide unit which is selected from a group consisting of dATP, dGTP, dCTP, dTTP and dUTP.

In some embodiments, a multivalent molecule comprises a core attached to multiple nucleotide arms, where each arm includes a nucleotide unit. The nucleotide unit comprises an aromatic base, a five carbon sugar (e.g., ribose or deoxyribose), and one or more phosphate groups (e.g., 1-10 phosphate groups). The plurality of multivalent molecules can comprise one type of multivalent molecule having one type of nucleotide unit selected from a group consisting of dATP, dGTP, dCTP, dTTP and dUTP. The plurality of multivalent molecules can comprise a mixture of any combination of two or more types of multivalent molecules, where individual multivalent molecules in the mixture comprise nucleotide units selected from a group consisting of dATP, dGTP, dCTP, dTTP and/or dUTP.

In some embodiments, the nucleotide unit comprises a chain of one, two or three phosphorus atoms where the chain is typically attached to the 5′ carbon of the sugar moiety via an ester or phosphoramide linkage. In some embodiments, at least one nucleotide unit is a nucleotide analog having a phosphorus chain in which the phosphorus atoms are linked together with intervening O, S, NH, methylene or ethylene. In some embodiments, the phosphorus atoms in the chain include substituted side groups including O, S or BH3. In some embodiments, the chain includes phosphate groups substituted with analogs including phosphoramidate, phosphorothioate, phosphordithioate, and O-methylphosphoroamidite groups.

In some embodiments, the multivalent molecule comprises a core attached to multiple nucleotide arms, and wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to 3′ sugar position where the sugar comprises a ribose or deoxyribose sugar moiety. In some embodiments, the chain terminating moiety is removable/cleavable from 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide unit, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh3)4) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K2CO3) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide unit comprises a chain terminating moiety (e.g., blocking moiety) at the sugar 2′ position, at the sugar 3′ position, or at the sugar 2′ and 3′ positions. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the nucleotide unit comprising a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′-butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein the nucleotide arms comprise a spacer, linker and nucleotide unit, and wherein the core, linker and/or nucleotide unit is labeled with detectable reporter moiety. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, at least one nucleotide arm of a multivalent molecule has a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, the core of a multivalent molecule comprises an avidin-like or streptavidin-like moiety and the core attachment moiety comprises biotin. In some embodiments, the core comprises a streptavidin-type or avidin-type moiety which includes an avidin protein, as well as any derivatives, analogs and other non-native forms of avidin that can bind to at least one biotin moiety. Other forms of avidin moieties include native and recombinant avidin and streptavidin as well as derivatized molecules, e.g. non-glycosylated avidin and truncated streptavidins. For example, an avidin moiety can include de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN, CAPTAVIDIN, NEUTRA VIDIN and NEUTRALITE AVIDIN.

In some embodiments, any of the methods for sequencing nucleic acid molecules described herein can include forming a binding complex, where the binding complex comprises (i) a polymerase, a nucleic acid template molecule duplexed with a primer, and a nucleotide, or the binding complex comprises (ii) a polymerase, a nucleic acid template molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.8, 0.9 or 1 second. The binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, and/or wherein the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water. In some embodiments, the present disclosure provides said method wherein the binding complex is deposited on, attached to, or hybridized to, a surface showing a contrast to noise ratio in the detecting operation of greater than 20. In some embodiments, the present disclosure provides said method wherein the contacting is performed under a condition that stabilizes the binding complex when the nucleotide or nucleotide unit is complementary to a next base of the template nucleic acid, and destabilizes the binding complex when the nucleotide or nucleotide unit is not complementary to the next base of the template nucleic acid.

### Methods for Sequencing Using Phosphate-Chain Labeled Nucleotides

In some embodiments, the methods herein can be used for autofocusing of optical systems that can be used for sequencing using immobilized sequencing polymerases which bind non-immobilized template molecules. The present disclosure provides methods for sequencing using immobilized sequencing polymerases which bind non-immobilized template molecules, wherein the sequencing reactions are conducted with phosphate-chain labeled nucleotides. In some embodiments, the sequencing methods comprise operation (a): providing a support having a plurality of sequencing polymerases immobilized thereon. In some embodiments, the sequencing polymerase comprises a processive DNA polymerase. In some embodiments, the sequencing polymerase comprises a wild type or mutant DNA polymerase, including for example a Phi29 DNA polymerase. In some embodiments, the support comprise a plurality of separate compartments and a sequencing polymerase is immobilized to the bottom of a compartment. In some embodiments, the separate compartments comprise a silica bottom through which light can penetrate. In some embodiments, the separate compartments comprise a silica bottom configured with a nanophotonic confinement structure comprising a hole in a metal cladding film (e.g., aluminum cladding film). In some embodiments, the hole in the metal cladding has a small aperture, for example, approximately 70 nm. In some embodiments, the height of the nanophotonic confinement structure is approximately 100 nm. In some embodiments, the nanophotonic confinement structure comprises a zero mode waveguide (ZMW). In some embodiments, the nanophotonic confinement structure contains a liquid.

In some embodiments, the sequencing method further comprises operation (b): contacting the plurality of immobilized sequencing polymerases with a plurality of single stranded circular nucleic acid template molecules and a plurality of oligonucleotide sequencing primers, under a condition suitable for individual immobilized sequencing polymerases to bind a single stranded circular template molecule, and suitable for individual sequencing primers to hybridize to individual single stranded circular template molecules, thereby generating a plurality of polymerase/template/primer complexes. In some embodiments, the individual sequencing primers hybridize to a universal sequencing primer binding site on the single stranded circular template molecule.

In some embodiments, the sequencing method further comprises operation (c): contacting the plurality of polymerase/template/primer complexes with a plurality of phosphate chain labeled nucleotides each comprising an aromatic base, a five carbon sugar (e.g., ribose or deoxyribose), and phosphate chain comprising 3-20 phosphate groups, where the terminal phosphate group is linked to a detectable reporter moiety (e.g., a fluorophore). The first, second and third phosphate groups can be referred to as alpha, beta and gamma phosphate groups. In some embodiments, a particular detectable reporter moiety which is attached to the terminal phosphate group corresponds to the nucleotide base (e.g., dATP, dGTP, dCTP, dTTP or dUTP) to permit detection and identification of the nucleo-base. In some embodiments, the plurality of polymerase/template/primer complexes are contacted with the plurality of phosphate chain labeled nucleotides under a condition suitable for polymerase-catalyzed nucleotide incorporation. In some embodiments, the sequencing polymerases are capable of binding a complementary phosphate chain labeled nucleotide and incorporating the complementary nucleotide opposite a nucleotide in a template molecule. In some embodiment, the polymerase-catalyzed nucleotide incorporation reaction cleaves between the alpha and beta phosphate groups thereby releasing a multi-phosphate chain linked to a fluorophore.

In some embodiments, the sequencing method further comprises operation (d): detecting the fluorescent signal emitted by the phosphate chain labeled nucleotide that is bound by the sequencing polymerase, and incorporated into the terminal end of the sequencing primer. In some embodiments, operation (d) further comprises identifying the phosphate chain labeled nucleotide that is bound by the sequencing polymerase, and incorporated into the terminal end of the sequencing primer.

In some embodiments, the sequencing method further comprises operation (d): repeating operations (c)-(d) at least once. In some embodiments, sequencing methods that employ phosphate chain labeled nucleotides can be conducted according to the methods described in U.S. Pat. Nos. 7,170,050; 7,302,146; and/or 7,405,281.

The headings provided herein are not limitations of the various aspects of the disclosure, which aspects can be understood by reference to the specification as a whole.

Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to conventional methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those well-known and commonly used in the art.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the terms “about,” “approximately,” and “substantially” refer to a value or composition that is within an acceptable error range for the particular value or composition as determined by one of ordinary skill in the art, which will depend in part on how the value or composition is measured or determined, e.g., the limitations of the measurement system. For example, “about,” “approximately,” or “substantially” can mean within one or more than one standard deviation per the practice in the art. Alternatively, “about” or “approximately” can mean a range of up to 10% (e.g., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about,” “approximately,” “substantially” should be assumed to be within an acceptable error range for that particular value or composition. Also, where ranges and/or subranges of values are provided, the ranges and/or subranges can include the endpoints of the ranges and/or subranges.

The term “polony” used herein refers to a nucleic acid library molecule can be clonally amplified in-solution or on-support to generate an amplicon that can serve as a template molecule for sequencing. In some embodiments, a linear library molecule can be circularized to generate a circularized library molecule, and the circularized library molecule can be clonally amplified in-solution or on-support to generate a concatemer. In some embodiments, the concatemer can serve as a nucleic acid template molecule which can be sequenced. The concatemer is sometimes referred to as a polony. In some embodiments, a polony includes nucleotide strands.

References herein to “one embodiment,” “an embodiment,” “an example embodiment,” “some embodiments,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein.

It is to be appreciated that the Detailed Description section, and not any other section, is intended to be used to interpret the claims. Other sections may set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries may be defined as long as the specified functions and relationships (or equivalents thereof) are appropriately performed. Also, alternative embodiments may perform functional blocks, steps, operations, methods, etc. using orderings different from those described herein.

### EXAMPLES

These examples are provided for illustrative purposes only and not to limit the scope of the claims provided herein. Non-limiting examples of sequencing methods and systems that can be used with the present disclosure can be found in PCT application Nos. PCT/US2023/081406 and PCT/US2020/034409, each of which is incorporated by reference herein in its entirety.

### Example 1—Prophetic Example of Using an Optical System

The purpose of this example is to demonstrate sequencing of a nucleic acid sequence using an optical system focused as described herein. Such an optical system and focusing method provides additional advantages and utility for nucleic acid sequencing applications due to reduced optical components, less moving parts, and higher throughput.

In this example, a flow cell is inserted into the optical system. The flow cell can be tilted out of the focal plane of the optical system and an image of the flow cell taken with an imaging detector of an autofocusing element of the optical system. The image is then processed using a computer processor operatively coupled to the detector, determining an amount of defocus in the flow cell using the distance of the in focus portion of the image from the center of the image. The optical system then de-tilts the flow cell, moves the flow cell by the amount of defocus to place the flow cell in the focal plane of the optical system.

A sample is delivered to a hydrophobic pad of a flow cell by a liquid handling system. The sample is drawn into an interior channel of the flow cell by a vacuum pump. Nucleic acid sequences present in the sample react with primers attached to walls of the interior channel of the flow cell. The nucleic acid sequences of the sample are then amplified and washed. After amplification and washing, a solution containing: (1) DAPI modified nucleotide conjugate complementary to A nucleotides; (2) FITC modified nucleotide conjugate complementary to G nucleotides; (3) TRITC modified nucleotide conjugate complementary to C nucleotides; and (4) a fourth nucleotide conjugate modified with both DAPI and TRITC that is complementary toward T nucleotides is introduced to the flow cell 4521 and allowed to react with the primed nucleic acid sequence. The sample in the flow cell 4521 is then illuminated by a 0.1 second pulse of UV-blue light via a first LED light source 4522 thus exciting the DAPI fluorophore. In synchronization with the UV-blue light pulse, the imaging sensors acquire a first image capturing emission of light given off by any DAPI modified nucleotide conjugate bound specifically to the sample. Only light emitted by DAPI fluorescence emission is collected by the imaging sensors because the UV-blue excitation light emitted by the first light source is negligible past 405 nm. This light is blocked by a tri-band bandpass filter (Edmund Scientific stock #87-236) with multi-band center wavelengths at 432 nm, 517 nm and 615 nm. For this filter, bandwidths are 36 @ 432 nm, 23 @ 517 nm and 61 @ 615 nm. Next, the sample is pulsed with 0.1 seconds of green light via a second LED light source 4523, capable of exciting the FITC fluorophore. In synchronization with the green light pulse, a second image is acquired capturing emission of light given off by FITC modified nucleotide conjugate bound specifically to the sample. The sample can then be pulsed with 0.1 seconds of red light via a third LED light source 4524 thus exciting the TRITC fluorophore. In synchronization with the red light pulse, a third image is acquired capturing emission of light given off by any TRITC modified nucleotide conjugate bound specifically to the sample. In this example, excitation filters are used for each LED light source to minimize fluorescence channel cross-talk, or bleed-through of the excitation light into the emission bandpasses (notches) of the tri-band bandpass filter.

In this example, the base calling process is as follows. The first image of the cycle is analyzed for regions of interest (ROI) showing strong fluorescence signal. ROI's showing strong fluorescence signal in the first image indicate nucleic acid amplicons with either A or T at the open position prior to exposure to the nucleotide conjugates for the following reason. Capture of the first image was synchronized with sample illumination by UV-blue light, thus exciting DAPI. Since the nucleotide conjugates complementary toward A were labeled with DAPI and nucleotide conjugates complementary toward T were labeled with both DAPI and TRITC, ROI's of the first image showing strong fluorescence indicate either an A or T. Next, the second image of the cycle is analyzed for ROI's strong fluorescence signal. Since nucleotide conjugates complementary toward G were labeled with FITC and since capture of the second image was synchronized with the green pulse capable of exciting FTIC, ROI's in the second image showing strong fluorescent signal indicate G. Next, the third image of the cycle is analyzed for ROI's strong fluorescence signal. These ROI's indicate nucleic acid amplicons with either C or T present at the open position prior to exposure to the nucleotide conjugates. This is because in synchronization with the capture of the third image, the sample is illuminated with red light, thus exciting TRITC. Nucleotide conjugates complementary to C are labeled with TRITC and nucleotide conjugates complementary toward T are labeled with both DAPI and TRITC. ROI's with strong fluorescence signal observed in both the first and third image indicate a T nucleotide at the open position prior to exposure to the nucleotide conjugates. Identification of ROI's containing T's then allows for identification ROI's containing of A and C. The sequencing and imaging cycle is repeated until the entire nucleic acid sequence has been identified.

### Example 2—Prophetic Example of Using a Super Resolution Enhanced Optical System

The purpose of this example is to demonstrate sequencing of a nucleic acid sequence using a super resolution enhanced optical system as described herein. Such a system provides additional advantages and utility for nucleic acid sequencing applications due to reduced optical components, less moving parts and higher throughput, while providing for super high-resolution readout.

In this example, a flow cell is inserted into the optical system. The flow cell can be tilted out of the focal plane of the optical system and an image of the flow cell taken with an imaging detector of an autofocusing element of the optical system. The image is then processed using a computer processor operatively coupled to the detector, determining an amount of defocus in the flow cell using the distance of the in focus portion of the image from the center of the image. The optical system then de-tilts the flow cell, moves the flow cell by the amount of defocus to place the flow cell in the focal plane of the optical system.

A sample is delivered to capillary flow cell. Sample sites comprising nucleic acid sequences present in the sample react with primers attached to walls of the interior channel of the capillary flow cell. The nucleic acid sequences of the sample are then amplified and washed. After amplification and washing, a solution containing (1) DAPI modified nucleotide conjugate complementary to A nucleotides; (2) FITC modified nucleotide conjugate complementary to G nucleotides; (3) TRITC modified nucleotide conjugate complementary to C nucleotides; and (4) a fourth nucleotide conjugate modified with both DAPI and TRITC that is complementary toward T nucleotides is introduced to the capillary flow cell and allowed to react with the primed nucleic acid sequence. The sample in the capillary flow cell is then illuminated by a 0.1 second pulse of UV-blue light via a first LED light source of the light source thus exciting the DAPI fluorophore. In synchronization with the UV-blue light pulse, the imaging sensors acquire a first image capturing emission of light given off by any DAPI modified nucleotide conjugate bound specifically to the sample. Only light emitted by DAPI fluorescence emission is collected by the imaging sensors because the UV-blue excitation light emitted by the first light source is negligible past 405 nm. This light is blocked by a tri-band band stop filter. Next, the sample is pulsed with 0.1 seconds of green light via a second LED light source of the light source, capable of exciting the FITC fluorophore. In synchronization with the green light pulse, a second image is acquired capturing emission of light given off by FITC modified nucleotide conjugate bound specifically to the sample. The sample is pulsed with 0.1 seconds of red light via a third LED light source of the light source thus exciting the TRITC fluorophore. In synchronization with the red light pulse, a third image is acquired capturing emission of light given off by any TRITC modified nucleotide conjugate bound specifically to the sample. In this example, excitation filters are used for each LED light source to minimize fluorescence channel cross-talk, or bleed-through of the excitation light that may not be stopped by the notches, or band stops of the tri-band band stop filter.

A wedge block can be included in each optical subsystem in order to image the entire inner surface of the capillary flow cell 5201. When the top-wedge piece is aligned with the bottom wedge piece the optical subsystems acquire images on the far side of the inner surface of the capillary flow cell. When the top-wedge piece is moved out of alignment to increase the optical pathlength the optical subsystems acquire images on the front interior surface of the capillary flow cell.

The optical system in this example is capable of super resolution imaging, wherein at least one sample site comprising clonally-amplified, sample nucleic acid molecules immobilized to a plurality of attached oligonucleotide molecules, wherein said plurality of immobilized clonally amplified sample nucleic acid molecules are present at distance less than λ/(2*NA), wherein λ is the center wavelength of an excitation energy source and NA is the numerical aperture of an imaging system. A stochastic photo-switching chemistry is then applied to said clonally amplified sample nucleic acid molecules at the same time to cause said plurality of clonally amplified sample nucleic acid molecules to fluoresce in on and off events in up to four different colors by stochastic photo-switching; and on and off events are detected in a color channel for each color in real-time as the on and off events are occurring for said plurality of clonally amplified sample nucleic acid molecules to determine an identify of a nucleotide of said clonally amplified sample nucleic acid molecule.

In this example, the base calling process is as follows. The first image of the cycle is analyzed for regions of interest (ROI) showing strong fluorescence signal. ROI's showing strong fluorescence signal in the first image indicate nucleic acid amplicons with either A or T at the open position prior to exposure to the nucleotide conjugates for the following reason. Capture of the first image was synchronized with sample illumination by UV-blue light, thus exciting DAPI. Since the nucleotide conjugates complementary toward A were labeled with DAPI and nucleotide conjugates complementary toward T were labeled with both DAPI and TRITC, ROI's of the first image showing strong fluorescence indicate either an A or T. Next, the second image of the cycle is analyzed for ROI's strong fluorescence signal. Since nucleotide conjugates complementary toward G were labeled with FITC and since capture of the second image was synchronized with the green pulse capable of exciting FTIC, ROI's in the second image showing strong fluorescent signal indicate G. Next, the third image of the cycle is analyzed for ROI's strong fluorescence signal. These ROI's indicate nucleic acid amplicons with either C or T present at the open position prior to exposure to the nucleotide conjugates. This is because in synchronization with the capture of the third image, the sample is illuminated with red light, thus exciting TRITC. Nucleotide conjugates complementary to C are labeled with TRITC and nucleotide conjugates complementary toward T are labeled with both DAPI and TRITC. ROI's with strong fluorescence signal observed in both the first and third image indicate a T nucleotide at the open position prior to exposure to the nucleotide conjugates. Identification of ROI's containing T's then allows for identification ROI's containing of A and C. The sequencing and imaging cycle is repeated until the entire nucleic acid sequence has been identified.

### Numbered Embodiments of the Disclosure

1. A method for focusing an optical system, comprising:
   (a) receiving an image of a substrate of said optical system, wherein a portion and less than all of said image is in focus, and wherein said portion of said image in focus is offset from a center of said image;
   (b) determining, using at least a distance from said portion of said image in focus and said center of said image, an amount of defocus in said image; and
   (c) adjusting a parameter of said optical system to adjust for said defocus.
2. The method of any of the preceding embodiments, wherein said image is an image of a flow cell, and wherein said substrate is a flow cell.
3. The method of any of the preceding embodiments, wherein said adjusting of (c) is an automated adjusting.
4. The method of any of the preceding embodiments, wherein said image is received from an autofocus element.
5. The method of any of the preceding embodiments, wherein said determining is done in at most about 600 milliseconds (ms).
6. The method of any of the preceding embodiments, wherein said determining is done within at most about 100 ms.
7. The method of any of the preceding embodiments, further comprising, prior to (a), imaging a substrate using a light source and a detector to generate said image.
8. The method of any of the preceding embodiments, wherein said determining is performed using said image and no additional images.
9. The method of any of the preceding embodiments, wherein said image comprises a length or width that is in a range from about 0.1 millimeters (mm) to about 5 centimeters (cm).
10. The method of any of the preceding embodiments, wherein said image comprises a length or width that is in a range from about 0.5 mm to about 9 mm.
11. The method of any of the preceding embodiments, wherein an error in said amount of defocus from a true amount of defocus is at most about 400 nanometers (nm).
12. The method of any of the preceding embodiments, wherein an error in said amount of defocus from a true amount of defocus is at most about 100 nanometers (nm).
13. The method of any of the preceding embodiments, wherein an error in said amount of defocus from a true amount of defocus is at most about 50 nanometers (nm).
14. The method of any of the preceding embodiments, wherein a center of said in focus region is determined using an image processing algorithm.
15. The method of any of the preceding embodiments, wherein said image processing algorithm comprises determining said center of said in focus region by separating said image into a predetermined number of regions and using a sum or average intensity of each region to identify the location of said in focus region.
16. The method of any of the preceding embodiments, wherein image intensity or spatial frequency information of said location of said in focus region is used to locate said center of said in focus region.
17. The method of any of the preceding embodiments, wherein information about a geometrical pattern in said image determines said image processing algorithm.
18. A method of focusing an optical system, comprising:
   (a) imaging, using a detector, a substrate tilted at a tilt angle, wherein an image of said substrate comprises an in focus portion and an out-of-focus portion;
   (b) determining, using a processor, a defocus of the optical system based at least in part on said tilt angle and a distance of said in focus portion from a center of said image;
   (c) adjusting said substrate to remove said tilt angle; and
   (d) adjusting said substrate by said defocus, thereby focusing said optical system.
19. The method of any of the preceding embodiments, wherein said determining of (b) further comprising using a vector of said in focus portion from said center of said image.
20. The method of any of the preceding embodiments, further comprising a motor coupled to said substrate configured to impart said tilt angle.
21. The method of any of the preceding embodiments, wherein said detector is a portion of an autofocusing element.
22. The method of any of the preceding embodiments, wherein said optical system further comprises an additional detector configured to image said substrate.
23. The method of any of the preceding embodiments, further comprising, prior to (a), tilting said substate to said tilt angle.
24. The method of any of the preceding embodiments, further comprising, subsequent to (d), de-tilting said substrate.
25. The method of any of the preceding embodiments, wherein said tilting is tilting of a plane orthogonal to an optical axis of said optical system.
26. The method of any of the preceding embodiments, wherein said tilt angle is from about 0.01 to about 89 degrees.
27. The method of any of the preceding embodiments, wherein said tilt angle is from about 0.05 to about 15 degrees.
28. The method of any of the preceding embodiments, wherein an angular resolution of said tilt angle is from about 0.001 degrees to about 0.2 degrees.
29. The method of any of the preceding embodiments, wherein an angular resolution of said tilt angle is from about 0.01 degrees to about 0.1 degrees.
30. The method of any of the preceding embodiments, wherein an angular resolution of said tilt angle is from about 0.01 degrees to about 0.08 degrees.
31. The method of any of the preceding embodiments, wherein said determining is performed using said image and no additional images.
32. The method of any of the preceding embodiments, wherein said substrate comprises a flow cell comprising:
   (a) one or more surfaces;
   (b) at least one hydrophilic polymer coating layer;
   (c) a plurality of oligonucleotide molecules attached to said at least one hydrophilic polymer coating layer; and
   (d) at least one discrete region of said one or more surfaces that comprises a plurality of clonally-amplified nucleic acid molecules immobilized to said plurality of attached oligonucleotide molecules, wherein said plurality of immobilized clonally amplified sample nucleic acid molecules are present at distance less than λ/(2*NA), wherein A is the center wavelength of an excitation energy source and NA is the numerical aperture of said optical system.
33. The method of any of the preceding embodiments, wherein said substrate comprises a beaded flow cell.
34. The method of any of the preceding embodiments, wherein said beaded flow cell comprises a surface comprising fluorescent beads chemically immobilized to said substrate.
35. The method of any of the preceding embodiments, wherein said fluorescent beads are randomly distributed on said surface.
36. The method of any of the preceding embodiments, wherein said fluorescent beads comprise at least about 4 types of beads configured to emit different colors in response to excitation from a laser.
37. The method of any of the preceding embodiments, wherein an error in said distance from a focal plane to a true distance from said focal plane is at most about 400 nanometers (nm).
38. The method of any of the preceding embodiments, wherein an error in said distance from said focal plane to a true distance from said focal plane is at most about 100 nanometers (nm).
39. The method of any of the preceding embodiments, wherein an error in said distance from said focal plane to a true distance from said focal plane is at most about 50 nanometers (nm).
40. The method of any of the preceding embodiments, wherein (d) occurs prior to said optical system imaging a nucleic acid molecule immobilized to said substrate in a first flow cycle.
41. The method of any of the preceding embodiments, further comprising repeating (a)-(d) to refocus said optical system for a second flow cycle.
42. A method of focusing an optical system, comprising:
   (a) imaging, using a detector tilted at a tilt angle, a substrate, wherein an image of said substrate comprises an in focus portion and an out-of-focus portion;
   (b) determining, using a processor, a defocus of the optical system based at least in part on said tilt angle and a distance of said in focus portion from a center of said image; and
   (c) adjusting said substrate by said defocus, thereby focusing said optical system.
43. The method of any of the preceding embodiments, further comprising adjusting said substrate by said defocus, thereby placing said substrate into focus.
44. The method of any of the preceding embodiments, further comprising, prior to (a), tilting said detector to said tilt angle.
45. The method of any of the preceding embodiments, further comprising, subsequent to (c), de-tilting said detector.
46. The method of any of the preceding embodiments, wherein said tilting is tilting of a plane orthogonal to an optical axis of said optical system.
47. The method of any of the preceding embodiments, wherein said tilt angle is from about 0.01 to about 89 degrees.
48. The method of any of the preceding embodiments, wherein said tilt angle is from about 0.05 to about 15 degrees.
49. The method of any of the preceding embodiments, wherein said determining is performed using said image and no additional images.
50. The method of any of the preceding embodiments, wherein an error in said amount of defocus from a true amount of defocus is at most about 400 nanometers (nm).
51. The method of any of the preceding embodiments, wherein an error in said amount of defocus from a true amount of defocus is at most about 100 nanometers (nm).
52. The method of any of the preceding embodiments, wherein an error in said amount of defocus from a true amount of defocus is at most about 50 nanometers (nm).
53. The method of any of the preceding embodiments, further comprising calibrating a pivot point of the optical system.
54. The method of any of the preceding embodiments, wherein said calibrating of said pivot point comprises de-tilting said substrate, said detector, or an autofocus sensor.
55. An optical system, comprising:
   (a) a substrate;
   (b) an autofocus module configured to take an image of said substrate, wherein said image comprises an in focus portion and an out of focus portion, and wherein said substrate or said autofocus module is tilted at a tilt angle; and
   (c) a processor configured to determine a defocus of said substrate to a focal plane of said optical system using at least a distance from said in focus portion to a center of said image and said tilt angle.
56. The optical system of any of the preceding embodiments, wherein said substrate is tilted at a tilt angle.
57. The optical system of any of the preceding embodiments, wherein said processor uses said tilt angle in said determining said defocus.
58. The optical system of any of the preceding embodiments, wherein said autofocus module is tilted at a tilt angle.
59. The optical system of any of the preceding embodiments, wherein said processor uses said tilt angle in said determining said defocus.
60. The optical system of any of the preceding embodiments, wherein said autofocus module comprises an illumination source and a detector.
61. The optical system of any of the preceding embodiments, wherein said illumination source is configured to illuminate at least a portion of said substrate, and said detector is configured to image said portion of said substrate.
62. The optical system of any of the preceding embodiments, wherein said tilt angle is from about 0.01 to about 89 degrees.
63. The optical system of any of the preceding embodiments, wherein said tilt angle is from about 0.05 to about 15 degrees.
64. The optical system of any of the preceding embodiments, wherein said determining is performed using said image and no additional images.
65. The optical system of any of the preceding embodiments, wherein an error in said amount of defocus from a true amount of defocus is at most about 400 nanometers (nm).
66. The optical system of any of the preceding embodiments, wherein an error in said amount of defocus from a true amount of defocus is at most about 100 nanometers (nm).
67. The optical system of any of the preceding embodiments, wherein an error in said amount of defocus from a true amount of defocus is at most about 50 nanometers (nm).
68. The optical system of any of the preceding embodiments, wherein said autofocus module comprises one or more of an autofocus illumination source, an autofocus sensor, an autofocus tube lens, a dichroic filter, or a beam splitter.
69. The optical system of any of the preceding embodiments, wherein said optical system comprises one or more image sensors.
70. The optical system of any of the preceding embodiments, wherein said one or more image sensors are used for both imaging said substrate and focusing said optical system.
71. The optical system of any of the preceding embodiments, wherein said image is acquired by said autofocus module, and wherein said autofocus module is only configured for autofocusing and not for imaging said substrate after autofocusing is completed.
72. A method for autofocus of an optical system, comprising:
   tilting a sample stage of the optical system by a tilt angle, wherein a sample is immobilized on the sample stage;
   obtaining, by an image sensor of the optical system, an image of the sample on the tilted sample stage;
   determining, by a processor, a z shift based on: the tilt angle; and a x-y plane shift from a center of the image, wherein the x-y plane shift is determined based on an in-focus region of the image; and
   moving the sample stage relative to a focal plane of an objective lens of the optical system by the determined z shift thereby bringing the sample in-focus.
73. A method for autofocus of an optical system, comprising:
   tilting an image sensor of the optical system by a tilt angle;
   obtaining, by the tilted image sensor of the optical system, an image of a sample, wherein the sample is immobilized on a sample stage;
   determining, by a processor, a z shift based on: the tilt angle; and a x-y plane shift from a center of the image, wherein the x-y plane shift is determined based on an in-focus region of the image; and
   moving the sample stage relative to a focal plane of an objective lens of the optical system by the determined z shift thereby bringing the sample in-focus.
74. A method for autofocus of an optical system, comprising:
   tilting a sample stage of the optical system by a tilt angle, wherein a sample is immobilized on the sample stage;
   obtaining, by an autofocus (AF) sensor of the optical system, an image of the sample on the tilted sample stage, wherein the AF sensor is different from an image sensor of the optical system;
   determining, by a processor, a z shift based on the tilt angle and a x-y plane shift from a center of the image, wherein the x-y plane shift is determined based on an in-focus region of the image; and
   moving the sample stage relative to a focal plane of an objective lens of the optical system by the determined z shift thereby bringing the sample in-focus.
75. A method for autofocus of an optical system, comprising:
   tilting an AF sensor of the optical system by a tilt angle, wherein the AF sensor is different from an image sensor of the optical system;
   obtaining, by the AF sensor of the optical system, an image of the sample, wherein the sample is immobilized on a sample stage;
   determining, by a processor, a z shift based on the tilt angle and a x-y plane shift from a center of the image, wherein the x-y plane shift is determined based on an in-focus region of the image; and
   moving the sample stage relative to a focal plane of an objective lens of the optical system by the determined z shift thereby bring the sample in-focus with the optical system.
76. The method of any of the preceding embodiments, wherein the method further comprising: calibrating a pivot point of the optical system.
77. The method of any of the preceding embodiments, wherein calibrating the pivot point of the optical system comprises:
   tilting the sample stage, the image sensor, or the AF sensor by the tilt angle or a second tilt angle;
   acquiring, by the AF sensor or the image sensor, a calibration image of the sample immobilized on the sample stage;
   determining, by the processor, a pivot point offset based on a region center of an in-focus region of the calibration image and an image center of the calibration image; and
   de-tilting the sample stage, the image sensor, or the AF sensor by the tilt angle or the second tilt angle.
78. The method of any of the preceding embodiments, wherein the method further comprises: de-tilting the tilted sample stage by the tilt angle.
79. The method of any of the preceding embodiments, wherein the method further comprises: de-tilting the tilted image sensor by the tilt angle.
80. The method of any of the preceding embodiments, wherein the method further comprises: de-tilting the tilted AF sensor by the tilt angle.
81. The method of any of the preceding embodiments, wherein tilting the sample stage of the optical system by the tilt angle is about a x or y axis.
82. The method of any of the preceding embodiments, wherein tilting the sample stage of the optical system by the tilt angle is within a x-z plane or y-z plane.
83. The method of any of the preceding embodiments, wherein tilting the AF sensor or image sensor of the optical system by the tilt angle is about x or y axis.
84. The method of any of the preceding embodiments, wherein tilting the AF sensor or image sensor of the optical system by the tilt angle is within a x-z plane or y-z plane.
85. The method of any of the preceding embodiments, wherein the tilt angle is in a range from 0.01 degrees to 89 degrees.
86. The method of any of the preceding embodiments, wherein the tilt angle is in a range from 0.05 degrees to 15 degrees.
87. The method of any of the preceding embodiments, wherein the tilt angle is clockwise about the x or y axis.
88. The method of any of the preceding embodiments, wherein the tilt angle is counter-clockwise about the x or y axis.
89. The method of any of the preceding embodiments, wherein the image of the sample obtained by the AF sensor or image sensor comprises a single image.
90. The method of any of the preceding embodiments, wherein the AF sensor is only used for acquiring signals for autofocusing the optical system.
91. The method of any of the preceding embodiments, wherein the image sensor is used for autofocusing the optical system and for imaging using the optical system after autofocusing.
92. The method of any of the preceding embodiments, wherein the optical system lacks an AF illumination source that is only used for autofocusing but not for imaging.
93. The method of any of the preceding embodiments, wherein the method for autofocus of the optical system is completed in 100 to 990 milliseconds.
94. The method of any of the preceding embodiments, wherein the method for autofocus of the optical system is completed in less than 600 milliseconds.
95. The method of any of the preceding embodiments, wherein the image comprises a field of view (FOV) that is identical to a size of the image sensor or the AF sensor along the x or y axis.
96. The method of any of the preceding embodiments, wherein the image comprises a length or width that is in a range from 0.1 mm to 5 cm.
97. The method of any of the preceding embodiments, wherein the image comprises a length or width that is in a range from 0.5 mm to 9 mm.
98. The method of any of the preceding embodiments, wherein the image comprises a field of view (FOV) that is identical to a size of the image sensor or the AF sensor along the x axis when the tilt angle is about the x axis and along the y axis when the tilt angle is about the y axis.
99. The method of any of the preceding embodiments, wherein the AF illumination source comprises a laser.
100. The method of any of the preceding embodiments, wherein the image comprises fluorescent signal from the sample.
101. The method of any of the preceding embodiments, wherein the sample comprises a flow cell comprising:
   one or more surfaces and one or more substrates;
   at least one hydrophilic polymer coating layer;
   a plurality of oligonucleotide molecules attached to at least one hydrophilic polymer coating layer; and
   at least one discrete region of the one or more surfaces that comprises a plurality of clonally-amplified, sample nucleic acid molecules immobilized to the plurality of attached oligonucleotide molecules, wherein the plurality of immobilized clonally amplified sample nucleic acid molecules are present at distance less than λ(2*NA), wherein λ is the center wavelength of an excitation energy source and NA is the numerical aperture of the optical system.
102. The method of any of the preceding embodiments, wherein the sample comprises a beaded flow cell.
103. The method of any of the preceding embodiments, wherein the beaded flow cell comprises a surface coated with fluorescent beads that are chemically immobilized to the surface.
104. The method of any of the preceding embodiments, wherein the fluorescent beads are randomly distributed on the surface.
105. The method of any of the preceding embodiments, wherein the fluorescent beads comprise one, two, three, four, five or six different types of beads that emit different colors in response to a laser excitement.
106. The method of any of the preceding embodiments, wherein the fluorescent beads emit fluorescent light of one or more wavelengths in response to laser excitement.
107. The method of any of the preceding embodiments, wherein the sample comprises a test target.
108. The method of any of the preceding embodiments, wherein the test target comprises a coating of predetermined geometric shapes or patterns that are spatially repeated.
109. The method of any of the preceding embodiments, wherein the predetermined geometric patterns or shapes are repeated in one or two dimensions.
110. The method of any of the preceding embodiments, wherein the test target lacks a flow cell and a liquid.
111. The method of any of the preceding embodiments, wherein the test target comprises one or more substrates with a predetermined refractive index.
112. The method of any of the preceding embodiments, wherein the test target comprises a top substrate having a predetermined refractive index.
113. The system of any one of the preceding embodiments, wherein the test target comprises a bottom substrate.
114. The method of any of the preceding embodiments, wherein at least a portion of the first or second substrates comprises the coating with the predetermined geometric patterns or shapes.
115. The method of any of the preceding embodiments, wherein the thickness of the first substrate is configured to simulate the presence of a first hypothetical flow cell.
116. The method of any of the preceding embodiments, wherein the thickness of the top substrate is configured to permit imaging of the bottom surface of the first channel of the hypothetical first flow cell.
117. The method of any of the preceding embodiments, wherein the coating of the predetermined geometric shapes or patterns comprises optically opaque portions and transparent portions.
118. The method of any of the preceding embodiments, wherein the optical system comprises 1, 2, 3, 4, 5, or 6 detection channels.
119. The method of any of the preceding embodiments, wherein the optical system is configured to acquire flow cell images with a FOV of greater than 1.0 mm² after autofocusing of the optical system.
120. The method of any of the preceding embodiments, wherein the optical system comprises: the objective lens;
   the image sensor; and
   a numerical aperture (NA) of less than 0.6; and
   the processor configured to process the flow cell images to correct for optical aberration and generate an optical resolution that is about identical in the flow cell images.
121. The method of any of the preceding embodiments, wherein the optical system further comprises one or more illumination sources, wherein the one or more illumination sources lack an AF laser configured only for autofocusing of the optical system.
122. The method of any of the preceding embodiments, wherein the method for autofocus of the optical system is configured for focusing the optical system for imaging the sample nucleic acid molecules immobilized to the flow cell in a first flow cycle and a second flow cycle in the sequencing run.
123. The method of any of the preceding embodiments, wherein the method for autofocus of the optical system is configured for focusing the optical system for imaging the sample nucleic acid molecules immobilized to a first surface in a first flow cycle in a sequencing run, and for refocusing the optical system for imaging the sample nucleic acid molecules immobilized to the first surface or to a second surface in a second flow cycle in the sequencing run.
124. The method of any of the preceding embodiments, wherein the method for autofocus of the optical system is configured for focusing the optical system for imaging the sample nucleic acid molecules immobilized to a first surface in a first flow cycle in a sequencing run, and for refocusing the optical system for imaging the sample nucleic acid molecules immobilized to a second surface in the first flow cycle or a second flow cycle in the sequencing run.
125. The method of any of the preceding embodiments, wherein the method for autofocus of the optical system is configured for focusing at least along a z axis.
126. The method of any of the preceding embodiments, wherein tilting the sample stage of the optical system by the tilt angle comprises:

tilting the sample stage of the optical system by the tilt angle simultaneously as moving the sample stage within the x-y plane.
127. The method of any of the preceding embodiments, wherein moving the sample stage within the x-y plane comprises moving the sample stage to a predetermined spatial location.
128. The method of any of the preceding embodiments, wherein de-tilting the tilted sample stage by the tilt angle comprises:

de-tilting the tilted sample stage by the tilt angle simultaneously as moving the sample stage relative to the focal plane of the objective lens by the determined z shift.
129. The method of any of the preceding embodiments, wherein de-tilting the tilted image sensor by the tilt angle comprises:

de-tilting the tilted image sensor by the tilt angle simultaneously as moving the sample stage by the determined z shift.
130. The method of any of the preceding embodiments, wherein de-tilting the tilted AF sensor by the tilt angle comprises:

de-tilting the tilted AF sensor by the tilt angle simultaneously as moving the sample stage by the determined z shift.
131. The method of any of the preceding embodiments, wherein an error in autofocusing the optical system is in the range from −400 nm to +400 nm.
132. The method of any of the preceding embodiments, wherein an error in autofocusing the optical system is in the range from −100 nm to +100 nm.
133. The method of any of the preceding embodiments, wherein an error in autofocusing the optical system is in the range from −50 nm to +50 nm.
134. The method of any of the preceding embodiments, wherein the sample stage is a motorized stage that automatically tilts by a predetermined angle provided by a user.
135. The method of any of the preceding embodiments, wherein the image sensor or the AF sensor is immobilized on a motorized stage that automatically tilts by a predetermined angle provided by a user.
136. The method of any of the preceding embodiments, wherein the objective lens is immobilized on a z-stage that is movable along the z-axis.
137. The method of any of the preceding embodiments, wherein moving the sample stage relative to a focal plane of an objective lens of the optical system by the determined z shift comprises moving the objective lens thereby moving the focal plane of the objective lens by the determined z shift.
138. The method of any of the preceding embodiments, wherein tilting the sample stage of the optical system by the tilt angle comprises:

receiving, by a motor coupled to the sample stage, the tilt angle; and
139. tilting, by the motor, the sample stage by the tilt angle. A method for autofocus of an optical system, comprising:
   acquiring, by the optical system, one or more flow cell images of a first tile or subtile of the sample in a flow cycle of a sequencing run;
   moving the sample stage to position a second tile or subtile next to the first tile or subtile sample relative to the optical system;
   repeating the method for autofocusing of the optical system in any one of the preceding embodiments; and
   acquiring, by the optical system, one or more flow cell images of the second tile or subtile of the sample in the flow cycle of the sequencing run.
140. An optical system comprising:
   an objective lens;
   a sample stage;
   an image sensor, wherein at least one of the sample stage and the image sensor is tiltable by a tilt angle;
   a numerical aperture (NA) of less than 0.6; and
   a processor configured to determining a z shift based on: the tilt angle; and a x-y plane shift from a center of an image acquired by the at least one image sensor,
   wherein the x-y plane shift is determined based on an in-focus region of the image, and wherein the z shift of the sample stage is configured to focus the sample stage to a focal plane of the objective lens.
141. An optical system comprising:
   an objective lens;
   a sample stage;
   an image sensor,
   an AF sensor, wherein at least one of the sample stage and the AF sensor is tiltable by a tilt angle;
   a numerical aperture (NA) of less than 0.6; and
   a processor configured to determining a z shift based on: the tilt angle; and a x-y plane shift from a center of an image acquired by the AF sensor,
   wherein the x-y plane shift is determined based on an in-focus region of the image, and wherein the z shift of the sample stage is configured to focus the sample stage to a focal plane of the objective lens.
142. The optical system of any of the preceding embodiments, wherein the sample stage, the AF sensor, the image sensor, or their combinations are tiltable about a x or y axis.
143. The optical system of any of the preceding embodiments, wherein the sample stage, the AF sensor, the image sensor, or their combinations are tiltable within a x-z plane or y-z plane.
144. The optical system of any of the preceding embodiments, wherein the sample stage, the AF sensor, the image sensor, or their combinations are tiltable by a motor connected thereto.
145. The optical system of any of the preceding embodiments, wherein the tilt angle is in a range from 0.01 degrees to 89 degrees.
146. The optical system of any of the preceding embodiments, wherein the tilt angle is in a range from 0.05 degrees to 15 degrees.
147. The optical system of any of the preceding embodiments, wherein the tilt angle is clockwise about the x or y axis.
148. The optical system of any of the preceding embodiments, wherein the tilt angle is counter-clockwise about the x or y axis.
149. The optical system of any of the preceding embodiments, wherein the image of the sample obtained by the AF sensor or image sensor comprises a single image.
150. The optical system of any of the preceding embodiments, wherein the AF sensor is only used for acquiring signals for autofocusing the optical system.
151. The optical system of any of the preceding embodiments, wherein the image sensor is used for autofocusing the optical system and for imaging using the optical system after autofocusing.
152. The optical system of any of the preceding embodiments, wherein the optical system lacks an AF illumination source that is only used for autofocusing but not for imaging.
153. The optical system of any of the preceding embodiments, wherein the AF illumination source comprises a laser.
154. The optical system of any of the preceding embodiments, wherein the optical system is configured for completing autofocus of the optical system in 100 to 990 milliseconds.
155. The optical system of any of the preceding embodiments, wherein the optical system is configured for completing autofocus of the optical system in less than 600 milliseconds.
156. The optical system of any of the preceding embodiments, wherein the image comprises a field of view (FOV) that is identical to a size of the image sensor or the AF sensor along the x or y axis.
157. The optical system of any of the preceding embodiments, wherein the image comprises a length or width that is in a range from 0.1 mm to 5 cm.
158. The optical system of any of the preceding embodiments, wherein the image comprises a length or width that is in a range from 0.5 mm to 9 mm.
159. The optical system of any of the preceding embodiments, wherein the image comprises a field of view (FOV) that is identical to a size of the image sensor or the AF sensor along the x axis when the tilt angle is about the x axis and along the y axis when the tilt angle is about the y axis.
160. The optical system of any of the preceding embodiments, wherein the image comprises fluorescent signal from the sample.
161. The optical system of any of the preceding embodiments, wherein the sample stage comprises a sample immobilized thereon during imaging of the sample using the optical system.
162. The optical system of any of the preceding embodiments, wherein the sample comprises a flow cell comprising:
   one or more surfaces and one or more substrates;
   at least one hydrophilic polymer coating layer;
   a plurality of oligonucleotide molecules attached to at least one hydrophilic polymer coating layer; and
   at least one discrete region of the one or more surfaces that comprises a plurality of clonally-amplified, sample nucleic acid molecules immobilized to the plurality of attached oligonucleotide molecules, wherein the plurality of immobilized clonally amplified sample nucleic acid molecules are present at distance less than λ/(2*NA), wherein λ is the center wavelength of an excitation energy source and NA is the numerical aperture of an imaging system.
163. The optical system of any of the preceding embodiments, wherein the sample comprises a beaded flow cell.
164. The optical system of any of the preceding embodiments, wherein the beaded flow cell comprises a surface coated with fluorescent beads that are chemically immobilized to the surface.
165. The optical system of any of the preceding embodiments, wherein the fluorescent beads are randomly distributed on the surface.
166. The optical system of any of the preceding embodiments, wherein the fluorescent beads comprise one, two, three, four, five or six different types of beads that emit different colors in response to a laser excitement.
167. The optical system of any of the preceding embodiments, wherein the fluorescent beads emit fluorescent light of one or more wavelengths in response to laser excitement.
168. The optical system of any of the preceding embodiments, wherein the sample comprises a test target.
169. The optical system of any of the preceding embodiments, wherein the test target comprises a coating of predetermined geometric shapes or patterns that are spatially repeated.
170. The optical system of any of the preceding embodiments, wherein the predetermined geometric patterns or shapes are repeated in one or two dimensions.
171. The optical system of any of the preceding embodiments, wherein the test target lacks a flow cell and a liquid.
172. The optical system of any of the preceding embodiments, wherein the test target comprises one or more substrates with a predetermined refractive index.
173. The optical system of any of the preceding embodiments, wherein the test target comprises a top substrate having a predetermined refractive index.
174. The optical system of any of the preceding embodiments, wherein the test target comprises a bottom substrate.
175. The optical system of any of the preceding embodiments, wherein at least a portion of the first or second substrates comprises the coating with the predetermined geometric patterns or shapes.
176. The optical system of any of the preceding embodiments, wherein the thickness of the first substrate is configured to simulate presence of a first hypothetical flow cell.
177. The optical system of any of the preceding embodiments, wherein the thickness of the top substrate is configured to permit imaging of the bottom surface of the first channel of the hypothetical first flow cell.
178. The optical system of any of the preceding embodiments, wherein the coating the predetermined geometric shapes or patterns comprises optically opaque portions and transparent portions.
179. The optical system of any of the preceding embodiments, wherein the optical system comprises 1, 2, 3, 4, 5, or 6 detection channels.
180. The optical system of any of the preceding embodiments, wherein the optical system is configured to acquire flow cell images with a FOV of greater than 1.0 mm² after autofocusing of the optical system.
181. The optical system of any of the preceding embodiments, wherein the optical system is configured for focusing the optical system before imaging the sample nucleic acid molecules immobilized to the flow cell in a first flow cycle and a second flow cycle in the sequencing run.
182. The optical system of any of the preceding embodiments, wherein the optical system is configured for focusing the optical system before imaging the sample nucleic acid molecules immobilized to a first surface in a first flow cycle in a sequencing run, and for refocusing the optical system before imaging the sample nucleic acid molecules immobilized to the first surface or to a second surface in a second flow cycle in the sequencing run.
183. The optical system of any of the preceding embodiments, wherein the optical system is configured for focusing the optical system for imaging the sample nucleic acid molecules immobilized to a first surface in a first flow cycle in a sequencing run, and for refocusing the optical system for imaging the sample nucleic acid molecules immobilized to a second surface in the first flow cycle or a second flow cycle in the sequencing run.
184. The optical system of any of the preceding embodiments, wherein the optical system is configured for focusing the optical system at least along a z axis.
185. The optical system of any of the preceding embodiments, wherein an error in autofocusing the optical system is in the range from −400 nm to +400 nm.
186. The optical system of any of the preceding embodiments, wherein an error in autofocusing the optical system is in the range from −100 nm to +100 nm.
187. The optical system of any of the preceding embodiments, wherein an error in autofocusing the optical system is in the range from −50 nm to +50 nm.
188. The optical system of any of the preceding embodiments, wherein the sample stage is a motorized stage that automatically tilts by a predetermined angle provided by a user.
189. The optical system of any of the preceding embodiments, wherein the image sensor or the AF sensor is immobilized on a motorized stage that automatically tilts by a predetermined angle.
190. The optical system of any of the preceding embodiments, wherein the objective lens is immobilized on a z-stage that is movable along the z-axis.
191. The optical system of any of the preceding embodiments, wherein the optical system further comprises one or more illumination sources.

While preferred embodiments of the present invention have been shown and described herein, it will be obvious to those skilled in the art that such embodiments are provided by way of example only. The breadth and scope of this disclosure should not be limited by any of the above-described exemplary embodiments. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for focusing an optical system, comprising:
(a) receiving an image of a substrate of said optical system, wherein a portion and less than all of said image is in focus, and wherein said portion of said image in focus is offset from a center of said image;
(b) determining, using at least a distance from said portion of said image in focus and said center of said image, an amount of defocus in said image; and
(c) adjusting a parameter of said optical system to adjust for said defocus.

2.-3. (canceled)

4. The method of claim 1, wherein said image is received from an autofocus element.

5. The method of claim 1, wherein said determining is done in at most about 600 milliseconds (ms).

6. (canceled)

7. The method of claim 1, further comprising, prior to (a), imaging a substrate using a light source and a detector to generate said image.

8.-13. (canceled)

14. The method of claim 1, wherein a center of said portion that is in focus is determined using an image processing algorithm.

15. The method of claim 14, wherein said image processing algorithm comprises determining said center of said portion that is in focus by separating said image into a predetermined number of portions and using a sum or average intensity of each portion to identify a location of said portion that is in focus.

16. The method of claim 15, wherein image intensity or spatial frequency information of said location of said portion that is in focus is used to locate said center of said portion that is in focus.

17. The method of claim 14, wherein information about a geometrical pattern in said image determines said image processing algorithm.

18. A method of focusing an optical system, comprising:
(a) imaging, using a detector, a substrate tilted at a tilt angle, wherein an image of said substrate comprises an in focus portion and an out-of-focus portion;
(b) determining, using a processor, a defocus of the optical system based at least in part on said tilt angle and a distance of said in focus portion from a center of said image;
(c) adjusting said substrate to remove said tilt angle; and
(d) adjusting said substrate by said defocus, thereby focusing said optical system.

19. The method of claim 18, wherein said determining of (b) further comprises using a vector of said in focus portion from said center of said image.

20. The method of claim 18, the optical system further comprising a motor coupled to said substrate configured to impart said tilt angle.

21. The method of claim 18, wherein said detector is a portion of an autofocusing element of the optical system.

22.-31. (canceled)

32. The method of claim 18, wherein said substrate comprises a flow cell comprising:
(a) one or more surfaces;
(b) at least one hydrophilic polymer coating layer;
(c) a plurality of oligonucleotide molecules attached to said at least one hydrophilic polymer coating layer; and
(d) at least one discrete region of said one or more surfaces that comprises a plurality of clonally-amplified nucleic acid molecules immobilized to said plurality of attached oligonucleotide molecules, wherein said plurality of immobilized clonally amplified sample nucleic acid molecules are present at a distance less than λ/(2*NA), wherein λ is the center wavelength of an excitation energy source and NA is the numerical aperture of said optical system.

33. The method of claim 32, wherein said substrate comprises a beaded flow cell.

34. The method of claim 33, wherein said beaded flow cell comprises a surface comprising fluorescent beads chemically immobilized to said substrate.

35. The method of claim 34, wherein said fluorescent beads are randomly distributed on said surface.

36. The method of claim 34, wherein said fluorescent beads comprise at least about 4 types of beads configured to emit different colors in response to excitation from a laser.

37.-41. (canceled)

42. A method of focusing an optical system, comprising:
(a) imaging, using a detector tilted at a tilt angle, a substrate, wherein an image of said substrate comprises an in focus portion and an out-of-focus portion;
(b) determining, using a processor, a defocus of the optical system based at least in part on said tilt angle and a distance of said in focus portion from a center of said image; and
(c) adjusting said substrate by said defocus, thereby focusing said optical system.

43. The method of claim 42, further comprising adjusting said substrate by said defocus, thereby placing said substrate into focus.

44. The method of claim 42, further comprising, prior to (a), tilting said detector to said tilt angle in a plane orthogonal to an optical axis of said optical system.

45. The method of claim 42, further comprising, subsequent to (c), de-tilting said detector in a plane orthogonal to an optical axis of said optical system.

46.-52. (canceled)

53. The method of claim 42, further comprising calibrating a pivot point of the optical system.

54. The method of claim 53, wherein said calibrating of said pivot point comprises de-tilting said substrate, said detector, or an autofocus sensor.
